SEQUENCE LISTING (29 sequences)

SEQ ID NO: 1 - protein, 39 aa, Homo sapiens
GTINVHDVETQFNQYKTEAASPYNLTISDVSVSHHHHHH

SEQ ID NO: 2 - protein, 51 aa, Homo sapiens
GTINVHDVETQFNQYKTEAASPYNLTISDVSVSDVPFPFSAQSGAHHHHHH

SEQ ID NO: 3 - protein, 54 aa, Homo sapiens
VQLTLAFREGTINVHDVETQFNQYKTEAASPYNLTISDVSVSDVPFPFHHHHHH

SEQ ID NO: 4 - protein, 31 aa, Homo sapiens
HHHHHHGFLGLSNIKFRPGSVVVQLTLAFRE

SEQ ID NO: 5 - protein, 46 aa, Homo sapiens
PDTRPAPGSTAPPAHGVTSAPDTRPAPGSTAPPAHGVTSAHHHHHH

SEQ ID NO: 6 - protein, 33 aa, Homo sapiens
GTINVHDVETQFNQYKTEAASPYNLTISDVSVS

SEQ ID NO: 7 - protein, 45 aa, Homo sapiens
GTINVHDVETQFNQYKTEAASPYNLTISDVSVSDVPFPFSAQSGA

SEQ ID NO: 8 - protein, 25 aa, Homo sapiens
GFLGLSNIKFRPGSVVVQLTLAFRE

SEQ ID NO: 9 - protein, 40 aa, Homo sapiens
PDTRPAPGSTAPPAHGVTSAPDTRPAPGSTAPPAHGVTSA

SEQ ID NO: 10 - protein, 1255 aa, Homo sapiens
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSSVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAAASANL

SEQ ID NO: 11 - protein, 302 aa, Homo sapiens
AAAKEGKKSRDRERPPSVPALREQPPETEPQPAWKMPRSCCSRSGALLLALLLQASMEVRGWCLESSQCQDLTTESNLLECIRACKPDLSAETPMFPGNGDEQPLTENPRKYVMGHFRWDRFGRRNSSSSGSSGAGQKREDVSAGEDCGPLPEGGPEPRSDGAKPGPREGKRSYSMEHFRWGKPVGKKRRPVKVYPNGAEDESAEAFPLEFKRELTGQRLREGDGPDGPADDGAGAQADLEHSLLVAAEKKDEGPYRMEHFRWGSPPKDKRYGGFMTSEKSQTPLVTLFKNAIIKNAYKKGE

SEQ ID NO: 12 - protein, 31 aa, Homo sapiens
HHHHHHSSSSGSSSSGSSSSGGRGDSGRGDS

SEQ ID NO: 13 - protein, 45 aa, Homo sapiens
GTINVHDVETQFNQYKTEAASRYNLTISDVSVSDVPFPFSAQSGA

SEQ ID NO: 14 - protein, 146 aa, Homo sapiens
GTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAAASANL

SEQ ID NO: 15 - protein, 171 aa, Homo sapiens
GFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAAASANL

SEQ ID NO: 16 - protein, 275 aa, Homo sapiens
ATTTPASKSTPFSIPSHHSDTPTTLASHSTKTDASSTHHSTVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAAASANL

SEQ ID NO: 17 - protein, 180 aa, Homo sapiens
GSGHASSTPGGEKETSATQRSSVPSSTEKNAFNSSLEDPSTDYYQELQRDISEMFLQIYKQGGFLGLSNIKFRPGSVVVQLTLAFREGTINVHDMETQFNQYKTEAASRYNLTISDVSVSDVPFPFSAQSGAGVPGWGIALLVLVCVLVALAIVYLIALAVCQSSLSYTNPAVAATSANL

SEQ ID NO: 18 - protein, 383 aa, Homo sapiens
LDPRVRTSAPDTRPAPGSTAPQAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSSVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAAASANL

SEQ ID NO: 19 - protein, 19 aa, Homo sapiens
MTPGTQSPFFLLLLLTVLT

SEQ ID NO: 20 - protein, 23 aa, Homo sapiens
MTPGTQSPFFLLLLLTVLTVVTA

SEQ ID NO: 21 - protein, 23 aa, Homo sapiens
MTPGTQSPFFLLLLLTVLTVVTG

SEQ ID NO: 22 - protein, 50 aa, Homo sapiens
TINVHDVETQFNQYKTEAASPYNLTISDVSVSDVPFPFSAQSGAHHHHHH

SEQ ID NO: 23 - protein, 63 aa, Homo sapiens
SVVVQLTLAFREGTINVHDVETQFNQYKTEAASPYNLTISDVSVSDVPFPFSAQSGAHHHHHH

SEQ ID NO: 24 - protein, 19 aa, Homo sapiens
HHHHHHSVVVQLTLAFREG

SEQ ID NO: 25 - protein, 44 aa, Homo sapiens
TINVHDVETQFNQYKTEAASRYNLTISDVSVSDVPFPFSAQSGA

SEQ ID NO: 26 - protein, 44 aa, Homo sapiens
TINVHDVETQFNQYKTEAASPYNLTISDVSVSDVPFPFSAQSGA

SEQ ID NO: 27 - protein, 13 aa, Homo sapiens
SVVVQLTLAFREG

SEQ ID NO: 28 - protein, 57 aa, Homo sapiens
SVVVQLTLAFREGTINVHDVETQFNQYKTEAASPYNLTISDVSVSDVPFPFSAQSGA

SEQ ID NO: 29 - protein, 51 aa, Homo sapiens
GTINVHDVETQFNQYKTEAASRYNLTISDVSVSDVPFPFSAQSGAHHHHHH

(19) United States
(12) Patent Application Publication
(10) Pub. No.: US 2008/0064680 A1
(43) Pub. Date: Mar. 13, 2008

(54) Methods for Diagnosis and Treatment of Cancer

(76) Inventor: Cynthia C. Bamdad, Boston, MA (US)

Correspondence Address: Jhk Law, P.o. box 1078, La Canada, 91012-1078, omitted

(21) Appl. No.: 10/564,981

(22) PCT Filed: Sep. 14, 2005
(86) PCT No.: PCT/US05/32821

§ 371 Date: Jan. 13, 2006

Related U.S. Application Data
(60) Provisional application No. 60/610,038, filed on Sep. 14, 2004.

(51) Int. Cl. A61K 31/517; A61K 31/341; A61K 31/437; A61P 35/00; G01N 33/53; A61K 31/55; A61K 31/496

(52) U.S. Cl. 514/212.03; 435/7.1; 514/252.17; 514/254.1; 514/266.3; 514/292; 514/471

(57) ABSTRACT

The present application discloses methods of detecting and treating cancer which express MUC1 aberrantly.

### CROSS-REFERENCE TO RELATED APPLICATIONS

The present application is a continuation-in-part application of U.S. patent application Ser. No. 09/996,069, filed Nov. 27, 2001, which claims the benefit of priority to U.S. Provisional Patent Application Ser. Nos. 60/253,361, filed Nov. 27, 2000; 60/256,027, filed Dec. 15, 2000; 60/258,157, filed Dec. 22, 2000; 60/259,615, filed Jan. 3, 2001; 60/260,186, Jan. 5, 2001; 60/266,169, filed Feb. 2, 2001; 60/289,444, filed May 7, 2001; 60/266,929, filed Feb. 6, 2001; 60/278,093, filed Mar. 23, 2001; 60/294,887, filed May 31, 2001; and 60/298,272, filed Jun. 14, 2001. The present application is also a continuation-in-part application of U.S. patent application Ser. No. 10/237,150, filed Sep. 5, 2002, which claims the benefit of priority to U.S. Provisional Patent Application Ser. Nos. 60/317,302, filed Sep. 5, 2001 and 60/376,732, filed May 1, 2002. The present application is also a continuation-in-part application of U.S. patent application Ser. No. 10/236,863, filed Sep. 5, 2002, which claims the benefit of priority to U.S. Provisional Patent Application No. 60/317,302, filed Sep. 5, 2001. The present application is also a continuation-in-part application of PCT/US2004/027954, filed Aug. 26, 2004, which claims the benefit of priority to U.S. Provisional Patent Application No. 60/498,260, filed Aug. 2, 2003. The present application also claims the benefit of priority to U.S. Provisional Patent Application No. 60/610,038, filed Sep. 14, 2004.

### BACKGROUND OF THE INVENTION

1. Field of the Invention

This invention generally relates to methods and compositions for the diagnosis and treatment of cancers that are characterized by the presence of the MUC1 receptor and, in particular, to cancers that are characterized by the aberrant expression of the MUC1 receptor. The invention also relates to methods for diagnosing and tracking a patient's response to therapy for MUC1-positive cancers.

2. Description of the Related Art

As our knowledge of cancer grows, it has become increasingly clear that cancer is not a single disease, but rather a collection of diseases that share some common characteristics. Indeed, both the treatment and the characterization of cancers are changing rapidly as causative factors are identified at the molecular level and molecular “signatures” of sub-types of cancer are discovered. The treatment of breast cancers is being increasingly designed to target specific molecular signatures that are present in that particular cancer and in that particular patient. Excised breast tumors are often tested to determine whether they present, or present elevated levels, of estrogen receptor, progesterone-receptor, or more recently the Her2/neu receptor. The characterization of tumors at the molecular level guides the physician in the choice of possible treatments for a particular patient. Therapies that are molecularly tuned to a particular patient have had a measurable impact of cancer recurrence and survival. For example, patients with estrogen-receptor positive (ER+) and/or progesterone-receptor positive (PR+) cancers are typically treated with Tamoxifen for a period of up to 5 years. Tamoxifen, an estrogen analog, works by binding to and blocking the estrogen receptor's natural estrogen docking site. The recurrence rate of the cancer is dependent on several factors, but in general, patients with cancers that are both ER+ and PR+ fare better (efficacy ˜70%) than those that are either ER+ or PR+ (efficacy ˜30%), or ER−/PR− (efficacy ˜10%). Herceptin is an antibody-based therapeutic that binds to and blocks Her2/neu receptor and has been shown to be effective against tumors that over-express this receptor. Gleevec® is a drug that treats chronic myeloid leukemia (CML). The drug inhibits the tyrosine kinase BCR-ABL, which is constitutively active in this type of cancer cell and initiates a cell growth signal. Blocking BCR-ABL and intercepting the growth signal halts proliferation; the lack of cell proliferation then induces the programmed cell death called apoptosis. Because this drug works on a target molecule that is aberrant in cancer cells, it has a very high cure rate and few if any side effects. Unfortunately, the mechanism that goes awry in CML represents only a small percentage of human cancers.

However, these results demonstrate that therapies that target specific molecules that are involved in the progression of cancer are more effective than earlier therapies that simply inhibit broad-spectrum cell growth. The new generation of cancer drugs such as Herceptin and Gleevec and others being developed are called “smart” drugs because they home in on and disable specific molecules that are involved in cancer, or more often a particular type of cancer. Thus, in order to effectively determine which therapies are best for a particular patient, the patient's cancer can be characterized at the molecular level and treatments that act on specific aberrant molecules determined by the characterization can then be administered. Failure to characterize a cancer according to molecular signatures prior to treatment could cause the patient more harm than good. For example, by treating a patient with a drug that targets a particular molecule that is aberrant in some cancers, but not the type of cancer that the patient presents with, would constitute withholding appropriate treatment from that patient.

The MUC1 receptor is aberrantly expressed in a number of cancer types. MUC1 is a transmembrane glycoprotein found on the surface of epithelial cells. It has been reported that an estimated 75% of all human solid tumors aberrantly express the MUC1 receptor, including more than 90% of breast cancers and approximately 50% of prostate cancers. Other cancers in which the MUC1 receptor is aberrantly expressed include ovarian, colorectal, pancreatic, some lung cancers, and several others. For some time it has been known that in a healthy cell, the MUC1 receptors are clustered at the apical border, while in cancer cells, it appears to be uniformly expressed over the entire cell surface. This loss of clustering has been correlated to degree of cancer aggressiveness and patient prognosis. It is also known that the MUC1 receptor can be cleaved and shed from the cell surface. Shed receptor can be detected in the blood of healthy patients as well as breast cancer patients. Pregnant or lactating women have higher shed MUC1 levels in serum, while non-pregnant women, regardless of previous pregnancies, have shed MUC1 present in the serum, but at significantly lower levels. Elevated levels of shed MUC1 are only present in small percentage of patients with localized disease (Stage I). As a general rule, MUC1 shedding occurs more frequently as the cancer increases in stage, becoming metastatic. Tests that assess the serum levels of shed MUC1 are approved by the FDA for the detection of breast cancer recurrence in patients initially diagnosed with Stage II or III breast cancer. These tests utilize an antibody that recognizes the terminal repeat units of the MUC1 receptor. The number of tandem repeats of the MUC1 receptor varies from person to person and is not correlated to cancer. However, because a diagnostic test or tracking test must detect elevated levels of shed MUC1, the variable number of antibody epitopes makes it impossible to discriminate between elevated levels and increased antibody binding because that person's MUC1 contains a greater number of tandem repeat units. This variability in the number of repeat units from person to person introduces variability into the test and thus limits its utility for tracking a patient's response to therapy and prevents its use as a diagnostic. Therefore, what is needed is either an antibody that recognizes an epitope that is expressed a single time on each shed portion of the MUC1 receptor, or an antibody that recognizes an epitope that is present on shed MUC1 when cancer is present but not when MUC1 is shed in the normal state.

Proteases comprise another category of proteins that pharmaceutical companies are investigating as therapeutic targets. For example, protease inhibitors are effective treatments for HIV. Metalloproteases have been suggested as therapeutic targets of interest for a variety of conditions, including but not limited to cancers. Metzincins are a super-family of metalloproteins that includes three families of metalloproteases: MMPs, ADAMs, and ADAMTSs (ADAMS which contain one or more thrombospondin (TS) domains). These cleavage enzymes are produced as zymogens, which are not proteolytically active until a pro-peptide or pro-domain is cleaved or removed from its surface. This final processing step typically takes place at the cell surface. However, a subset of the metzincins are cleaved to generate the active enzyme in the golgi by furin or a furin-like enzyme. TIMPs (tissue inhibitors of metalloproteinases) are small proteins that bind to some metalloproteases and inhibit their proteolytic activity.

MMPs (matrix metalloproteinases) are a class of zinc-dependent endoproteases, wherein the metal is required for its activity. Six membrane-tethered MMPs, called MT-MMPs have been identified: MT1-MMP, MT2-MMP, MT3-MMP, MT4-MMP, MT5-MMP and MT6-MMP. All the MT-MMPs are processed to the proteolytically active form by furin. The MMPs were first named for their ability to degrade components of the extracellular matrix. Now, however, MMPs as well as other metalloproteases are emerging as a class of therapeutic targets for the treatment inflammatory diseases and cancer. ADAM-17 is currently of pharmaceutical interest for the treatment of rheumatoid arthritis because it is required for the production of soluble TNFα.

Thus, there is a need to develop new and more accurate molecular signatures of cancers, develop diagnostic methods for characterizing cancers based on these signatures, and develop new therapeutic agents that act on those molecules that are specific to a type of cancer.

### SUMMARY OF THE INVENTION

Certain embodiments of the present invention relate to compositions that are able to inhibit MUC1-related proliferative diseases, particularly cancers, involving inhibiting the portion of MUC1 that functions as a Growth Factor Receptor, cleavage of the full-length receptor to its tumorigenic form or interaction of the MUC1 receptor with its ligands, and methods for treating patients displaying symptoms of, or susceptible to MUC1-associated cancers by either inhibiting direct interactions or by inhibiting their expression. The subject matter of this application involves, in some cases, interrelated products, alternative solutions to a particular problem, and/or a plurality of different uses of a single system or article.

Several methods are disclosed herein of administering to a subject a composition for prevention or treatment of a particular condition. It is to be understood that in each such aspect of the invention, the invention specifically includes the composition for use in the treatment or prevention of that particular condition, as well as use of the composition for the manufacture of a medicament for the treatment or prevention of that particular condition. In some aspects of the invention, the invention also includes a pharmaceutically acceptable carrier.

The present invention includes methods of treatment of selected groups of patients. It is to be understood that all compositions described herein are useful or potentially useful for each described method.

Also included in certain embodiments of the present invention is a combinatorial approach in which structural features identified as characteristic of compositions effective for treatment at various disease stages are used as the basis for combinatorial synthesis of a wide variety of structural homologs, analogs, derivatives, enantiomers and functionally equivalent compositions thereof, for identification of a wide variety of compositions useful for treatment of MUC1-associated cancers. Thus, in one embodiment, the invention involves providing any one or more of compositions 1-188, performing a combinatorial synthesis resulting in a plurality of compositions. Then, one can perform an assay involving the plurality of the compositions to determine their effectiveness in cancer treatment, specifically, for example, treatment of cancers disclosed herein. Compositions 1-188 also can be altered using medicinal chemistry techniques.

Another aspect of the invention provides, in certain embodiments, a pharmaceutical preparation comprising a composition comprising any of the compositions 1-188, and a pharmaceutically active carrier, including carbohydrates, lectins and/or lectin receptors. In one embodiment, compositions can comprise homologs, analogs, derivatives, enantiomers and functionally equivalent compositions thereof of compositions 1-188. In all structures herein, atom locations, if unlabeled, are carbon with appropriate hydrogen(s). The invention also provides, in certain embodiments, a method involving promoting the prevention or treatment of MUC1-associated cancer via administration of any one or more of the compositions of the present invention and/or homologs, analogs, derivatives, enantiomers and functionally equivalent compositions thereof.

In another aspect, the invention provides a kit including any one or more of the compositions of the present invention and/or homologs, analogs, derivatives, enantiomers and functionally equivalent compositions thereof; and instructions for use of these compositions for treatment of cancer characterized by aberrant expression of MUC1.

In one aspect, the invention is defined, at least in part, by a method. In some embodiments of the invention, the method involves treating a human patient susceptible to or exhibiting symptoms of a cancer characterized by aberrant expression of MUC1 with any of the compositions disclosed herein. In one set of embodiments, the patient is susceptible to, but does not exhibit symptoms of, cancer characterized by aberrant expression of MUC1. In another set of embodiments, the patient exhibits symptoms of cancer characterized by aberrant expression of MUC1. In some embodiments of the method, the patient is not otherwise indicated for treatment for a cancer characterized by aberrant expression of a hedgehog protein.

In another aspect, the invention is directed to a method of making any of the embodiments described herein. In yet another aspect, the invention is directed to a method of using any of the embodiments described herein.

In one aspect, the present invention is directed to a method for treating or preventing cancer in a subject comprising administering a treatment effective amount of a compound belonging to Formulae I, II, III, IV, V, VI, VII, VIII, IX, X, XI, or XII to a subject in need thereof. Formulae I, II, III, V, V, VI, VII, VIII, IX, X, XI, or XII are described in the present application. Further, the invention is directed to the discovery of Compounds Nos. 1 through 188 as set forth in Tables 2 to 5. In particular, the invention is directed to a compound belonging to Formulae I, II, III, IV, V, VI, VII, or VIII, which include without limitation Compound No. 1-5, 7, 11, 13-15, 17-24, 26, 28-31, 42-48, 51, 55-106, 173, 174-179, 184, 185, or 186. In particular, Compound No. 28, 173, 184, or 185 is preferred.

The invention is also directed to a compound belonging to Formula IX such as Compound No. 33, 50, 166-172, 180-183, or 188. In particular, Compound No. 182 or 188 is preferred.

The invention is also directed to a compound belonging to Formula X such as Compound No. 8, 25, 115, 118, 120, 122-128, 130-132, or 134. In particular, Compound No. 118 or 125 is preferred.

The invention is also directed to a compound belonging to Formula XI such as Compound No. 35, 107-113, or 114. In particular, Compound No. 107 or 109 is preferred.

The invention is also directed to a compound belonging to Formula XII such as Compound No. 6, 9, 10, 12, 16, 27, 32, 34, 36-41, 49, 52-54, 116, 117, 119, 121, 129, 133, 135-165, or 187.

The invention is also directed to a method of treating or preventing cancer as described above, which further includes (i) testing a bodily sample from the subject for aberrant expression of MUC1; and (ii) treating the subject in need of the compound with the compound. Preferably, the compound belongs to Formulae I, II, III, IV, V, VI, VII, or VIII. Alternatively, the compound belongs to Formulae IX, X, XI, or XII.

Aberrant expression of MUC1 can be observed by loss of clustering pattern of MUC1 on cell surface; or by membrane staining that uniformly covers a cell when contacted with anti-nat-PSMGFR or anti-var-PSMGFR peptide.

In another aspect, the invention is directed to the use of a compound that has dual moieties: MGFR binding moiety and metal chelating moiety. In this regard, the invention is directed to a method for treating or preventing MUC1-positive cancers comprising: (i) testing a bodily sample for aberrant expression of MUC1; and (ii) treating the patient with a compound comprising a MGFR binding region and metal chelator group wherein the metal is zinc, magnesium or nickel.

The compound may be a metal-dependent protein inhibitor, wherein the metal dependent protein may be a member of the kinesin family, a kenesin spindle protein, or Costal2, or an enzyme that cleaves MUC1 such as matrix metalloprotease, particularly MT1-MMP or MMP-14, Furin, ADAM-17-TASE. The inhibitor may be TIMP, TIMP2 or TIMP3.

Taking advantage of understanding the biochemistry of the state of MUC1 on normal cells versus cancer cells, it is possible to detect cancer cells by assaying for binding of a ligand to the MGFR portion of MUC1 to determine the distribution of the MUC1 on the cell surface. Uniform distribution indicates cancerous cells. Thus, in one aspect, the invention is directed to a method for diagnosing cancer cells, comprising: (i) contacting a population of cells with a MGFR specific ligand bound to a signal generating label; and (ii) assaying for binding of the ligand to MGFR on a membrane, in which presence of uniform signal on the membrane on cell surface when contacted with the ligand indicates that the cells are cancerous.

These and other objects of the invention will be more fully understood from the following description of the invention, the referenced drawings attached hereto and the claims appended hereto.

### BRIEF DESCRIPTION OF THE DRAWINGS

The present invention will become more fully understood from the detailed description given herein below, and the accompanying drawings which are given by way of illustration only, and thus are not limitative of the present invention, and wherein;

FIG. 1 shows a western blot that shows that an inventive antibody anti-PSMGFR specifically recognizes a low molecular weight MUC1 cleavage product from breast tumor cell lines.

FIG. 2 shows a western blot that shows that the inventive antibody anti-PSMGFR recognizes a low molecular weight MUC1 cleavage product from breast tumor cells and from cells into which a MUC1 variant that terminates at the end of PSMGFR has been transfected (MUC1* refers to HEK 293s transfected with nat-PSMGFR).

FIG. 3A-3D show photos of fluorescence microscopy of HEK 293 cells transfected with either the PSMGFR portion of MUC1 (A and B) or the full MUC1 receptor (C and D). FIGS. 3A and C show green fluorescence from GFP that was carried on the transfection plasmid and red fluorescence from the antibody that recognizes the PSMGFR portion. B and D show red fluorescence alone that comes from the antibody that recognizes the PSMGFR portion. Results show that the antibody is able to recognize the PSMGFR site on both transfectants, albeit at a greatly diminished capacity in the full-length clone due to receptor clustering.

FIG. 4A-4D show photos of cancerous MUC1-positive (A and C) and MUC1-negative (B and D) breast (A and B) and lung (C and D) tissue specimens that have been stained with anti-var-PSMGFR.

FIG. 5A-5D show photos of cancerous MUC1-positive (A and C) and MUC1-negative (B and D) breast tissue specimens that have been stained with either anti-var-PSMGFR (A and B) or VU4H5 (C and D) an antibody that binds to the MUC1 tandem repeat units.

FIG. 6A-6D show photos of cancerous MUC1-positive (A and C) and MUC1-negative (B and D) lung tissue specimens that have been stained with either anti-var-PSMGFR (A and B) or VU4H5 (C and D) an antibody that binds to the MUC1 tandem repeat units.

FIG. 7A-7B show photos of contiguous MUC1-negative cancerous breast tissue specimens that have been stained with either anti-var-PSMGFR (A) or VU4H5 (B) an antibody that binds to the MUC1 tandem repeat units and show that the tandem repeat antibody stains necrotic tissue of a MUC1-negative specimen.

FIG. 8A-8D show photos of prostate cancer specimens (A and C) and benign prostatic hyperplasia specimens (B and D) that have been stained with either anti-var-PSMGFR (A and B) or VU4H5 (C and D) an antibody that binds to the MUC1 tandem repeat units and show that the tandem repeat antibody does not stain the ducts of cancerous prostate tissue specimens and that neither antibody stained the benign specimens.

FIG. 9A-9D show photos of colon cancer specimens (A and C) and normal colon specimens (B and D) that have been stained with either anti-var-PSMGFR (A and B) or VU4H5 (C and D) an antibody that binds to the MUC1 tandem repeat units and show that the tandem repeat antibody does not stain the most cancerous portions of the specimen.

FIG. 10A-10C show photos of tissue specimens wherein displastic portions of cancerous breast tumor specimens that were stained with either H & E (A), anti-var-PSMGFR (B) or VU4H5 (C) and show that staining with anti-var-PSMGFR (B) renders visible distinct rings inside of the displastic cells.

FIG. 11 shows a mass spectroscopy spectrum of the PSMGFR portion of the MUC1 receptor.

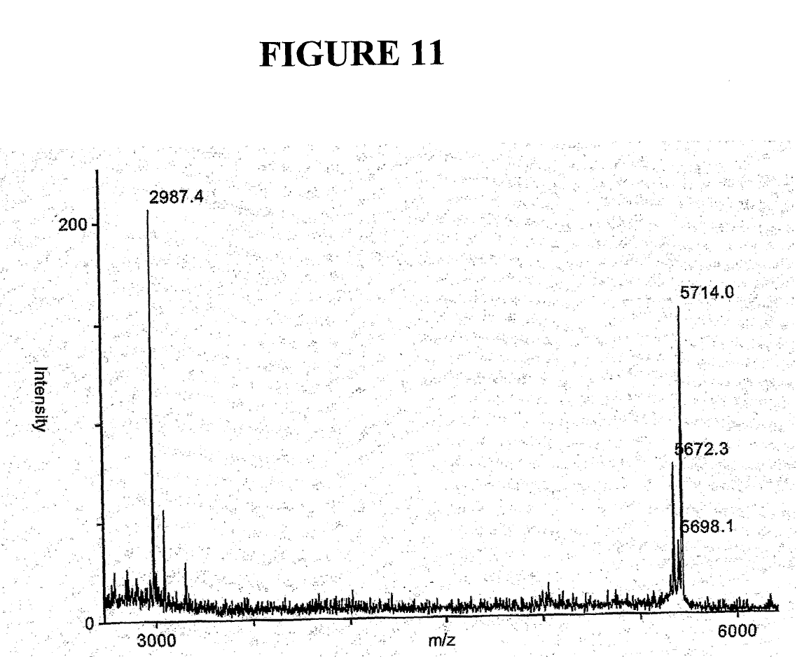

FIG. 12 shows a MALDI Mass Spectrum of Compound No. 173—PSMGFR peptide Complex in Ammonium Phosphate Buffer.

FIG. 13 shows a MALDI Mass Spectrum of Compound No. 173—PSMGFR peptide Complex in Ammonium Chloride Buffer.

FIG. 14 shows a MALDI Mass Spectrum of Compound No. 173—PSMGFR peptide Complex in Ammonium Nitrate Buffer.

FIG. 15 shows a graph that plots the growth of compositions of the invention against MUC1-positive versus MUC1-negative tumor cells.

FIG. 16 shows that Compound No. 173, 28, and 118 all similarly inhibit the growth of breast tumor cell line ZR-75-1.

FIG. 17 shows that Compound No. 173 Blocks Growth of MUC1 Transfected Cells; Not Parent Cells. FLR #8, #35 are PSMGFR transfectant; Muc1 #1, #28 are full-length transfectants shown to actually be cleaved to the MGFR.

FIG. 18 shows that Compound No. 28 Blocks Growth of MUC1 Transfected Cells; Not Parent Cells. FLR #8, #35 are PSMGFR transfectant; Muc1 #1, #28 are full-length transfectants shown to actually be cleaved to the MGFR.

FIG. 19 shows that Compound 118 Blocks Growth of MUC1 Transfected HEK 293 Cells; Not Parent Cells or cells transfected with empty vector.

FIG. 20 shows that Compound No. 125 Blocks Growth of MUC1 Transfected HEK 293 Cells; Not Parent Cells.

FIG. 21 shows that Compound No. 188 Blocks Growth of MUC1 Transfected Cells; Not Parent Cells.

FIG. 22 shows that Compound No. 182 Blocks Growth of MUC1 Transfected Cells; Not Parent Cells.

FIG. 23 shows a mass spec of Compound No. 173 chelating Mg.

FIG. 24 shows a mass spec that shows that Compound No. 186 does not chelate metals.

FIG. 25 shows a mass spec that shows that Compound No. 173 chelates Zn.

FIG. 26 shows a mass spec that shows that Compound No. 28 chelates Zn.

FIG. 27 shows a mass spec that shows that Compound No. 185 chelates Zn.

FIG. 28 shows a western blot of conditioned media around T47D cells treated with compound or DMSO alone that shows that Compound No. 173, 184, and 28 chelate Zn and inhibit cleavage of MUC1 to the tumor specific form consisting essentially of PSMGFR.

FIG. 29 shows a graph that plots the growth of MUC1 transfected cells compared to the parent cell line and shows that both the non-chelator compound and the chelating analog both inhibit the growth of MUC1-positive cells.

### DETAILED DESCRIPTION OF THE INVENTION

### Definitions

The term “MUC1 Growth Factor Receptor” (MGFR) is a functional definition meaning that portion of the MUC1 receptor that interacts with an activating ligand, such as a growth factor or a modifying enzyme such as a cleavage enzyme, to promote cell proliferation. The MGFR region of MUC1 is that extracellular portion that is closest to the cell surface and contains most or all of the PSMGFR, as defined below. The MGFR is inclusive of both unmodified peptides and peptides that have undergone enzyme modifications, such as, for example, phosphorylation, glycosylation, etc. Results of the invention are consistent with a mechanism in which the MGFR is the portion of the receptor that remains attached to the cell surface after receptor cleavage or shedding whereupon the MGFR is made more accessible to activating ligand(s) upon MUC1 cleavage.

The term “Interchain Binding Region” (IBR) is a functional definition meaning that portion of the MUC1 receptor that binds strongly to identical regions of other MUC1 molecules giving MUC1 the ability to aggregate (i.e. self-aggregate) with other MUC1 receptors via the IBRs of the respective receptors. This self-aggregation may contribute to MUC1 receptor clustering, observed in healthy cells.

In a preferred embodiment, the IBR may be approximately defined as a stretch of at least 12 to 18 amino acid sequence within the region of the full-length human MUC1 receptor defined as comprising amino acids 507 to 549 of the extracellular sequence of the MUC1 receptor (SEQ ID NO: 10), with amino acids 525 through 540 and 525 through 549 especially preferred (numbers refer to Andrew Spicer et al., J. Biol. Chem Vol 266 No. 23, 1991 pgs. 15099-15109; these amino acid numbers correspond to numbers 1067, 1109, 1085, 1100, 1085, 1109 of Genbank accession number P15941; PID G547937, SEQ ID NO: 10) or fragments, functional variants or conservative substitutions thereof, as defined in more detail below.

The term “cleaved IBR” means the IBR (or a portion thereof) that has been released from the receptor molecule segment which remains attached to the cell surface. The release may be due to enzymatic or other cleavage of the IBR. As used herein, when the IBR is “at the surface of a cell”, it means the IBR is attached to the portion of the cell surface receptor that has not been shed, or cleaved.

The term “Constant Region” (CR) is any non-repeating sequence of MUC1 that exists in a 1:1 ratio with the IBR and forms part of the portion of MUC1 that is shed upon cleavage in healthy and tumorigenesic cells.

The term “Repeats” is given its normal meaning in the art.

The term “Primary Sequence of the MUC1 Growth Factor Receptor” (PSMGFR) is a peptide sequence that defines most or all of the MGFR in some cases, and functional variants and fragments of the peptide sequence, as defined below. The PSMGFR is defined as SEQ ID NO: 13 listed below in Table 1, and all functional variants and fragments thereof having any integer value of amino acid substitutions up to 20 (i.e. 1, 2, 3, 4, 5, 6, 7, 8, 9, 10, 11, 12, 13, 14, 15, 16, 17, 18, 19, or 20) and/or any integer value of amino acid additions up to 27 or deletions up to 9 at its N-terminus. A “functional variant or fragment” in the above context refers to such variant or fragment having the ability to specifically bind to, or otherwise specifically interact with, ligands that specifically bind to, or otherwise specifically interact with, the peptide of SEQ ID NO: 13, while not binding strongly to identical regions of other peptide molecules identical to themselves, such that the peptide molecules would have the ability to aggregate (i.e. self-aggregate) with other identical peptide molecules. One example of a PSMGFR that is a functional variant of the PSMGFR peptide of SEQ NO: 13 (referred to as nat-PSMGFR—for “native”) is SEQ NO: 7 (referred to as var-PSMGFR, which differs from nat-PSMGFR by including an −SPY-sequence instead of the native −SRY—(see bold text in sequence listings)). Var-PSMGFR may have enhanced conformational stability, when compared to the native form, which may be important for certain applications such as for antibody production. The PSMGFR is inclusive of both unmodified peptides and peptides that have undergone enzyme modifications, such as, for example, phosphorylation, glycosylation, etc. A histidine-tagged PSMGFR (e.g. See Table 1—SEQ ID NO: 2) is abbreviated herein as His-PSMGFR.

The term “Extended Sequence of the MUC1 Growth Factor Receptor” (ESMGFR) is a peptide sequence, defined below (See Table 1—SEQ ID NO: 3), that defines all of His-var-PSMGFR plus 9 amino acids of the proximal end of PSIBR and that defines one of the MUC1 cleavage products found in tumor cells that remains attached to the cell surface and is able to interact with activating ligands in a manner similar to the PSMGFR.

The term “Tumor-Specific Extended Sequence of the MUC1 Growth Factor Receptor” (TSESMGFR) is a peptide sequence (See, as an example, Table 1—SEQ ID NO: 28) that defines a MUC1 cleavage product found in tumor cells that remains attached to the cell surface and is able to interact with activating ligands in a manner similar to the PSMGFR.

PSIBR is a peptide sequence, defined below (See Table 1—SEQ ID NO: 8), that defines most or all of the IBR.

“Truncated Interchain Binding Region” (TPSIBR) is a peptide sequence defined below (See Table 1—SEQ ID NO: 27), that defines a smaller portion of the IBR that is released from the cell surface after receptor cleavage in some tumor cells.

PSMGFRTC is a truncated MUC1 receptor isoform comprising PSMGFR and at or within about up to 30 (i.e. within 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30) amino acids of its N-terminus and comprising the transmembrane and cytoplasmic sequences of full-length MUC1 receptor.

As used herein, the phrase “at its N-terminus” referring to the location of a recited sequence within a larger molecule, such as a polypeptide or receptor, refers to such a sequence being no more than 30 amino acids from the N-terminal amino acid of the molecule. Optionally the PSMGFRTC, as well as the other truncated MUC1 receptor isoforms discussed below, can include a MUC1 N-terminal signaling sequence (Table 1—SEQ ID NO: 19, 20, or 21), typically between 20 and 30 amino acids in length, or a functional fragment or variant thereof. Such a sequence is typically encoded by the nucleic acid constructs encoding the truncated MUC1 receptor isoform and is translated but is typically cleaved prior to or upon insertion of the receptor in the membrane of the cell. Such a PSMGFRTC, i.e. including the optional signal sequence, would still be a peptide or protein “having a PSMGFR” sequence “at its N-terminus” by the above definition. An example is nat-PSMGFRTC (SEQ ID NO: 14, with or without the signal peptide of SEQ ID NO: 19, 20, or 21 at the extreme N-terminus) having nat-PSMGFR (SEQ NO: 3613) at its N-terminus (i.e. at the extreme N-terminal end or within 30 amino acids thereof).

The term “separation” means physical separation from a cell, i.e. a situation in which a portion of MUC1 that was immobilized with respect to a cell is no longer immobilized with respect to that cell. E.g. in the case of cleavage of a portion of MUC1, the portion that is cleaved is “separated” if it is free to migrate away from the cell and thereafter may be detected in a bodily fluid, or immobilized at a location remote from the cell from which it was cleaved such as another cell, a lymph node, etc.

The term “binding” refers to the interaction between a corresponding pair of molecules that exhibit mutual affinity or binding capacity, typically specific or non-specific binding or interaction, including biochemical, physiological, and/or pharmaceutical interactions. Biological binding defines a type of interaction that occurs between pairs of molecules including proteins, nucleic acids, glycoproteins, carbohydrates, hormones and the like. Specific examples include antibody/antigen, antibody/hapten, enzyme/substrate, enzyme/inhibitor, enzyme/cofactor, binding protein/substrate, carrier protein/substrate, lectin/carbohydrate, receptor/hormone, receptor/effector, complementary strands of nucleic acid, protein/nucleic acid repressor/inducer, ligand/cell surface receptor, virus/ligand, etc.

The term “binding partner” refers to a molecule that can undergo binding with a particular molecule. Biological binding partners are examples. For example, Protein A is a binding partner of the biological molecule IgG, and vice versa.

The term “aggregate” (noun) means a plurality of cell surface receptors or fragments thereof (e.g. MUC1) immobilized with respect to each other with or without an intermediate auxiliary to the host system. This includes self-aggregation of healthy receptors at a cell surface; self-aggregation of cleaved receptors or fragments bound to each other; cleaved receptors or fragments bound to receptors or fragments attached to a cell surface; receptors or fragments, whether attached to a cell or cleaved, immobilized with respect to each other via an intermediate auxiliary to the host. “Intermediate auxiliary to the host system” includes a synthetic species such as a polymer, dendrimer, etc., or a naturally-occurring species, for example an IgM antibody, which is not simply naturally present in the host system but is added to the host system from a source external to the host system. This excludes aggregation that is the result of an intermediate naturally present in the host system such as a growth factor that can cause disease-associated aggregation (“Inductive multimerization”). “Aggregate” (verb) or “aggregation” means the process of forming an aggregate (noun).

“Inductive multimerization” refers to aggregation wherein the aggregate formed can act to induce the cells to grow or proliferate. Inductive multimerization typically involves dimerization or tetramerization of cell surface receptors, for example by a growth factor or other activating ligand, but can also involve higher order multimerization, so long as the degree of multimerization is not so great as to mimic natural receptor clustering, in a particular cell type, which prevents receptors from signaling the cell to grow or proliferate.

“Preventative clustering” refers to multimerization of receptors to form an aggregate involving a sufficient number of receptors to mimic natural receptor clustering, in a particular cell type, which prevents receptors from signaling the cell to grow or proliferate, for example with an intermediate auxiliary to the host system.

A “ligand” to a cell surface receptor, refers to any substance that can interact with the receptor to temporarily or permanently alter its structure and/or function. Examples include, but are not limited to binding partners of the receptor, (e.g. antibodies or antigen-binding fragments thereof), and agents able to alter the chemical structure of the receptor (e.g. modifying enzymes).

An “activating ligand” refers to a ligand able to interact with a receptor to transduce a signal to the cell. Activating ligands can include, but are not limited to, species that effect inductive multimerization of cell surface receptors such as a single molecular species with greater than one active site able to bind to a receptor; a dimer, a tetramer, a higher multimer, a bivalent antibody or bivalent antigen-binding fragment thereof, or a complex comprising a plurality of molecular species. Activating ligands can also include species that modify the receptor such that the receptor then transmits a signal. Enzymes can also be activating ligands when they modify a receptor to make it a new recognition site for other activating ligands, e.g. glycosylases are activating ligands when the addition of carbohydrates enhances the affinity of a ligand for the receptor. Cleavage enzymes are activating ligands when the cleavage product is the more active form of the receptor, e.g. by making a recognition site for a ligand more accessible. In the context of MUC1 tumor cells, an activating ligand can be a species that cleaves MUC1, chemically modifies the receptor, or species that interact with the MGFRs on the surface of the MUC1 tumor cells to transduce a signal to the cell that stimulates proliferation, e.g. a species that effects inductive multimerization.

A “growth factor” refers to a species that may or may not fall into a class of previously-identified growth factors, but which acts as a growth factor in that it acts as an activating ligand.

A “MUC1 presenting cell” refers to both non-cancerous and cancerous cells expressing MUC1 and/or MGFRs on the surface. A “MUC1 tumor cell” or “MUC1 cancer cell” or “cancerous MUC1 cell” or a MUC1-positive cancer refers to a cancerous cell that aberrantly expresses MUC1 and/or MGFR on its surface.

“Colloids”, as used herein, means nanoparticles, i.e. very small, self-suspendable or fluid-suspendable particles including those made of material that is, e.g., inorganic or organic, polymeric, ceramic, semiconductor, metallic (e.g. gold), non-metallic, crystalline, amorphous, or a combination. Typically, colloid particles used in accordance with the invention are of less than 250 nm cross section in any dimension, more typically less than 100 nm cross section in any dimension, and in most cases are of about 2-30 nm cross section. One class of colloids suitable for use in the invention is 10-30 nm in cross section, and another about 2-10 nm in cross section. As used herein this term includes the definition commonly used in the field of biochemistry.

As used herein, a component that is “immobilized relative to” another component either is fastened to the other component or is indirectly fastened to the other component, e.g., by being fastened to a third component to which the other component also is fastened, or otherwise is transitionally associated with the other component. For example, a signaling entity is immobilized with respect to a binding species if the signaling entity is fastened to the binding species, is fastened to a colloid particle to which the binding species is fastened, is fastened to a dendrimer or polymer to which the binding species is fastened, etc. A colloid particle is immobilized relative to another colloid particle if a species fastened to the surface of the first colloid particle attaches to an entity, and a species on the surface of the second colloid particle attaches to the same entity, where the entity can be a single entity, a complex entity of multiple species, a cell, another particle, etc.

“Signaling entity” means an entity that is capable of indicating its existence in a particular sample or at a particular location. Signaling entities of the invention can be those that are identifiable by the unaided human eye, those that may be invisible in isolation but may be detectable by the unaided human eye if in sufficient quantity (e.g., colloid particles), entities that absorb or emit electromagnetic radiation at a level or within a wavelength range such that they can be readily detected visibly (unaided or with a microscope including an electron microscope or the like), or spectroscopically, entities that can be detected electronically or electrochemically, such as redox-active molecules exhibiting a characteristic oxidation/reduction pattern upon exposure to appropriate activation energy (“electronic signaling entities”), or the like. Examples include dyes, pigments, electroactive molecules such as redox-active molecules, fluorescent moieties (including, by definition, phosphorescent moieties), up-regulating phosphors, chemiluminescent entities, electrochemiluminescent entities, or enzyme-linked signaling moieties including horseradish peroxidase and alkaline phosphatase. “Precursors of signaling entities” are entities that by themselves may not have signaling capability but, upon chemical, electrochemical, electrical, magnetic, or physical interaction with another species, become signaling entities. An example includes a chromophore having the ability to emit radiation within a particular, detectable wavelength only upon chemical interaction with another molecule. Precursors of signaling entities are distinguishable from, but are included within the definition of, “signaling entities” as used herein.

As used herein, “fastened to or adapted to be fastened”, in the context of a species relative to another species or to a surface of an article, means that the species is chemically or biochemically linked via covalent attachment, attachment via specific biological binding (e.g., biotin/streptavidin), coordinative bonding such as chelate/metal binding, or the like. For example, “fastened” in this context includes multiple chemical linkages, multiple chemical/biological linkages, etc., including, but not limited to, a binding species such as a peptide synthesized on a polystyrene bead, a binding species specifically biologically coupled to an antibody which is bound to a protein such as protein A, which is attached to a bead, a binding species that forms a part (via genetic engineering) of a molecule such as GST or Phage, which in turn is specifically biologically bound to a binding partner covalently fastened to a surface (e.g., glutathione in the case of GST), etc. As another example, a moiety covalently linked to a thiol is adapted to be fastened to a gold surface since thiols bind gold covalently. Similarly, a species carrying a metal binding tag is adapted to be fastened to a surface that carries a molecule covalently attached to the surface (such as thiol/gold binding) which molecule also presents a chelate coordinating a metal. A species also is adapted to be fastened to a surface if a surface carries a particular nucleotide sequence, and the species includes a complementary nucleotide sequence.

“Covalently fastened” means fastened via nothing other than one or more covalent bonds. E.g. a species that is covalently coupled, via EDC/NHS chemistry, to a carboxylate-presenting alkyl thiol which is in turn fastened to a gold surface, is covalently fastened to that surface.

“Specifically fastened” or “adapted to be specifically fastened” means a species is chemically or biochemically linked to another specimen or to a surface as described above with respect to the definition of “fastened to or adapted to be fastened”, but excluding all non-specific binding.

Certain embodiments of the invention make use of self-assembled monolayers (SAMs) on surfaces, such as surfaces of colloid particles, and articles such as colloid particles having surfaces coated with SAMs. In one set of embodiments, SAMs formed completely of synthetic molecules completely cover a surface or a region of a surface, e.g. completely cover the surface of a colloid particle. “Synthetic molecule”, in this context, means a molecule that is not naturally occurring, rather, one synthesized under the direction of human or human-created or human-directed control. “Completely cover” in this context, means that there is no portion of the surface or region that directly contacts a protein, antibody, or other species that prevents complete, direct coverage with the SAM. I.e. in certain embodiments the surface or region includes, across its entirety, a SAM consisting completely of non-naturally-occurring molecules (i.e. synthetic molecules). The SAM can be made up completely of SAM-forming species that form close-packed SAMs at surfaces, or these species in combination with molecular wires or other species able to promote electronic communication through the SAM (including defect-promoting species able to participate in a SAM), or other species able to participate in a SAM, and any combination of these. Preferably, all of the species that participate in the SAM include a functionality that binds, optionally covalently, to the surface, such as a thiol which will bind to a gold surface covalently. A self-assembled monolayer on a surface, in accordance with certain embodiments of the invention, can be comprised of a mixture of species (e.g. thiol species when gold is the surface) that can present (expose) essentially any chemical or biological functionality. For example, they can include tri-ethylene glycol-terminated species (e.g. tri-ethylene glycol-terminated thiols) to resist non-specific adsorption, and other species (e.g. thiols) terminating in a binding partner of an affinity tag, e.g. terminating in a chelate that can coordinate a metal such as nitrilotriacetic acid which, when in complex with nickel atoms, captures a metal binding tagged-species such as a histidine-tagged binding species. Also disclosed is a method for rigorously controlling the concentration of essentially any chemical or biological species presented on a colloid surface or any other surface. Without this rigorous control over peptide density on each colloid particle, co-immobilized peptides would readily aggregate with each other to form micro-hydrophobic-domains that would catalyze colloid-colloid aggregation in the absence of aggregate-forming species present in a sample. This is an advantage of certain embodiments of the present invention, over existing colloid agglutination assays. In many embodiments of the invention the self-assembled monolayer is formed on gold colloid particles.

The kits described herein, contain one or more containers, which can contain compounds such as the species, signaling entities, biomolecules, and/or particles as described. The kits also may contain instructions for mixing, diluting, and/or administrating the compounds. The kits also can include other containers with one or more solvents, surfactants, preservative and/or diluents (e.g. normal saline (0.9% NaCl, or 5% dextrose) as well as containers for mixing, diluting or administering the components to the sample or to the patient in need of such treatment.

The compounds in the kit may be provided as liquid solutions or as dried powders. When the compound provided is a dry powder, the powder may be reconstituted by the addition of a suitable solvent, which also may be provided. Liquid forms of the compounds may be concentrated or ready to use. The solvent will depend on the compound and the mode of use or administration. Suitable solvents for are well known for drug compounds and are available in the literature.

The term “cancer”, as used herein, may include but is not limited to: biliary tract cancer; bladder cancer; brain cancer including glioblastomas and medulloblastomas; breast cancer; cervical cancer; choriocarcinoma; colon cancer; endometrial cancer; esophageal cancer; gastric cancer; hematological neoplasms including acute lymphocytic and myelogenous leukemia; multiple myeloma; AIDS-associated leukemias and adult T-cell leukemia lymphoma; intraepithelial neoplasms including Bowen's disease and Paget's disease; liver cancer; lung cancer; lymphomas including Hodgkin's disease and lymphocytic lymphomas; neuroblastomas; oral cancer including squamous cell carcinoma; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, and osteosarcoma; skin cancer including melanoma, Kaposi's sarcoma, basocellular cancer, and squamous cell cancer; testicular cancer including germinal tumors such as seminoma, non-seminoma (teratomas, choriocarcinomas), stromal tumors, and germ cell tumors; thyroid cancer including thyroid adenocarcinoma and medullar carcinoma; and renal cancer including adenocarcinoma and Wilms tumor. Preferred cancers are; breast, prostate, lung, ovarian, colorectal, and brain cancer. Neoplasms in benign or malignant form are also considered within the purview of cancerous state.

The term “cancer treatment” as described herein, may include but is not limited to: chemotherapy, radiotherapy, adjuvant therapy, or any combination of the aforementioned methods. Aspects of treatment that may vary include, but are not limited to: dosages, timing of administration, or duration or therapy; and may or may not be combined with other treatments, which may also vary in dosage, timing, or duration. Another treatment for cancer is surgery, which can be utilized either alone or in combination with any of the aforementioned treatment methods. One of ordinary skill in the medical arts may determine an appropriate treatment.

An “agent for prevention of cancer or tumorigenesis” means any agent that counteracts any process associated with cancer or tumorigenesis described herein. For example, an agent that interacts with (e.g. binds to) to MGFR thereby reducing or preventing interaction, with MGFR, of an agent that promotes tumorigenesis by its interaction with MGFR.

An “agent that reduces cleavage of a cell surface receptor interchain binding region” as used herein is any composition that prevents or reduces cleavage of the MUC1 receptor between the MGFR and the N-terminus of the IBR that would otherwise occur in the absence of the agent. Cleavage of the receptor between the MGFR and the N-terminus of the IBR can be caused by activity of enzymes that are membrane-associated or soluble, e.g. matrix metalloproteases (MMPs and MT-MMPs). Some of these enzymes are directly responsible for cleavage. Other enzymes can affect cleavage, (e.g. prevent cleavage at a particular location) by modifying MUC1 with sugar groups or phosphates that mask a recognition epitope associated with cleavage. Other enzymes can promote cleavage at a particular location by modifying MUC1 with sugar groups or phosphates that create a recognition motif for cleavage at that location. Other enzymes can promote cleavage of receptors by activating other cleavage enzymes. One way to select agents that reduce cleavage of a cell surface receptor IBR is to first identify enzymes that affect cleavage as described above, and screen agents, and their analogs, for their ability to alter the activity of those enzymes. Another way is to test agents that are known to affect the activity of similar enzymes (e.g. from the same family) for their ability to alter the site of cleavage of MUC1, and to similarly test analogs of these agents. Alternatively, agents are screened in a cell-free assay containing the enzyme and MUC1 receptors, and the rate or position of cleavage measured by antibody probing, Polymerase Chain Reaction (PCR), or the like. Alternatively, without first identifying enzymes that affect MUC1, agents are screened against cells that present MUC1 for the agents' ability to alter cleavage site or the rate of cleavage of MUC1. For example, agents can be screened in an assay containing whole cells that present MUC1 and aggregation potential of the cell supernatant can be measured, an indication of the amount of IBR that remains attached to the cleaved portion of MUC1, i.e. the degree of cleavage between MGFR and IBR. In another technique, agents can be screened in an assay containing whole cells that present MUC1, the supernatant removed, and the cell remain tested for accessibility of the MGFR portion, e.g. using a labeled antibody to the MGFR. Agents can be identified from commercially available sources such as molecular libraries, or rationally designed based on known agents having the same functional capacity and tested for activity using the screening assays.

An “agent that reduces cleavage of the MUC1 receptor” is any composition that prevents or reduces cleavage of the MUC1 receptor at any location. Such an agent can be used to treat a subject having cancer or at risk for developing cancer because if cleavage is prevented, then the accessibility of the MGFR, a functional receptor associated with cancer, is reduced or prevented. Such agents can be selected by exposing cells to a candidate agent and determine, in the supernatant, the amount of cleaved MUC1 receptor, relative to a control.

A subject, as used herein, refers to any mammal (preferably, a human), and preferably a mammal that may be susceptible to tumorigenesis or cancer associated with the abherrant expression of MUC1. Examples include a human, non-human primate, cow, horse, pig, sheep, goat, dog, or cat. Generally, the invention is directed toward use with humans.

As used herein, “bodily sample” refers to any body tissue or body fluid sample obtained from a subject. Preferred are body fluids, for example lymph, saliva, blood, urine, milk and breast secretions, and the like. Blood is preferred in certain embodiments. Samples of tissue and/or cells for use in the various methods described herein can be obtained through standard methods including, but not limited to: tissue biopsy, including punch biopsy and cell scraping, needle biopsy, and collection of blood or other bodily fluids by aspiration or other methods.

### MUC1 Growth Factor Receptor

The present invention, in certain embodiments, involves compositions and methods for cancer treatment and, in particular, to compositions that are able to inhibit interactions involving the MUC1 Growth Factor Receptor and/or its ligands, and methods for treating patients displaying symptoms of, or susceptible to MUC1-associated cancers. The invention also relates to assays and/or use of such compositions for the treatment of patients susceptible to or exhibiting symptoms characteristic of cancer or tumorigenesis. Other compositions of certain embodiments of the present invention useful for the treatment or prevention of cancer or tumorigenesis include homologs, analogs, derivatives, enantiomers or functional equivalents of compositions disclosed herein. Assays can be performed, according to certain embodiments of the invention, to screen for and identify such compositions, and also for identifying which compositions are effective at various stages of the disease process.

The present invention, in certain embodiments, involves compositions and methods for the treatment or prevention of proliferative disorders, including cancers, and in particular to those proliferative disorders that involve the cell surface receptor MUC1. In another aspect, the invention relates to the discovery of a variety of compositions (e.g., drugs) useful for inhibition of cell proliferation, including proliferation associated with tumors such as MUC1-related tumors. The invention, in certain embodiments, also relates to diagnostic methods for determining whether the proliferative disorder involves MUC1 and then linking these diagnostic methods to determining effective treatments for those conditions that involve MUC1. The invention also relates to diagnostic methods that can then be used to track the progress of patient in response to these treatments. In yet another aspect, the invention relates to the discovery of a variety of compositions (e.g., drugs) useful for inhibition of cell proliferation, including proliferation associated with tumors that involves compositions that can abstract a metal, and in particular can abstract a Mg or Zn cation from metal dependent proteins, such as kinesins and matrix metalloproteases. The compositions of the present invention can be provided in a kit including instructions for use of the composition for treatment of diseases.

MUC1 is a cell surface receptor that is aberrantly expressed in a number of cancers. It has been observed that in a healthy cell the receptor is clustered at the apical border and on a tumor cell, the receptors are distributed over the entire cell surface. It is known that the MUC1 receptor can be cleaved and it is also known that the receptor under some circumstances can shuttle between states of cell surface expression and cellular internalization.

The present invention involves, in certain embodiments, compounds for the treatment or prevention of proliferative diseases including cancers. Particularly described are compounds for the treatment of cancers wherein the cells of the cancer present the cell surface receptor MUC1. Many of these compounds inhibit, either directly or indirectly, the MUC1 receptor and by so doing inhibit the growth of MUC1-positive tumor cells. These compounds can be administered to a patient for the treatment or prevention of MUC1-positive cancers. Compounds that inhibit MUC1 cancers can act by a variety of mechanisms described herein. In certain embodiments, the compounds bind to the MGFR portion of the receptor and inhibit its growth factor activity. Other compounds described herein inhibit the MUC1 receptor indirectly by inhibiting enzymes that cleave the receptor. In certain embodiments, compounds are described that bind to the MUC1 receptor and also have a metal chelate functionality, which inactivates metal-dependent proteins and thus inhibits MUC1 cleavage. Methods are also described for disabling the tumorigenic activity of the MUC1 receptor via the use of agents that inhibit the expression of MUC1, e.g. through the use of anti-sense DNA or inhibitory RNAs called RNAi, inhibit the proteolysis of MUC1, inhibit post-translational modifications of MUC1 and inhibit natural ligands of MUC1 either directly or indirectly. Other compounds described herein act on intracellular proteins and are delivered to the intracellular region by MUC1 as it shuttles from the cell surface to the interior of the cell. Compounds are described that inhibit cell proliferation by inhibiting proteins from the kinesin family and in particular embodiments inhibit these proteins by abstracting a cationic metal from them. Compounds having the ability to chelate a cationic metal are also described, wherein they inhibit the activity of enzymes and receptors by removing or scavenging physiologically relevant cationic metals.

It has been reported that the MUC1 receptor can be cleaved, releasing a portion of the receptor and leaving a portion attached to the cell surface. Elevated levels of the shed portion of MUC1 can be detected in the blood of Stage II and III breast cancer patients. These levels increase as cancer progresses, which implies that receptor cleavage and cancer progression are related. The inventors have reported in a previous application (see, e.g. U.S. Patent Application Publication Nos. 2003/0036199 A1 and 2003/0130293 A1, incorporated by reference herein in their entirety) that cleavage products that remain attached to the cell surface are preferentially produced in tumor cells. These low molecular weight species are the result of cleavage, which occurs at at least two different sites and run with an apparent molecular weight of approximately 20 kD after deglycosylation. The portions that remain attached to the cell surface may consist essentially of PSMGFR (SEQ ID NO: 13) or TSESMGFR (SEQ ID NO: 28). The inventors previously disclosed that the PSMGFR (Table 1, SEQ. ID No. 36) portion of the MUC1 receptor can be the necessary and sufficient extracellular portion of the receptor that mediates cell growth and enables anchorage-independent cell growth, which is a characteristic of tumor cells. Therefore, agents that bind to the PSMGFR or to ESPSMGFR may be potential therapeutic compounds for the treatment or prevention of MUC1-positive cancers. Since the MUC1 cleavage product is linked to the growth factor activity of MUC1 and tumorigenesis, even more potent therapeutics may be compounds that can bind to the MUC1 receptor and block its interactions with activating ligands and also possess a functionality that prevents cleavage of the receptor or neighboring MUC1 receptors. Also effective may be compounds that can bind the MUC1 receptor and have a metal chelate functionality to inhibit MUC1 cleavage by a metalloprotease. In certain embodiments, preferred are compounds from the quinazolinone family and from the benzthiophene equivalent family, such as Compound No. 84 that also possess a metal chelate function.

Until now, the enzyme(s) that cleaves MUC1 was unknown. Because the amount of shed MUC1 receptor found in the blood increases with the progression of the disease and because the inventors previously disclosed that the cleavage product PSMGFR is the sufficient portion of the receptor that mediates the growth factor activity of the receptor, the present inventors have determined, in the context of certain aspects of the invention, that agents that inhibit the enzymes that are able to cleave MUC1 are therapeutic targets for the treatment and prevention of MUC1-positive cancers. It has been reported that some of the MMPs (matrix metalloproteases) are over-expressed in cancers and certain phosphatase inhibitors that induce expression of some MMPs have been shown to both increase cell proliferation and increase the shedding of MUC1. However there are numerous MMPs and ADAMs, which cleave a variety of substrates some of which may be related to cancer and some not. Therefore, in the context of the design and/or discovery of therapeutics, one aspect of the invention involves methods to determine which metalloproteases cleave what substrates and to determine the biological outcome of the inhibition of each.

Agents that inhibit MUC1 cleavage may be used to treat patients with MUC1-positive cancers. The inventors have determined in the context of certain aspects of the present invention, that agents that inhibit Furin also inhibit the cleavage of MUC1, and thus can be used to treat or prevent MUC1-positive cancers. Furin is an enzyme that cleaves a pro-domain from other enzymes and this cleavage is required for them to be in their active state. Furin processes the six MT-MMPs from the pro-enzyme to their active state. TIMPs (tissue inhibitors of metalloproteases) are small proteins that bind to and inhibit the cleavage activity of several MMPs. The present inventors have determined in the context of certain aspects of the present invention, that TIMP 2 and TIMP 3 inhibited the cleavage of MUC1. MT1-MMP, which is also known as MMP-14, is processed to its active state by Furin. Further, TIMP 2 and TIMP 3 are known to inhibit MT1-MMP. It is herein disclosed that MT1-MMP is able to cleave MUC1 to produce cleavage products that function as growth factor receptors and consist essentially of the PSMGFR and/or the ESPSMGFR. Thus, inhibitors of MT1-MMP may be therapeutics for the treatment of MUC1-positive cancers. The present specification also discloses, in the context of certain aspects of the invention, that TIMPS 2 and 3 inhibit the cleavage of MUC1 and thus may be therapeutics for the treatment of MUC1-positive cancers. Since these cleavage enzymes are dependent on the presence of Zn for activity, agents that chelate Zn are preferred, in certain embodiments, as therapeutic agents for the inhibition of MUC1-positive cancers. In certain embodiments, therapeutic agents for the treatment of MUC1 cancers are agents that can bind to the MGFR region of the MUC1 receptor and also have a metal chelate functionality wherein the agent can chelate Zn or Mg.

### MGFR Binding Compound That Inhibits Cleavage of MUC1

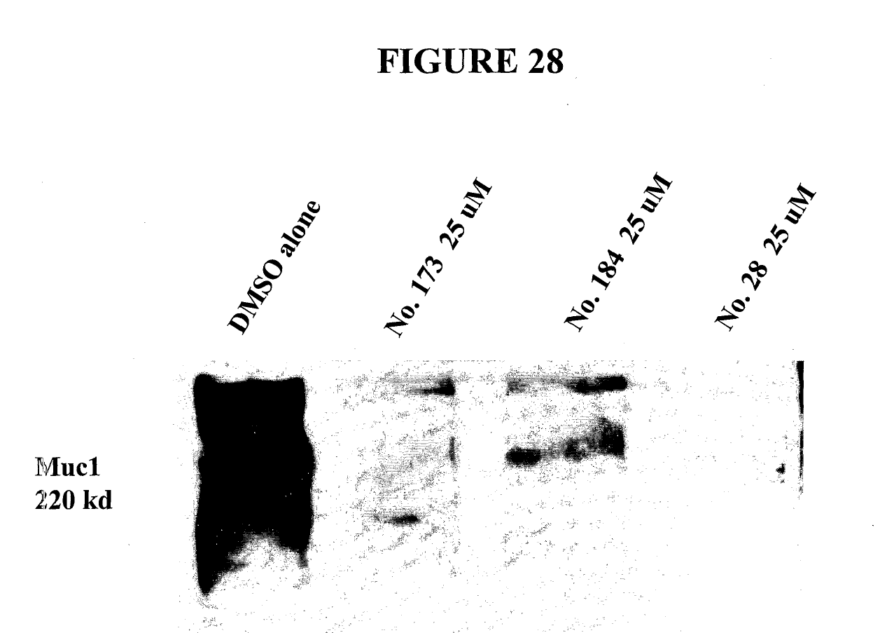

The compound shown in Table 4, Compound No. 173, has a metal chelate moiety that is able to chelate Mg, Zn and Ni. Compounds Nos. 184 and 28 also have metal chelate functionality and are able to chelate the metals Mg and Zn. Therefore, these compounds are able to inhibit the activities of metal-dependent proteins and particularly metal-dependent cleavage enzymes. It has also been discovered in the context of certain embodiments of the present invention, that Compounds Nos. 173, 184 and 28 inhibit the cleavage of the MUC1 receptor on growing cells, (See Example 5 and FIG. 28). These compounds also have the ability to bind to the MGFR portion of MUC1 and thus can be especially potent inhibitors of cell proliferation because they bind to and block the growth factor receptor portion of MUC1 and also localize the metal chelate functionality to a position near the site where a metal-dependent protease would cleave MUC1. Compounds Nos. 173, 184 and 28 are dual function compounds that can inhibit cleavage of MUC1 to prevent the production of the growth factor receptor form of the receptor and also block the interaction of the MGFR with its activating ligands. Thus, Compounds Nos. 173, 184 and 28, may be excellent therapeutics for the treatment of MUC1-positive cancers and other cell proliferative disorders that involve the MUC1 receptor. Virtually any composition of the invention or agent that binds to the PSMGFR region of MUC1 and possesses a metal chelate functionality would be excellent therapeutics for MUC1-positive cancers.

An agent that is able to bind to the PSMGFR region of MUC1 that does not have a metal chelate moiety can be derivatized with any metal chelate moiety, which are familiar to those skilled in the art. More broadly, Compounds Nos. 173, 184, and 28 may be therapeutics for conditions in which the inhibition of a metal-dependent protein has beneficial effects. Conversely, for some indications, the presence of the metal chelate function may introduce toxic effects in the recipient. In these cases, compounds such as Compound Nos. 173, 184 and 28 can be modified such that the moiety is no longer capable of chelating a metal. Comparison of Compound Nos. 173 and 186 demonstrates one example of how this can be accomplished.

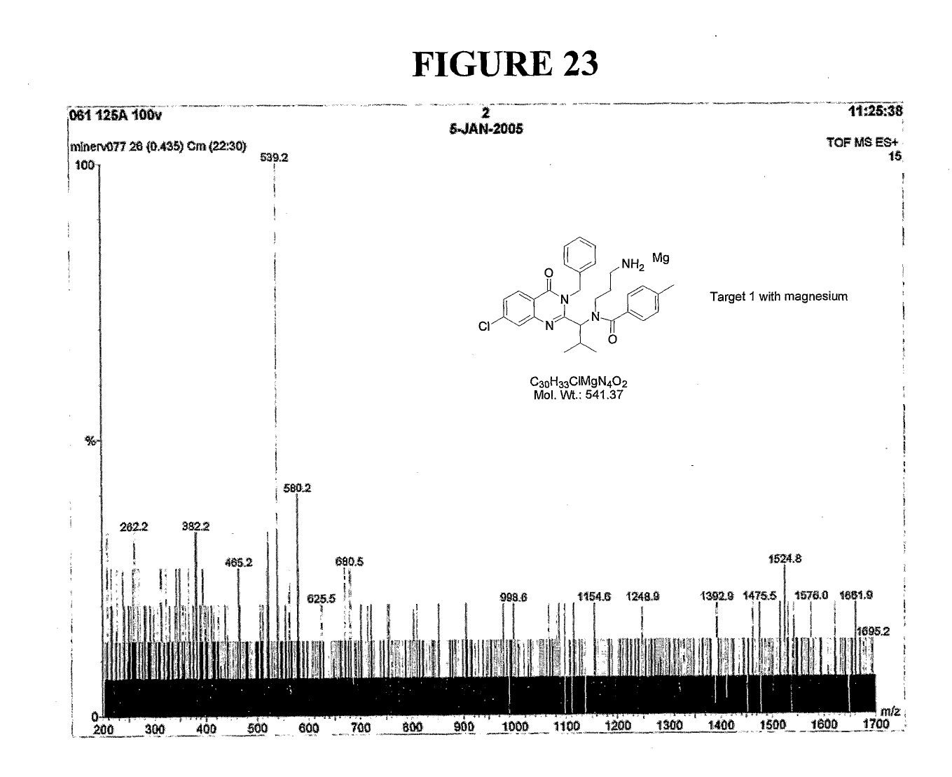

The conversion of Compound No. 173 to a novel analogue not capable of chelating a metal was achieved in a straight-forward fashion. The primary amine of Compound No. 173 was replaced by a methyl group in a manner well understood in the practice/art of medicinal chemistry. The resulting novel compound displayed no tendency to chelate a metal under conditions previously established for the observation of metal chelation by Compound No. 173. Mass Spectroscopy shows that Compound 173 chelates Mg but 186 does not, See Examples 7 and 8 and FIGS. 23, 24 and 25. Both compounds are still able to bind to the PSMGFR peptide and both still inhibit the growth of MUC1-positive tumor cells. Without being bound by theory, Compounds Nos. 173, 184 and 28 and similar compounds, e.g. those having a metal chelate functionality and in certain embodiments a metal chelate functionality and a motif that increases the concentration of the compound at the target site, can potentially be used to treat essentially any condition mediated by metal-dependent proteins. For example, these types of compounds can be, or can potentially be, used to treat proliferative diseases, including cancers that are mediated by Hedgehog proteins and particularly Sonic Hedgehog proteins by abstracting a metal from the hedgehog protein itself or by abstracting a Zn metal from Cos 2 (Costal2), which is a kinesin-like protein that is operative in the intracellular hedgehog signaling pathway. Compounds Nos. 173, 184 and 28 and similar agents that can chelate Mg can also be therapeutic agents for the treatment of cancers because they abstract a Mg atom from KSP (kinesin spindle protein) and inhibit its processing of ATP. Compounds with a metal chelate function can also be used to treat diseases that are mediated by proteins that span the membrane multiple times and involve a metal, the removal of which causes that loop to be internalized by the cell to shut down signaling. An example of this type of receptor is the melatonin hormone receptor which complexes Zn on one of the loops. Removal of the Zn atom causes that loop to be internalized and affects the regulation of the signal.

It is known that some cell surface receptors shuttle back and forth from the cell surface to the interior of the cell. It has been previously reported that MUC1 receptor shuttles from the cell surface to the cell interior. It has been proposed that the MUC1 receptor is glycosylated during this shuttling process. The present invention, in certain embodiments, provides a method whereby the shuttling mechanism of the MUC1 receptor is exploited to deliver compounds to the cell interior where they act on intracellular targets. For example, Compounds Nos. 173, 184 and 28 have a functionality that allows them to bind to the MGFR portion of the MUC1 receptor and a functionality that chelates a metal. According to an embodiment of the inventive method, the shuttling mechanism of the MUC1 receptor can be exploited to cause the concentration of Compounds Nos. 173, 184 and 28 within MUC1-positive cells to be much higher than in other cells, thereby concentrating the agent at the site of therapeutic interest. Within the cell, these compounds may abstract Mg or Zn from proteins that are required for cell division, e.g. KSP and Costal2 (Cos2). Therapeutic compounds that have a first functionality that binds to a cell surface receptor that shuttles back and forth across the cell membrane and a second functionality that acts on intracellular targets may be especially effective therapeutics. Preferred compounds, for certain embodiments, are those that have a first functionality that binds to a portion of a receptor that can be internalized and a second functionality that chelates a metal. Preferred, in certain embodiments, are compounds that have a first functionality that binds to the PSMGFR (SEQ. No 36) and/or the TSESMGFR (SEQ. No 66) and a second functionality that chelates Mg, Zn and/or Ca. In certain embodiments, the portion that binds to the PSMGFR and/or TSESMGFR is from the quinazolinone family and the metal chelate functionality can chelate magnesium and/or zinc, to inactivate KSP and/or Cos2.

It is beneficial to determine whether or not cancers present an aberrant form of the MUC1 receptor because its aberrant expression has been observed in a large percentage of cancers. Anti-proliferative compounds that act on the MUC1 receptor and/or on the MUC1 signaling pathway are described herein. Therapeutics that act through a MUC1 mechanism would typically not be effective in MUC1-negative cancers, i.e. those cancers characterized by cells that may present the MUC1 receptor in its normal form. Therapeutic agents that act on MUC1 and/or a MUC1-related pathway could be especially effective against MUC1-positive cancers, i.e. those cancers characterized by cells that aberrantly express the MUC1 receptor. Therefore, to provide more effective patient care it is, as taught in the context of certain embodiments of the invention, desirable that cancers be characterized to determine whether or not they aberrantly express the MUC1 receptor, wherein aberrant expression is taken to mean that the cells present a proteolyzed MUC1 that consists essentially of PSMGFR or ESPSMGFR, and/or the MUC1 receptors are unclustered and uniformly distributed on the cell surface and/or MUC1 is overexpressed. It is noted that MUC1 clustering at the apical border of cells forming a duct is a normal condition even when the predominant form of the receptor at the luminal surface of the duct consists essentially of PSMGFR. Specimens can be analyzed, according to certain embodiments of the invention, for the presence of aberrant MUC1 expression to determine an appropriate course of treatment, for example administration of one or more compositions of the invention, and/or to determine a patient population susceptible to benefit from treatment with a composition disclosed herein (e.g. a population having a cancer characterized by aberrant expression of MUC1).

A variety of standard and non-standard methods can be employed to determine whether a specimen contains the MUC1 receptor or an aberrant form of the MUC1 receptor. In the most simple case, a biopsy specimen is stained with an antibody, such as VU4H5 (Santa Cruz) or CA15.3 (Roche Diagnostic) or CA 27.29 (Biomira), which recognize the tandem repeat units of the MUC1 receptor. The probe antibody is either optically active or is reacted with a secondary antibody that is or can be made optically active or otherwise detectable. Thus, visual inspection of the stained specimen reveals whether or not the tumor was MUC1 positive or negative and suitable therapies can be determined.

Those skilled in the art will appreciate that there are several techniques suitable for determining the presence of the MUC1 receptor in a sample. One such method involves analyzing nucleic acids, which may include DNA or RNA, to determine if they contain MUC1 sequences. Antibodies and other ligands that specifically recognize portions of the MUC1 receptor are also used to probe for the presence of the receptor in, for example, sandwich assays such as ELISAs and western blots assays to determine the presence of the MUC1 receptor or portions thereof in the specimen. Chemiluminescent technology can also be used to determine binding of a recognition agent to the MUC1 receptor. An example of an antibody that has been used to probe for the presence of the MUC1 receptor in samples is VU4H5 and CA 15.3. These antibodies and others recognize portions of the MUC1 receptor that are distal to the cell surface and which contain tandem repeat units. These and other cognate entities can be used in any assay that indicates the presence of the MUC1 receptor or portions thereof.

However, because the MUC1 receptor is subject to proteolysis, use of diagnostic antibodies recognizing certain portions of the receptor may be advantageous over use of those recognizing others. The present application discloses that proteolyzed MUC1 is preferentially produced in tumor cells, the extracellular portion of the cleavage products of which may consist essentially of PSMGFR and TSESMGFR. Additionally, the inventors have discovered that the PSMGFR is a sufficient portion of the MUC1 receptor that mediates cell growth and enables anchorage-independent cell growth, which is characteristic of tumor cells.

Antibodies against the tandem repeat units of the MUC1 receptor are commercially available and can be used as diagnostic reagents. However, these antibodies are of limited use to detect MUC1 in samples derived from cells or tissue specimens, especially when the samples are derived from a tumor, for a number of reasons. On tumor cells, many of the receptors are typically cleaved, so that the tandem repeat units are no longer attached to the cell surface. This means that probing a patient specimen with this antibody will produce conflicting results. In an extreme case, wherein all of the MUC1 receptors have been cleaved to the MGFR form, which functions as a growth factor receptor, the diagnostic assay would read MUC1-negative reflecting the lack of tandem repeats but missing the fact that the growth factor form was fully expressed. If a patient's specimen has a low level of reactivity with the antibody against the terminal repeats, it may be because there is a low level of MUC1 expressed on the cell surface or there is a high level of receptor cleavage and by extension a high level of tumor cell proliferation. A further complication of using antibodies that bind to the tandem repeat units is that the number of repeats varies from person to person and this variation has not been linked to cancer. Therefore, a high level of antibody reactivity with a patient specimen may mean that there is an abundance of MUC1 receptor expressed on the surface of the cells or may mean that the patient expresses MUC1 with a greater than average number of repeat units attached to each receptor.

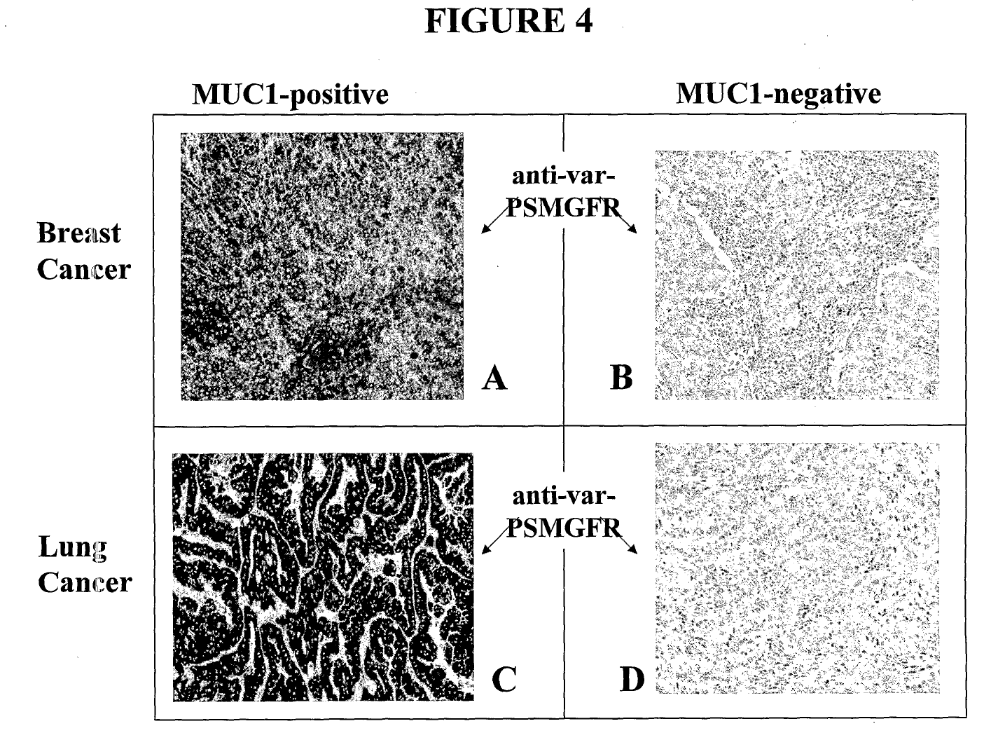

It is herein disclosed that an antibody that was raised against the peptide whose sequence corresponds to PSMGFR, referred to herein as anti-PSMGFR or anti-var-PSMGFR can be used to diagnose MUC1-positive cancers, including but not limited to breast, lung, colorectal, pancreatic, prostate and ovarian cancers. Anti-var-PSMGFR was used to examine human tissue specimens derived from breast, lung, colon, pancreas, ovary, and prostate. The tissue specimens had been characterized by a pathologist. The tumors came from cancerous, benign normal, and benign displastic conditions. MUC1-positive cancer specimens from breast, colon, and lung were clearly identifiable as cancers strictly from the degree of anti-var-PSMGFR. Antibodies against the tandem repeat units of the receptor were inferior in performance and in most cancerous tissues, often did not stain. See Example 3 and FIGS. 4-10. Antibodies against the cytoplasmic tail of MUC1 are commercially available. These antibodies could be used to probe patient specimens that have been processed such that they are amenable to analysis using SDS-PAGE or western blots. Using this type of assay, the antibody against the cytoplasmic tail may reveal whether or not the MUC1 receptor was expressed in the specimen and the approximate molecular weights of the expressed MUC1 species, but would not provide any information about receptor clustering or patterning on the cell surface. Additionally, these assays are lengthy and labor intensive.

Antibodies that recognize portions of the UR may be useful for some diagnostic purposes, but since this portion of the MUC1 receptor is typically shed from the cell surface following MUC1 cleavage in tumor cells, its use as a diagnostic reagent for probing tissues and cells is limited. An antibody that recognizes this portion of the receptor may be useful for determining the ratio of cleaved to uncleaved receptor.

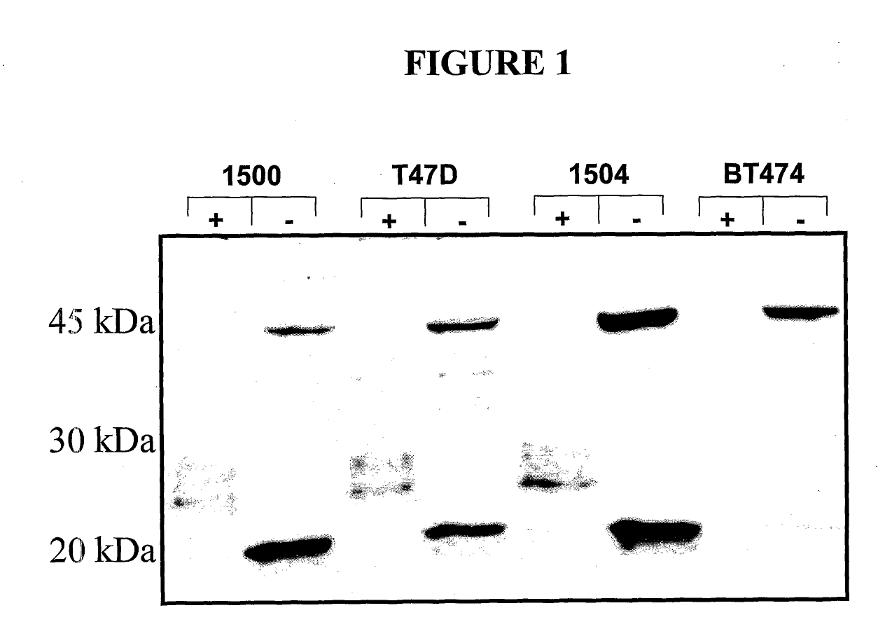

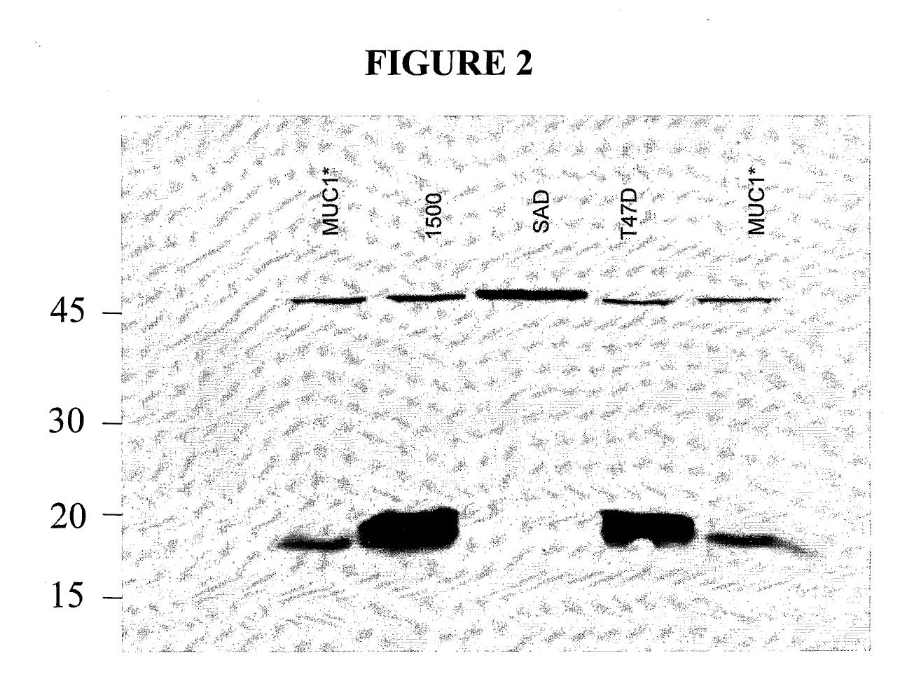

Antibodies that bind to the MGFR portion of the MUC1 receptor are preferred, in certain embodiments, as diagnostic reagent for probing cells and tissue specimens. In certain embodiments, the MUC1 diagnostic antibody recognizes a portion or portions of the ESPSMGFR. In certain embodiments, the MUC1 diagnostic antibody recognizes a portion or portions of the PSMGFR. The inventors have determined that an antibody raised to the nat-PSMGFR (See FIGS. 1 and 2) specifically binds to the MUC1 receptor, whether it is the full-length receptor or the proteolyzed fragment, which is produced by tumor cells because on tumor cells, the receptors are not clustered and the PSMGFR epitope is available to antibody binding.

The inventors have determined that this antibody specifically detects essentially all MUC1 species when used in western blot analysis, visualization of the MUC1 receptor on the surface of cells either free in solution or in a tissue specimen, and the like. The inventors have also produced an antibody against the var-PSMGFR and have demonstrated that this antibody is more specific than the antibody raised against the native sequence for probing cells, tissue specimens, for use in western blots, and in other analytical methods where the aim is to detect all the MUC1 expressed on cell surfaces. Antibodies raised against the truncated var-PSMGFR (SEQ ID NO: 6) may also be effective for probing cells, tissue specimens, for use in western blots, and in other analytical methods where the aim is to detect all the MUC1 expressed on cell surfaces.

The inventors have demonstrated that antibodies directed against a portion or portions of the IBR can be useful for determining a subject's susceptibility to cancer or that a subject had an acute cancer developing. Detecting the presence of a portion of the IBR that has been detached from the cell implies that the portion of the MUC1 receptor that remains on the cell surface is functioning as a growth factor receptor and is an indicator of the presence of or a predisposition to cancer. Detecting the presence of the IBR region, detached from the cell surface, in a subject's blood, serum, urine, milk, breast secretions or other bodily sample is evidence that the tumor-related cleavage products PSMGFR or ESMGFR, are left attached to the cell surface and are functioning as growth factor receptors to increase tumor development and cancer progression. Antibodies against PSMGFR, IBR, UR and the tandem repeat units used singly or in combination can provide information regarding the aggressiveness of the patient's cancer and can be useful for probing samples derived from bodily fluids as well as samples involving cells and tissue. A determination of cancer aggressiveness may be made by using combinations of antibodies and thereby determining the ratio of cleaved receptor to uncleaved receptor. That is to say that the absolute level of MUC1 receptor that each patient expresses may vary depending upon the individual or cancer grade, however, the higher the percentage of cleaved MUC1 receptor compared to uncleaved, the greater the cancer potential and tumor aggressiveness. Combinations of antibodies that recognize different portions of the MUC1 receptor may be used to determine which portions and relative amounts of the MUC1 receptor are present on the cell surface or in the circulation as a method to diagnose, characterize, assess metastatic potential, design therapeutic protocols and track the patient's response to those therapies.

Agents that have a signaling capability, e.g. antibodies, cognate proteins, or small molecules, that bind to a portion or portions of the PSMGFR or ESPSMGFR are useful, in certain embodiments, as diagnostic agents to detect whether or not a cell, tissue specimen or other sample presents a MUC1 species that promotes cell proliferation. Other agents, e.g. antibodies, that bind to the PSMGFR and that have a signaling capability are preferred, in certain embodiments, as diagnostic agents to detect whether or not a cell, tissue specimen or other sample presents a MUC1 species that promotes cell proliferation. Antibodies that bind to the PSMGFR portion are preferred, in certain embodiments, as diagnostic tools for determining whether a cancer is MUC1-positive or negative because these antibodies are capable of recognizing MUC1 in the cleaved or uncleaved state, however, they do not stain healthy tissue specimens even when cells within those specimens are known to express the MUC1 receptor. Preferrential staining of cancer tissues using antibodies against PSMGFR is due to the accessibility of the cognate epitope and could also be due in part to overexpression of the MUC1 receptor in tumor tissue. The use of antibodies that recognize portions of the IBR used in combination with an antibody that recognizes the UR or the tandem repeats are, in certain embodiments, useful for diagnosing MUC1-positive cancers wherein the sample is derived from a patient's bodily fluid. High levels of IBR that has been released from the cell surface is indicative of cancer and particularly high levels are indicative of an aggressive cancer.

Agents that have a signaling capability, e.g. antibodies, cognate proteins, or small molecules, that bind to a portion or portions of the MGFR, such as to the PSMGFR sequence, are useful, in certain embodiments, as diagnostic agents to detect tumors or cancers in whole body diagnostic applications, such as MI or PET scans. Such agents are also useful in some embodiments as agents to render cancerous tissues visible for surgical removal. Compositions of the invention are useful as such since they can readily be modified with signaling entities for any one of a number of desired applications.

Small molecules are especially preferred for these applications. Methods to modify compositions of the invention with signaling entities are known to those skilled in the art. Signaling entities with which to modify these compositions will vary depending upon the application. For example, the most common nuclei detected with MRI are H, F, Si, P. Contrast agents in MRI are typically transition metals with some targeting organic attached (chelation of the metal with an organic molecule). These agents alter the relaxation times of the detected nuclei of interest and allow imaging at localized sites. PET (positron emission topography) scanning which uses ¹⁸F as the contrast agent after incorporation into an organic molecule that is localized in the body. These and several other signaling entities can be readily attached to agents that bind to the PSMGFR to produce imaging agents that are targeted to the tumorigenic form of MUC1.

Patient specimens that are analyzed to determine the presence of, or the cancerous potential of, the MUC1 receptor, according to certain embodiments of the invention, may include tumor specimens, tissue specimens, needle biopsy material, cells extracted from a blood sample, the shed portion of the MUC1 receptor in a blood sample or other bodily fluids including breast milk, or MUC1-associated factors, such as ligands and modifying ligands in the blood and in other specimens.

In certain embodiments, a tumor is excised from a patient and analyzed to determine whether or not it is cancerous and whether or not it aberrantly expresses MUC1 by treating the specimen with an antibody(s) directed against the MUC1 receptor. MUC1 is typically expressed in a wide range of epithelial cells but typically is only detectable at the apical border of cells in healthy tissues. Visual inspection is made to determine whether or not the expressed MUC1 is clustered or is expressed over the entire cell surface, which is characteristic of MUC1-associated cancers. This type of MUC1 expression is aberrant and such cancers would be termed MUC1-positive cancers. The expression of certain molecules in tissue specimens can be detected by a variety of methods that are known to those skilled in the art. Several immunohistochemistry (IHC) techniques, staining reagents and detectable secondary antibodies are typically used to render specific molecules visible in tissue biopsy and needle biopsy specimens.

The levels of PSMGFR that are expressed on cells is compared to established levels that would normally be present on healthy MUC1-positive cells. High levels of MUC1 species that interact with anti-PSMGFR are an indication of cancer and its aggressive potential. In certain embodiments the specimen is probed with anti-PSMGFR and an antibody that recognizes the unique region, and the ratio of PSMGFR reactivity to anti-unique region reactivity is calculated. A high ratio of PSMGFR unique region, i.e. more cleaved MUC1 is present than uncleaved MUC1 indicates an aggressive cancer and thus this measurement is used for prognosis and to design appropriate therapies. If the specimen is determined to be cancerous and expresses MUC1, the condition is treated with a compound that directly binds to the PSMGFR portion of the receptor, such as compounds from Tables 2, 3, 4, and 5. Especially preferred are Compounds Nos. 173, 184, 28, 185, 118, 125, 182, 188, 107 and 109.

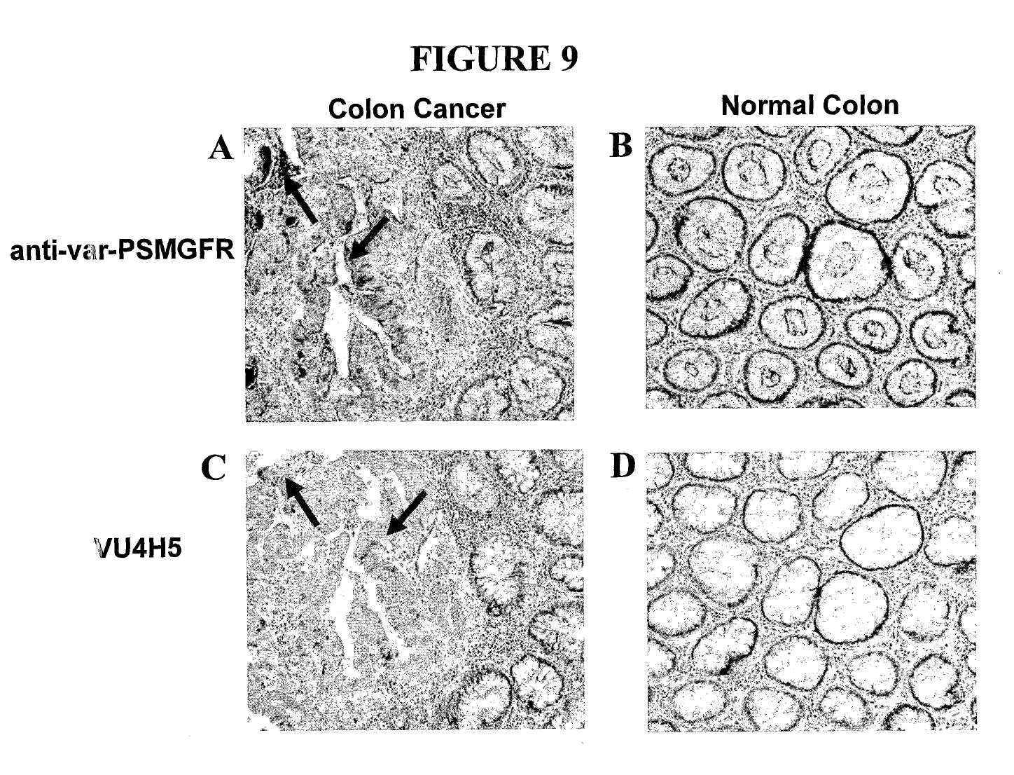

When non-cancerous tissue specimens were probed with a commercially available antibody, VU4H5 (Santa Cruz) that binds to the tandem repeat units, cytoplasmic staining was sometimes observed within cells that lined normal ducts. Other normal ducts did not stain positive for the antibody that recognizes the tandem repeat units. However, when some cancerous tissue specimens were treated with antibodies against the tandem repeats, large cancerous regions that did not involve ducts stained positive with the antibody. Staining with antibodies directed against the tandem repeat units was diffuse and cytoplasmic. A limitation for the use of antibodies against the tandem repeat units for characterizing cancers is that some of the most cancerous regions of the specimens did not stain positive with these antibodies. An extrapolation of this observation is that late stage cancers that are driven by aberrant MUC1 expression would stain negative with these antibodies against portions of the receptor that are shed. These antibodies are blind to the portion of the receptor that functions as a growth factor receptor to drive tumor growth. For an example of cancerous tissue that stained positive with an antibody against the MGFR of MUC1 but negative with an antibody against the distal tandem repeat units, (See Example 3 and FIG. 9).

In an especially preferred embodiment, tumor tissue specimens were characterized as to whether or not they were MUC1-positive or negative cancers by treating the specimens with an antibody raised against the var-PSMGFR (SEQ. ID No. 7), which is referred to herein as anti-PSMGFR CB, (See Example 3, FIGS. 4-10). The inventors herein disclose that the MUC1 species that is present on cells of cancerous tissues is comprised essentially of PSMGFR and is devoid of the tandem repeat units, i.e., essentially all of the MUC1 receptors on the surface of cancerous cells have been cleaved and shed to release a portion of the receptor that at least includes the tandem repeats and leaves a portion of the receptor attached to the cell surface that contains the PSMGFR. Anti-PSMGFR intensely stained all the cancerous regions of the specimens. Staining was membrane and showed uniform distribution of the receptor over the cell surface. In some instances the staining resembled a chicken wire pattern. Ducts in cancerous regions were intensely stained and staining was not limited to the apical border, but was spread over the entire cell surface. 4-5 layers of cells surrounding the duct stained positive and also displayed uniform membrane staining. This is in contrast to the staining of normal non-cancerous ducts with anti-PSMGFR wherein a single layer of cells that form the duct, stained positive only at the apical border. Each tissue specimen analyzed had regions of the specimen that was made up primarily of healthy, normal cells. These healthy cells did not stain positive with the anti-PSMGFR antibody. The inventors herein disclose that cancerous tissue specimens are readily differentiated from non-cancerous specimens or from MUC1-negative tumor specimens by simple visual observation of anti-PSMGFR staining of cells in regions away from the luminal edge of ducts, and/or by observing anti-PSMGFR staining that is uniformly distributed over the entire cell surface and/or by detecting bulk staining of areas of the tissue specimen with anti-PSMGFR.

The use of anti-PSMGFR is superior to the use of antibodies that recognize the tandem repeats for characterizing specimens as to whether they are MUC1-positive or negative cancers. Because nearly all the MUC1 receptors on tumor cells have been cleaved to shed and release the tandem repeat domain, staining is limited to diffuse cytoplasmic staining. In very cancerous regions of tissue, no MUC1 staining is detectable using these antibodies. Additionally, ducts that appear normal in “normal” areas of cancerous specimens did not stain positive with antibodies against the tandem repeats. These effects would give rise to false negative diagnoses if specimens were probed with antibodies against the portions of the MUC1 receptor such as VU4H5 that are shed from the cell surface. In addition, necrotic sections of MUC1-negative cancers stain positive with antibodies against the tandem repeats even though those cancers were MUC1-negative. This gives rise to false positive diagnoses. In contrast, because the PSMGFR domain of the MUC1 receptor is the portion that acts as a growth factor receptor to mediate the growth of tumor cells, staining with anti-PSMGFR is a direct readout of the amount of MUC1 in the specimen that is acting as a growth factor receptor for the cells involved. The degree of staining is directly proportional to the tumorigenic potential of the sample under analysis. The level of anti-PSMGFR staining can be used to diagnose cancer, to diagnose MUC1-positive cancers, and may be used as an indicator of tumor grade and metastatic potential.

Many tumors, especially breast tumors, are characterized as benign, i.e. not cancerous, but contain a number of displastic cells. For many benign conditions and tumors, it has not been possible to predict which are likely to develop into cancers. If a method were available that would predict the potential for a benign but displastic tumor to become cancerous, early treatment protocols could be administered which would result in higher cure and survival rates. The inventors herein disclose that antibodies that bind to the MGFR portion of the MUC1 receptor can be used to identify “benign” conditions that are likely to develop into cancers. Anti-PSMGFR stains a ring inside displastic cells of cancerous specimens but not displastic cells in other non-cancerous specimens, including specimens from conditions that are known not to evolve into cancer but possess a high degree of displasia. Therefore, anti-PSMGFR is useful for staining displastic cells, but not cancerous tumors, to predict their potential for developing into cancers. Those that would progress to cancer would stain an internal ring within the displastic cells of a needle biopsy, or tissue biopsy, or retrieved circulating cells.

The present invention also involves, in one aspect, methods for treating patients susceptible to or exhibiting symptoms of a tumorigenic condition or a condition where healthy receptor clustering has been disrupted.

The present invention also provides for the treatment of patients for a condition different from cancer, including conditions that can be unrelated to cancer, in some embodiments of the present invention. If a composition of the invention is known for treatment of a different condition, the present invention also involves use of that composition for treatment of cancer where indicated. The present invention, in certain embodiments, also includes treatments where the dosage, delivery technique or vehicle, combination with other pharmaceutical compositions or lack of combination with other pharmaceutical compositions, rate of administration, timing of administration, or other factor differs from the use of the composition for treatment of the condition different from cancer.

In another set of embodiments, the invention is directed to treating a patient population never before treated with drugs useful according to certain methods of the invention, including patients who are not suffering from or indicating susceptibility to abnormal cell proliferation, cancers or tumors, particularly MUC1-associated cancers. In other words, the treatment preferably is directed to patient populations that otherwise are free of symptoms that call for treatment with any of the drugs useful according to the invention.

PCT/US01/12484 (WO 01/78709), filed Apr. 12, 2001 by Bamdad et al., PCT/US00/01997 (WO 00/43791), filed Jan. 25, 2000 by Bamdad et al., and PCT/US00/01504 (WO 00/34783), filed Jan. 21, 2000 by Bamdad, et al., are incorporated by reference herein in their entirety. Also incorporated by reference herein are the following: PCT/US01/44782 (WO 02/056022), filed Nov. 27, 2001, by Bamdad, et al.

The present invention, in certain embodiments, involves compositions related to cancers and methods of treatment of cancers characterized by the aberrant expression of a class of cell surface receptors characterized by interchain binding regions. One such set of cancers are those cancers characterized by the aberrant expression of MUC1. Much of the description of the invention herein involves cells that aberrantly express MUC1. It is to be understood that in these instances the description is to be considered exemplary, and that the principles of the invention apply to other cell surface receptors that function by a similar mechanism. With the disclosure herein, those of ordinary skill in the art will readily be able to identify other cell surface receptors that function by this or a similar mechanism, and to apply the invention to those cancers characterized by aberrant expression of those receptors. The invention is based on a novel mechanism involving aberrant expression of cell surface receptors, exemplified by MUC1, which was elucidated by the inventors.

One aspect of the invention is directed to a method for treating a subject diagnosed or at risk of cancer or tumor characterized by the aberrant expression of MUC1. The treatments of the present invention involve the use of compositions or “agents” as described herein. That is, one aspect of the invention involves a series of compositions or agents useful for treatment of cancer or tumor characterized by the aberrant expression of MUC1. These compositions may also be packaged in kits, optionally including instructions for use of the composition for the treatment of such conditions. These and other embodiments of the invention may also involve promotion of the treatment of cancer or tumor according to any of the techniques and compositions and combinations of compositions described herein.

One aspect of the invention provides a pharmaceutical preparation comprising a composition comprising any of compositions shown below (numbered 1-188), optionally with a pharmaceutically active carrier:

In one embodiment, the composition comprises homologs, analogs, derivatives, enantiomers and functionally equivalent compositions thereof of compositions 1-188. Another aspect of the present invention provides any of the above-mentioned compositions as being useful for the treatment of cancer and particularly MUC1-positive cancers. In particular, Compound Nos. 173, 184, 28, 185, 118, 125, 182, 188, 107 and 109 are preferred.

### Structure of Compounds

Certain embodiments of the present invention relate to compositions that are able to inhibit MUC1-related proliferative diseases, particularly cancers, involving inhibiting the portion of MUC1 that functions as a Growth Factor Receptor, cleavage of the full-length receptor to its tumorigenic form or interaction of the MUC1 receptor with its ligands, and methods for treating patients displaying symptoms of, or susceptible to MUC1-associated cancers by either inhibiting direct interactions or by inhibiting their expression. The subject matter of this application involves, in some cases, interrelated products, alternative solutions to a particular problem, and/or a plurality of different uses of a single system or article.

Several methods are disclosed herein of administering to a subject a composition for prevention or treatment of a particular condition. It is to be understood that in each such aspect of the invention, the invention specifically includes the composition for use in the treatment or prevention of that particular condition, as well as use of the composition for the manufacture of a medicament for the treatment or prevention of that particular condition. In some aspects of the invention, the invention also includes a pharmaceutically acceptable carrier.

The present invention includes methods of treatment of selected groups of patients. It is to be understood that all compositions described herein are useful or potentially useful for each described method.

Also included in certain embodiments of the present invention is a combinatorial approach in which structural features identified as characteristic of compositions effective for treatment at various disease stages are used as the basis for combinatorial synthesis of a wide variety of structural homologs, analogs, derivatives, enantiomers and functionally equivalent compositions thereof, for identification of a wide variety of compositions useful for treatment MUC1-associated cancers. Thus, in one embodiment, the invention involves providing any one or more of Compounds Nos. 1 to 188 as set forth in Tables 2 to 5, and performing a combinatorial synthesis resulting in a plurality of compositions. Then, one can perform an assay involving the plurality of the compositions to determine their effectiveness in cancer treatment, specifically, for example, treatment of cancers disclosed herein. Compounds Nos. 1-188 also can be altered using medicinal chemistry techniques.

Another aspect of the invention provides, in certain embodiments, a pharmaceutical preparation comprising a composition comprising any of the Compounds Nos. 1-188, and a pharmaceutically active carrier. In one embodiment, compounds may comprise homologs, analogs, derivatives, enantiomers and functionally equivalent compounds of Compounds Nos. 1 to 188. In all structures herein, atom locations, if unlabeled, are carbon with appropriate hydrogen(s). The invention also provides, in certain embodiments, a method involving promoting the prevention or treatment of MUC1-associated cancer via administration of any one or more of the compositions of the present invention and/or homologs, analogs, derivatives, enantiomers and functionally equivalent compositions thereof.

In another aspect, the invention provides a kit including any one or more of the compositions of the present invention and/or homologs, analogs, derivatives, enantiomers and functionally equivalent compositions thereof; and instructions for use of these compositions for treatment of cancer characterized by aberrant expression of MUC1.

In one aspect, the invention is defined, at least in part, by a method. In some embodiments of the invention, the method involves treating a human patient susceptible to or exhibiting symptoms of a cancer characterized by aberrant expression of MUC1 with any of the compositions disclosed herein. In one set of embodiments, the patient is susceptible to, but does not exhibit symptoms of, cancer characterized by aberrant expression of MUC1. In another set of embodiments, the patient exhibits symptoms of cancer characterized by aberrant expression of MUC1. In some embodiments of the method, the patient is not otherwise indicated for treatment for a cancer characterized by aberrant expression of a hedgehog protein.

In another aspect, the invention is directed to a method of making any of the embodiments described herein. In yet another aspect, the invention is directed to a method of using any of the embodiments described herein.

In one aspect, the invention involves a composition comprising compounds of a general structure and formula that can be routinely prepared by well-established methods. In one set of embodiments, the composition has a structure:

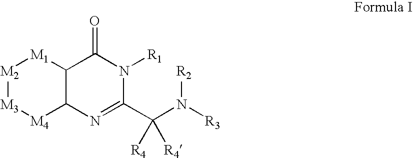

where M₁, M₂, M₃ and M₄ are each independently selected from the group consisting of carbon, nitrogen, sulfur, oxygen residues and/or an atomic null such that a fused bicyclic ring system providing valence satisfaction and chemical stability is achieved. Substitutions at positions M₁-M₄ on the above atoms may be hydrogen or halogen, such as fluorine, chlorine or bromine, or carbon-linked substituents such as methyl, ethyl, propyl, isopropyl and higher alkyl and aryl analogs, or nitrogen-linked substituents including amine, methylamine, dimethylamine or higher secondary or tertiary alkyl or aryl amines, or oxygen-, sulfur- and selenium-linked substituents including hydroxyl, sulfhydryl and selenylhydryl and alkyl and aryl ether analogs thereof, or silicon-, phosphorous- or boron-linked substituents including alkyl or aryl substitutions at these residues. In all cases, linking atoms and linking-atom substituents are as required for valence satisfaction and chemical stability. R₁ may be any atom or substituent other than halogen including hydrogen, methyl, ethyl, benzyl, aryl and substituted analogs thereof. R₂ and R₃ are each independently chosen to be hydrogen, or substituted oxygen-, carbon-, nitrogen- or sulfur-linked substituents such that valence satisfaction and chemical stability are achieved. R₂ and R₃ may be covalently linked to give a set of monocyclic aza-cycles. R₄ and R_{4′} may be independently hydrogen, carbon-, oxygen-, nitrogen- or sulfur-linked substituents including methyl, ethyl, isopropyl, higher alkyl and aryl substituents, hydroxyl, sulfhydryl, alkyl and aryl ethers and thioethers, amine, methyl amine, dimethyl amine and higher alkyl and aryl secondary and tertiary amines. In all cases, substitutions are chosen such that valence satisfaction and chemical stability are achieved. R₄ and R_{4′} may be covalently linked to give a set of cyclic compounds.

In another set of embodiments, the composition has a structure:

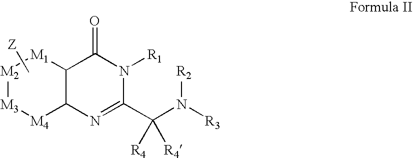

where M₁, M₂, M₃ and M₄ are each independently selected from the group consisting of carbon, nitrogen, sulfur, oxygen atoms and/or an atomic null such that a fused bicyclic ring system providing valence satisfaction and chemical stability is achieved. Single and multiple substitutions by the substituent(s) Z at positions M₁-M₄ on the above designated carbon, nitrogen and sulfur atoms may be hydrogen or halogen, such as fluorine, chlorine or bromine, or carbon-linked substituents such as methyl, ethyl, propyl, isopropyl and higher alkyl and aryl analogs, or nitrogen-linked substituents including amine, methylamine, dimethylamine or higher secondary or tertiary alkyl or aryl amines, or oxygen-, sulfur- and selenium-linked substituents including hydroxyl, sulfhydryl and selenylhydryl and alkyl and aryl ether analogs thereof, or silicon-, phosphorous- or boron-linked substituents including alkyl or aryl substitutions at these atoms. In all cases, linking atoms and linking-atom substituents are as required for valence satisfaction and chemical stability. R₁ may be any residue other than halogen, including hydrogen, methyl, ethyl, benzyl, aryl and substituted analogs thereof. R₂ and R₃ are each independently chosen to be hydrogen, or substituted oxygen-, carbon-, nitrogen- or sulfur-linked substituents such that valence satisfaction and chemical stability are achieved. R₂ and R₃ may be covalently linked to give a set of monocyclic aza-cycles. R₄ and R_{4′} may be independently hydrogen, carbon-, oxygen-, nitrogen- or sulfur-linked substituents including methyl, ethyl, isopropyl, higher alkyl and aryl substituents, hydroxyl, sulfhydryl, alkyl and aryl ethers and thioethers, amine, methyl amine, dimethyl amine and higher alkyl and aryl secondary and tertiary amines. In all cases, substitutions are chosen such that valence satisfaction and chemical stability are achieved. R₄ and R_{4′} may be covalently linked to give a set of cyclic compounds.

In another set of embodiments, the composition has a structure:

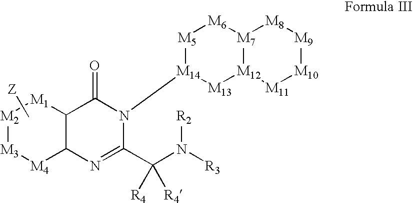

where M₁, M₂, M₃ and M₄ are each independently selected from the group consisting of carbon, nitrogen, sulfur, oxygen residue and/or an atomic null such that a fused bicyclic ring system providing valence satisfaction and chemical stability is achieved. Single and multiple substitutions by the substituent(s) Z at positions M₁-M₄ on the above designated carbon, nitrogen and sulfur atoms may be hydrogen or halogen, such as fluorine, chlorine or bromine, or carbon-linked substituents such as methyl, ethyl, propyl, isopropyl and higher alkyl and aryl analogs, or nitrogen-linked substituents including amine, methylamine, dimethylamine or higher secondary or tertiary alkyl or aryl amines, or oxygen-, sulfur- and selenium-linked substituents including hydroxyl, sulfhydryl and selenylhydryl and alkyl and aryl ether analogs thereof, or silicon-, phosphorous- or boron-linked substituents including alkyl or aryl substitutions at these atoms. In all cases, linking residues and linking-atom substituents are as required for valence satisfaction and chemical stability. M₅-M₁₄ may be carbon, nitrogen, oxygen or sulfur or any residue other than hydrogen or halogen and in certain embodiments, may be either a moiety where M₅, M₆, M₇, M₈, M₉, M₁₀, M₁₁, M₁₂, M₁₃ and M₁₄ are each independently selected from the group consisting of carbon, nitrogen, sulfur, oxygen atoms and/or an atomic null such that a monocyclic or bicyclic ring system providing valence satisfaction and chemical stability are achieved. R₂ and R₃ are each independently chosen to be hydrogen, or substituted oxygen-, carbon-, nitrogen- or sulfur-linked substituents such that valence satisfaction and chemical stability are achieved. R₂ and R₃ may be covalently linked to give a set of monocyclic aza-cycles. R₄ and R_{4′} may be independently hydrogen, carbon-, oxygen-, nitrogen- or sulfur-linked substituents including methyl, ethyl, isopropyl, higher alkyl and aryl substituents, hydroxyl, sulfhydryl, alkyl and aryl ethers and thioethers, amine, methyl amine, dimethyl amine and higher alkyl and aryl secondary and tertiary amines. In all cases, substitutions are chosen such that valence satisfaction and chemical stability are achieved. R₄ and R_{4′} may be covalently linked to give a set of cyclic compounds.

In another set of embodiments, the composition has a structure:

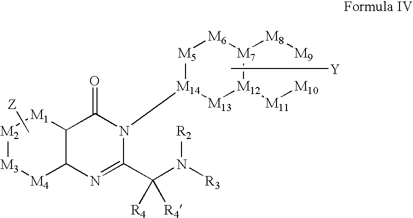

where M₁, M₂, M₃ and M₄ are each independently selected from the group consisting of carbon, nitrogen, sulfur, oxygen residue and/or an atomic null such that a fused bicyclic ring system providing valence satisfaction and chemical stability is achieved. Single and multiple substitutions by the substituent(s) Z at positions M₁-M₄ on the above designated carbon, nitrogen and sulfur atoms may be hydrogen or halogen, such as fluorine, chlorine or bromine, or carbon-linked substituents such as methyl, ethyl, propyl, isopropyl and higher alkyl and aryl analogs, or nitrogen-linked substituents including amine, methylamine, dimethylamine or higher secondary or tertiary alkyl or aryl amines, or oxygen-, sulfur- and selenium-linked substituents including hydroxyl, sulfhydryl and selenylhydryl and alkyl and aryl ether analogs thereof, or silicon-, phosphorous- or boron-linked substituents including alkyl or aryl substitutions at these atoms. In all cases, linking residues and linking-group substituents are as required for valence satisfaction and chemical stability. M₅-M₁₄ may be carbon, nitrogen, oxygen or sulfur or any atom other than hydrogen or halogen and, in certain embodiments, may be either a moiety where M₅, M₆, M₇, M₈, M₉, M₁₀, M₁₁, M₁₂, M₁₃ and M₁₄ are each independently selected from the group consisting of carbon, nitrogen, sulfur, oxygen atoms and/or an atomic null such that a monocyclic or bicyclic ring system providing valence satisfaction and chemical stability are achieved; furthermore, single and multiple substitutions by the substituent(s) Y at positions M₅-M₁₄ on the above designated carbon, nitrogen and sulfur atoms may be hydrogen or halogen, or substituted oxygen-, carbon-, nitrogen- or sulfur-linked substituents including methyl, ethyl, isopropyl, higher alkyl and aryl substituents, hydroxyl, sulfhydryl, alkyl and aryl ethers and thioethers, amine, methyl amine, dimethyl amine and higher alkyl and aryl secondary and tertiary amines such that valence satisfaction and chemical stability are achieved. R₂ and R₃ are each independently chosen to be hydrogen, oxygen-, carbon-, nitrogen- or sulfur-linked substituents including methyl, ethyl, isopropyl, higher alkyl and aryl substituents, hydroxyl, sulfhydryl, alkyl and aryl ethers and thioethers, amine, methyl amine, dimethyl amine and higher alkyl and aryl secondary and tertiary amines for valence satisfaction; R₂ and R₃ may be covalently linked to give a set of monocyclic aza-cycles. R₄ and R_{4′} may be independently hydrogen, carbon-, oxygen-, nitrogen- or sulfur-linked substituents including methyl, ethyl, isopropyl, higher alkyl and aryl substituents, hydroxyl, sulfhydryl, alkyl and aryl ethers and thioethers, amine, methyl amine, dimethyl amine and higher alkyl and aryl secondary and tertiary amines. In all cases, substitutions are chosen such that valence satisfaction and chemical stability are achieved. R₄ and R_{4′} may be covalently linked to give a set of cyclic compounds.

In another set of embodiments, the composition has a structure:

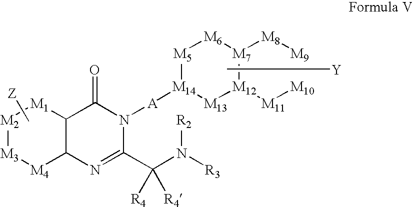

where M₁, M₂, M₃ and M₄ are each independently selected from the group consisting of carbon, nitrogen, sulfur, oxygen residue and/or an atomic null such that a fused bicyclic ring system providing valence satisfaction and chemical stability is achieved. Single and multiple substitutions by the substituent(s) Z at positions M₁-M₄ on the above designated carbon, nitrogen and sulfur atoms may be hydrogen or halogen, such as fluorine, chlorine or bromine, or carbon-linked substituents such as methyl, ethyl, propyl, isopropyl and higher alkyl and aryl analogs, or nitrogen-linked substituents including amine, methylamine, dimethylamine or higher secondary or tertiary alkyl or aryl amines, or oxygen-, sulfur- and selenium-linked substituents including hydroxyl, sulfhydryl and selenylhydryl and alkyl and aryl ether analogs thereof, or silicon-, phosphorous- or boron-linked substituents including alkyl or aryl substitutions at these atoms. In all cases, linking atoms and linking-atom substituents are as required for valence satisfaction and chemical stability. In certain embodiments, -A- may be any disubstituted residue, such as oxygen or sulfur, or a trisubstituted residue, such as nitrogen, or a tetrasubstituted residue, such as carbon, or any other residue capable of forming two or more stable bonds; furthermore, M₅, M₆, M₇, M₈, M₉, M₁₀, M₁₁, M₁₂, M₁₃ and M₁₄ are each independently selected from the group consisting of carbon, nitrogen, sulfur, oxygen atoms and/or an atomic null such that a monocyclic or bicyclic ring system providing valence satisfaction and chemical stability are achieved; furthermore, single and multiple substitutions by the substituent(s) Y at positions M₅-M₁₄ on the above designated carbon, nitrogen and sulfur atoms may be hydrogen or halogen, such as fluorine, chlorine or bromine, or substituted oxygen-, carbon-, nitrogen- or sulfur-linked substituents including methyl, ethyl, isopropyl, higher alkyl and aryl substituents, hydroxyl, sulfhydryl, alkyl and aryl ethers and thioethers, amine, methyl amine, dimethyl amine and higher alkyl and aryl secondary and tertiary amines such that valence satisfaction and chemical stability are achieved. R₂ and R₃ are each independently chosen to be hydrogen, oxygen-, carbon-, nitrogen- or sulfur-linked substituents including methyl, ethyl, isopropyl, higher alkyl and aryl substituents, hydroxyl, sulfhydryl, alkyl and aryl ethers and thioethers, amine, methyl amine, dimethyl amine and higher alkyl and aryl secondary and tertiary amines for valence satisfaction; R₂ and R₃ may be covalently linked to give a set of monocyclic aza-cycles. R₄ and R_{4′} may be independently hydrogen, carbon-, oxygen-, nitrogen- or sulfur-linked substituents including methyl, ethyl, isopropyl, higher alkyl and aryl substituents, hydroxyl, sulfhydryl, alkyl and aryl ethers and thioethers, amine, methyl amine, dimethyl amine and higher alkyl and aryl secondary and tertiary amines. In all cases, substitutions are chosen such that valence satisfaction and chemical stability are achieved. R₄ and R_{4′} may be covalently linked to give a set of cyclic compounds.

In another set of embodiments, the composition has a structure:

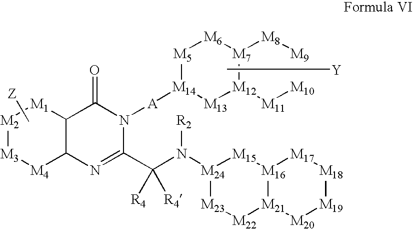

where M₁, M₂, M₃ and M₄ are each independently selected from the group consisting of carbon, nitrogen, sulfur, oxygen residue and/or an atomic null such that a fused bicyclic ring system providing valence satisfaction and chemical stability is achieved. Single and multiple substitutions by the substituent(s) Z at positions M₁-M₄ on the above designated carbon, nitrogen and sulfur atoms may be hydrogen or halogen, such as fluorine, chlorine or bromine, or carbon-linked substituents such as methyl, ethyl, propyl, isopropyl and higher alkyl and aryl analogs, or nitrogen-linked substituents including amine, methylamine, dimethylamine or higher secondary or tertiary alkyl or aryl amines, or oxygen-, sulfur- and selenium-linked substituents including hydroxyl, sulfhydryl and selenylhydryl and alkyl and aryl ether analogs thereof, or silicon-, phosphorus- or boron-linked substituents including alkyl or aryl substitutions at these residues. In all cases, linking residues and linking-atom substituents are as required for valence satisfaction and chemical stability. In certain embodiments, -A- may be any disubstituted residue, such as oxygen or sulfur, or a trisubstituted residue, such as nitrogen, or a tetrasubstituted residue, such as carbon, or any other atom capable of forming two or more stable bonds; furthermore, M₅, M₆, M₇, M₈, M₉, M₁₀, M₁₁, M₁₂, M₁₃ and M₁₄ are each independently selected from the group consisting of carbon, nitrogen, sulfur, oxygen atoms and/or an atomic null such that a monocyclic or bicyclic ring system providing valence satisfaction and chemical stability are achieved. Single and multiple substitutions by the substituent(s) Y at positions M₅-M₁₄ on the above designated carbon, nitrogen and sulfur atoms may be hydrogen or halogen, such as fluorine, chlorine or bromine, or substituted oxygen-, carbon-, nitrogen- or sulfur-linked substituents including methyl, ethyl, isopropyl, higher alkyl and aryl substituents, hydroxyl, sulfhydryl, alkyl and aryl ethers and thioethers, amine, methyl amine, dimethyl amine and higher alkyl and aryl secondary and tertiary amines such that valence satisfaction and chemical stability are achieved. R₂ and R₃ are each independently chosen to be hydrogen, oxygen-, carbon-, nitrogen- or sulfur-linked substituents including methyl, ethyl, isopropyl, higher alkyl and aryl substituents, hydroxyl, sulfhydryl, alkyl and aryl ethers and thioethers, amine, methyl amine, dimethyl amine and higher alkyl and aryl secondary and tertiary amines for valence satisfaction. R₂ and R₃ may be covalently linked to give a set of monocyclic aza-cycles. In certain embodiments, R₂ is a moiety containing two residues other than hydrogen and no more than eight residues other than hydrogen. M₁₅-M₂₄ may be independently carbon, nitrogen, oxygen or sulfur or any residue other than hydrogen or halogen and, in certain embodiments, may be either a moiety where M₁₅, M₁₆, M₁₇, M₁₈, M₁₉, M₂₀, M₂₁, M₂₂, M₂₃ and M₂₄ are each independently selected from the group consisting of carbon, nitrogen, sulfur, oxygen residue and/or an atomic null such that a monocyclic or bicyclic ring system providing valence satisfaction and chemical stability are achieved. R₄ and R_{4′} may be independently hydrogen, carbon, oxygen, nitrogen or sulfur with substitutions as needed for valence satisfaction; R₄ and R_{4′} may be covalently linked to give a set of cyclic compounds.

In another set of embodiments, the composition has a structure:

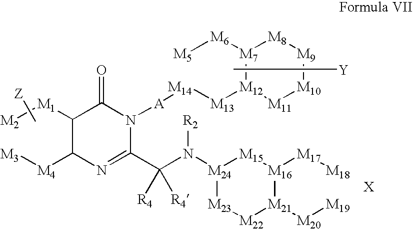

where M₁, M₂, M₃ and M₄ are each independently selected from the group consisting of carbon, nitrogen, sulfur, oxygen atoms and/or an atomic null such that a fused bicyclic ring system providing valence satisfaction and chemical stability is achieved. Single and multiple substitutions by the substituent(s) Z at positions M₁-M₄ on the above designated or bromine, or carbon-linked substituents such as methyl, ethyl, propyl, isopropyl and higher alkyl and aryl analogs, or nitrogen-linked substituents including amine, methylamine, dimethylamine or higher secondary or tertiary alkyl or aryl amines, or oxygen-, sulfur- and selenium-linked substituents including hydroxyl, sulfhydryl and selenylhydryl and alkyl and aryl ether analogs thereof, or silicon-, phosphorus- or boron-linked substituents including alkyl or aryl substitutions at these atoms. In all cases, linking residues and linking-atom substituents are as required for valence satisfaction and chemical stability. In certain embodiments, -A- may be any disubstituted residue, such as oxygen or sulfur, or a trisubstituted residue, such as nitrogen, or a tetrasubstituted residue, such as carbon, or any other residuecapable of forming two or more stable bonds; furthermore, M₅, M₆, M₇, M₈, M₉, M₁₀, M₁₁, M₁₂, M₁₃ and M₁₄ are each independently selected from the group consisting of carbon, nitrogen, sulfur, oxygen residue and/or an atomic null such that a monocyclic or bicyclic ring system providing valence satisfaction and chemical stability are achieved. Single and multiple substitutions by the substituent(s) Y at positions M₅-M₁₄ on the above designated carbon, nitrogen and sulfur residue may be hydrogen or halogen, such as fluorine, chlorine or bromine, or substituted oxygen-, carbon-, nitrogen- or sulfur-linked substituents including methyl, ethyl, isopropyl, higher alkyl and aryl substituents, hydroxyl, sulfhydryl, alkyl and aryl ethers and thioethers, amine, methyl amine, dimethyl amine and higher alkyl and aryl secondary and tertiary amines such that valence satisfaction and chemical stability are achieved. R₂ and R₃ are each independently chosen to be hydrogen, oxygen-, carbon-, nitrogen- or sulfur-linked substituents including methyl, ethyl, isopropyl, higher alkyl and aryl substituents, hydroxyl, sulfhydryl, alkyl and aryl ethers and thioethers, amine, methyl amine, dimethyl amine and higher alkyl and aryl secondary and tertiary amines for valence satisfaction. R₂ and R₃ may be covalently linked to give a set of monocyclic aza-cycles. In certain embodiments, R₂ is a moiety containing two residues other than hydrogen and no more than eight residues other than hydrogen. M₁₅-M₂₄ may be independently carbon, nitrogen, oxygen or sulfur or any residue other than hydrogen or halogen and, in certain embodiments, may be either a moiety where M₁₅, M₁₆, M₁₇, M₁₈, M₁₉, M₂₀,M₂₁, M₂₂, M₂₃ and M₂₄ are each independently selected from the group consisting of carbon, nitrogen, sulfur, oxygen residues and/or an atomic null such that a monocyclic or bicyclic ring system providing valence satisfaction and chemical stability are achieved. Single and multiple substitutions by substituent(s) X at positions M₅-M₂₄ on the above designated carbon, nitrogen and sulfur atoms may be hydrogen or halogen, such as fluorine, chlorine or bromine, or substituted oxygen-, carbon-, nitrogen- or sulfur-linked substituents including methyl, ethyl, isopropyl, higher alkyl and aryl substituents, hydroxyl, sulfhydryl, alkyl and aryl ethers and thioethers, amine, methyl amine, dimethyl amine and higher alkyl and aryl secondary and tertiary amines such that valence satisfaction and chemical stability are achieved. R₂ and R₃ are each independently chosen to be hydrogen, oxygen-, carbon-, nitrogen- or sulfur-linked substituents including methyl, ethyl, isopropyl, higher alkyl and aryl substituents, hydroxyl, sulfhydryl, alkyl and aryl ethers and thioethers, amine, methyl amine, dimethyl amine and higher alkyl and aryl secondary and tertiary amines for valence satisfaction; R₂ and R₃ may be covalently linked to give a set of monocyclic aza-cycles. R₄ and R_{4′} may be independently hydrogen, carbon-, oxygen-, nitrogen- or sulfur-linked substituents including methyl, ethyl, isopropyl, higher alkyl and aryl substituents, hydroxyl, sulfhydryl, alkyl and aryl ethers and thioethers, amine, methyl amine, dimethyl amine and higher alkyl and aryl secondary and tertiary amines. In all cases, substitutions are chosen such that valence satisfaction and chemical stability are achieved. R₄ and R_{4′} may be covalently linked to give a set of cyclic compounds.

In another set of embodiments, the composition has a structure:

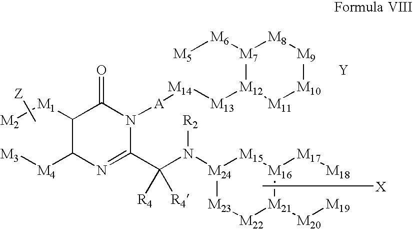

where M₁, M₂, M₃ and M₄ are each independently selected from the group consisting of carbon, nitrogen, sulfur, oxygen residue and/or an atomic null such that a fused bicyclic ring system providing valence satisfaction and chemical stability is achieved. Preferably, M₁-M₄ may be carbon residues yielding a quinazolinone core structure. Single and multiple substitutions by the substituent(s) Z at positions M₁-M₄ on the above designated carbon, nitrogen and sulfur residues may be hydrogen or halogen, such as fluorine, chlorine or bromine, or carbon-linked substituents such as methyl, ethyl, propyl, isopropyl and higher alkyl and aryl analogs, or nitrogen-linked substituents including amine, methylamine, dimethylamine or higher secondary or tertiary alkyl or aryl amines, or oxygen-, sulfur- and selenium-linked substituents including hydroxyl, sulfhydryl and selenylhydryl and alkyl and aryl ether analogs thereof, or silicon-, phosphorus- or boron-linked substituents including alkyl or aryl substitutions at these atoms. In all cases, linking atoms and linking-atom substituents are as required for valence satisfaction and chemical stability. Preferably, M₃ is a carbon atom substituted with chlorine (Z). In certain embodiments, -A- may be any disubstituted residue, such as oxygen or sulfur, or a trisubstituted residue, such as nitrogen, or a tetrasubstituted residue, such as carbon, or any other residue capable of forming two or more stable bonds; furthermore, M₅, M₆, M₇, M₈, M₉, M₁₀, M₁₁, M₁₂, M₁₃ and M₁₄ are each independently selected from the group consisting of a carbon, nitrogen, sulfur, oxygen residue and/or an atomic null such that a monocyclic or bicyclic ring system providing valence satisfaction and chemical stability are achieved. Single and multiple substitutions by the substituent(s) Y at positions M₅-M₁₄ on the above designated carbon, nitrogen and sulfur atoms may be hydrogen or halogen, such as fluorine, chlorine or bromine, or substituted oxygen-, carbon-, nitrogen- or sulfur-linked substituents including methyl, ethyl, isopropyl, higher alkyl and aryl substituents, hydroxyl, sulfhydryl, alkyl and aryl ethers and thioethers, amine, methyl amine, dimethyl amine and higher alkyl and aryl secondary and tertiary amines such that valence satisfaction and chemical stability are achieved. Preferably, the substituent (—Y-M₅-M₁₄) is a benzyl group. R₂ and R₃ are each independently chosen to be hydrogen, oxygen-, carbon-, nitrogen- or sulfur-linked substituents including methyl, ethyl, isopropyl, higher alkyl and aryl substituents, hydroxyl, sulfhydryl, alkyl and aryl ethers and thioethers, amine, methyl amine, dimethyl amine and higher alkyl and aryl secondary and tertiary amines for valence satisfaction. R₂ and R₃ may be covalently linked to give a set of monocyclic aza-cycles. R₂ may be a moiety containing two residues other than hydrogen and no more than eight residues other than hydrogen. Preferably, R₂ is a propyl amine (—(CH₂)₃—NH₂) group. In certain embodiments, -B- may be any disubstituted residue, such as oxygen or sulfur, or a trisubstituted residue, such as nitrogen, or a tetrasubstituted residue, such as carbon, or any other residue capable of forming two or more stable bonds; linker -B- may be chosen as an atomic null through an octa-atomic set of non-hydrogen atoms such that valence satisfaction and chemical stability are achieved. Furthermore, M₁₅-M₂₄ may be independently carbon, nitrogen, oxygen or sulfur or any residue other than hydrogen or halogen and, in certain embodiments, may be either a moiety where M₁₅, M₁₆, M₁₇, M₁₈, M₁₉, M₂₀, M₂₁, M₂₂, M₂₃ and M₂₄ are each independently selected from the group consisting of a carbon, nitrogen, sulfur, oxygen residue and/or an atomic null such that a monocyclic or bicyclic ring system providing valence satisfaction and chemical stability are achieved. Single and multiple substitutions by substituent(s) X at positions M₁₅-M₂₄ on the above designated carbon, nitrogen and sulfur residue may be hydrogen or halogen, such as fluorine, chlorine or bromine, or substituted oxygen-, carbon-, nitrogen- or sulfur-linked substituents including methyl, ethyl, isopropyl, higher alkyl and aryl substituents, hydroxyl, sulfhydryl, alkyl and aryl ethers and thioethers, amine, methyl amine, dimethyl amine and higher alkyl and aryl secondary and tertiary amines such that valence satisfaction and chemical stability are achieved. Preferably, (-B-M₁₅-M₂₄) is a 4-methyl benzoate substituent. R₄ and R_{4′} may be independently hydrogen, carbon-, oxygen-, nitrogen- or sulfur-linked substituents including methyl, ethyl, isopropyl, higher alkyl and aryl substituents, hydroxyl, sulfhydryl, alkyl and aryl ethers and thioethers, amine, methyl amine, dimethyl amine and higher alkyl and aryl secondary and tertiary amines. In all cases, substitutions are chosen such that valence satisfaction and chemical stability are achieved. R₄ and R_{4′} may be covalently linked to give a set of cyclic compounds. Preferably, R₄ is a hydrogen, while R_{4′} is an isopropyl group.

In one aspect of the invention, the compound of Formula I to Formula VIII may be exemplified by Compounds Nos. 1-5, 7, 11, 13-15, 17-24, 26, 28-31, 42-48, 51, 55-106, 173, 174-179, and 184-186 as set forth in Tables 2 to 5.

In another aspect, the invention involves a composition comprising compounds of a general structure and formula that can be routinely prepared by well-established methods. In one set of embodiments, the compound has a structure:

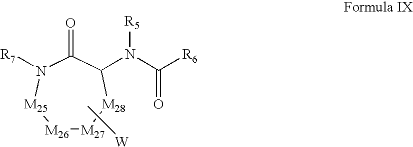

where M₂₅-M₂₈ are independently selected from the group consisting of carbon, nitrogen, sulfur, oxygen residue and/or an atomic null such that a mono-cyclic system of at least 4 residues and no more than 7 residues displaying valence satisfaction and chemical stability is achieved. Substitution(s) (W) at positions M₂₅-M₂₈ on the above residues may be hydrogen or halogen, such as fluorine, chlorine or bromine, or carbon-linked substituents such as methyl, ethyl, propyl, isopropyl and higher alkyl and aryl analogs, or nitrogen-linked substituents including amine, methylamine, dimethylamine or higher secondary or tertiary alkyl or aryl amines, or oxygen-, sulfur- and selenium-linked substituents including hydroxyl, sulfhydryl and selenylhydryl and alkyl and aryl ether analogs thereof, or silicon-, phosphorous- or boron-linked substituents including alkyl or aryl substitutions at these residues. In all cases, linking atoms and linking-atom substituents are as required for valence satisfaction and chemical stability. R₅ may be any carbon-linked moiety including methyl, ethyl, benzyl, aryl and substituted analogs thereof. R₆ is independently chosen to be a carbon- or nitrogen-linked substituent such that valence satisfaction and chemical stability are achieved. R₇ is hydrogen or a carbon-linked substituent such as methyl, ethyl, isopropyl, higher alkyl, benzyl or aryl and substituted analogs thereof.

Preferred compositions include the formula M₂₅-M₂₈ as all saturated carbon atoms giving a 7-membered cyclic compound where R₅ is 4-benzyloxybenzyl, R₆ is 2-chlorophenyl-1-aminocarbonyl and R₇ is hydrogen. In another preferred composition, the formula M₂₅-M₂₈ exists as all saturated carbon atoms giving a 7-membered cyclic compound while R₅ is 3-methoxy-4-benzyloxybenzyl, R₆ is benzyl and R₇ is hydrogen.

In one aspect of the invention, the compound of Formula IX may be exemplified by Compounds Nos. 33, 50, 166-172, 180-183, and 188 as set forth in Tables 2 to 5.

In another aspect, the invention is directed to a composition comprising compounds of a general structure and formula that can be routinely prepared by well-established methods. In one set of embodiments, the compound has a structure:

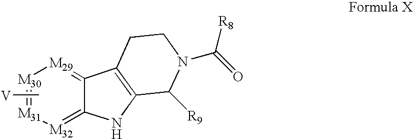

where M₂₉-M₃₂ are independently selected from the group consisting of carbon, nitrogen, sulfur, oxygen residue and/or an atomic null such that a tri-cyclic system displaying valence satisfaction and chemical stability is achieved. Substitution(s) (V) at positions M₂₉-M₃₂ on the above atoms may be hydrogen or halogen, such as fluorine, chlorine or bromine, or carbon-linked substituents such as methyl, ethyl, propyl, isopropyl and higher alkyl and aryl analogs, or nitrogen-linked substituents including amine, methylamine, dimethylamine or higher secondary or tertiary alkyl or aryl amines, or oxygen-, sulfur- and selenium-linked substituents including hydroxyl, sulfhydryl and selenylhydryl and alkyl and aryl ether analogs thereof, or silicon-, phosphorous- or boron-linked substituents including alkyl or aryl substitutions at these atoms. In all cases, linking atoms and linking-atom substituents are as required for valence satisfaction and chemical stability. R₈ may be any carbon- or nitrogen-linked moiety including methyl, ethyl, benzyl, aryl and substituted analogs thereof. R₉ is independently chosen to be a carbon-linked substituent such as methyl, ethyl, isopropyl, higher alkyl, benzyl or aryl and substituted analogs thereof. R₈ and R₉ may be covalently joined to give a cyclic structure.

Preferred compounds include the formula M₂₉-M₃₂ as carbon atoms giving a tricyclic compound where R₈ is cyclopentyl and R₉ is 4-trifluoromethylbenzene. In another preferred composition, the formula M₂₉-M₃₂ exists as carbon atoms giving a tricyclic compound wherein R₈ is 4-methylbenzene and R₉ is 4-methylbenzene.

In one aspect of the invention, the compound of Formula X may be exemplified by Compounds Nos. 8, 25, 115, 118, 120, 122-128, 130-132, 134 as set forth in Tables 2 to 5.

In another aspect, the invention involves a composition comprising compounds of a general structure and formula that can be routinely prepared by well-established methods. In one set of embodiments, the compound has a structure:

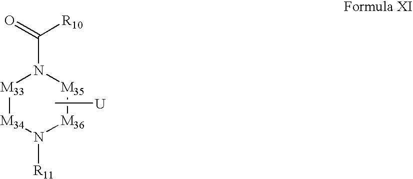

where M₃₃-M₃₆ are selected from the group consisting of carbon or nitrogen, and/or atomic null(s) such that a monocyclic or acyclic system displaying valence satisfaction and chemical stability is achieved. Substitution(s) (U) at positions M₃₃-M₃₆ on the above residues may be hydrogen or carbon-linked substituents such as methyl, ethyl, propyl, isopropyl and higher alkyl and aryl analogs, or nitrogen-linked substituents including amine, methylamine, dimethylamine or higher secondary or tertiary alkyl or aryl amines, or oxygen-, sulfur- and selenium-linked substituents including hydroxyl, sulfhydryl and selenylhydryl and alkyl and aryl ether analogs thereof, or silicon-, phosphorus- or boron-linked substituents including alkyl or aryl substitutions at these atoms. In all cases, linking atoms and linking-atom substituents are as required for valence satisfaction and chemical stability. R₁₀ may be any carbon- or nitrogen-linked moiety including methyl, ethyl, benzyl, aryl and substituted analogs thereof. R₁₁ is independently chosen to be a carbon-linked substituent such as methyl, ethyl, ethyl amine, isopropyl, higher alkyl, benzyl or aryl and substituted analogs thereof.

Preferred compositions include the formula M₃₃-M₃₆ as saturated carbon residues giving a monocyclic compound where R₁₀ is 2,4-dimethoxybenzoate and R₁₁ is 2-amino-(N-4-fluorobenzyl-N-4-fluorobenzoyl)ethyl. In another preferred composition, the formula M₃₃-M₃₆ as saturated carbon residues giving a monocyclic compound where R₁₀ is 2-carboxythiophenyl and R₁₁ is 2-amino-(N-4-fluorobenzyl-N-carboxyamino tert-butyl)ethyl.

In one aspect of the invention, the compound of Formula XI may be exemplified by Compounds Nos. 35, and 107-114 as set forth in Tables 2 to 5.

In another aspect, the invention involves a composition comprising compounds of a general structure and formula that can be routinely prepared by well-established methods. In one set of embodiments, the compound has a structure:

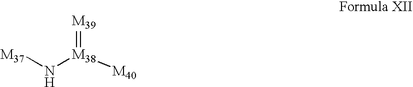

where M₃₇ is selected from the group consisting of substituted carbon, M₃₈ is selected from the group consisting of carbon and sulfur and M₃₉ is selected from either oxygen or sulfur and M₄₀ is selected from either substituted carbon or substituted nitrogen to provide a system displaying valence satisfaction and chemical stability. M₃₇ and M₄₀ may be covalently joined to provide a cyclic system. Substitution(s) at positions M₃₇ and M₄₀ on the above residues may be hydrogen or carbon-linked substituents such as methyl, ethyl, propyl, isopropyl and higher alkyl and aryl analogs, or nitrogen-linked substituents including amine, methylamine, dimethylamine or higher secondary or tertiary alkyl or aryl amines, or oxygen-, sulfur- and selenium-linked substituents including hydroxyl, sulfhydryl and selenylhydryl and alkyl and aryl ether analogs thereof, or silicon-, phosphorus- or boron-linked substituents including alkyl or aryl substitutions at these residues. In all cases, linking atoms and linking-atom substituents are as required for valence satisfaction and chemical stability.

In one aspect of the invention, the compound of Formula XII may be exemplified by Compounds Nos. 6, 9, 10, 12, 16, 27, 32, 34, 36-41, 49, 52-54, 116, 117, 119, 121, 129, 133,

In certain embodiments of the invention, whether such embodiments involve a composition, composition including pharmaceutical carrier, or method of making or using a composition, each of such embodiments includes any composition disclosed herein.

Other advantages, novel features, and objects of the invention will become apparent from the following detailed description of non-limiting embodiments of the invention In cases where the present specification and a document incorporated by reference include conflicting disclosure, the present specification shall control.

In one aspect, the invention is defined, at least in part, by compositions having certain structures, as further described below. In these structures, the term “chemical bond” refers to any type of chemical bond, for example, a covalent bond, an ionic bond, a hydrogen bond, a van der Waals bond, a metal ligand bond, a dative bond, a hydrophobic interaction, or the like. It is to be understood that all compositions are useful or potentially useful for any of the methods of treatment described herein.

In these structures, atoms able to form at least three covalent bonds include those atoms of the carbon family (e.g., carbon, silicon, or germanium), the nitrogen family (e.g., nitrogen, phosphorus, or arsenic), or the boron family (e.g., boron, aluminum, or gallium). In some embodiments, the atoms able to form at least three covalent bonds found within structures of the invention are carbon, nitrogen, silicon, and phosphorus, and in certain embodiments, the atoms are carbon and nitrogen.

The term “halogen,” or equivalently, “halogen atom,” is given its ordinary meaning as used in the field of chemistry. The halogens include fluorine, chlorine, bromine, iodine, and astatine. Preferably, the halogen atoms used in the present invention include one or more of fluorine, chlorine, bromine, or iodine. In certain embodiments of the invention, the halogen atoms found within the structure are fluorine, chlorine, and bromine; fluorine and chlorine; chlorine and bromine, or a single type of halogen atom.

As used herein, a “saturated” bond is given its ordinary meaning as used in the field of chemistry. A saturated moiety generally does not contain any double, triple, or higher order chemical bonds in its structure. The saturated moiety can contain any number or types of atoms (e.g., oxygen, carbon, nitrogen, hydrogen, or halogen atoms) in any configuration, so long as the moiety contains only single bonds between the atoms. For example, the saturated moiety may be an aliphatic structure or a cyclic structure. A saturated moiety may be connected to a parent structure at one or more points. Examples of saturated moieties include:

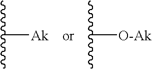

which each are connected to a parent structure at one point, or:

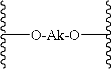

which is connected to a parent structure at more than one point (in this example, using ether linkages). In these structures, “Ak” refers to an alkyl group, as described below. As one example, the alkyl group in these structures may have one, two, three, or four carbon atoms, and may be straight-chained or branched, as long as no double or triple bonds are present. The alkyl group may also include only hydrogen atoms, or include halogen atoms as well.

Conversely, an “unsaturated” moiety is a moiety that contains at least one higher-order chemical bond within its structure, i.e., at least one double bond or triple bond between two atoms within its structure. The unsaturated moiety may contain, in some cases, more than one double and/or triple bond within its structure, for example, as in an alkadiene or an alkenyne.

As used herein, an “alkyl” is given its ordinary meaning as used in the field of organic chemistry. Alkyl or aliphatic groups typically contains any number of carbon atoms, for example, between 1 and 20 carbon atoms, between 1 and 15 carbon atoms, between I and 10 carbon atoms, or between 1 and 5 carbon atoms. In some embodiments, the alkyl group will contain at least 1 carbon atom, at least 2 carbon atoms, at least 3 carbon atoms, at least 4 carbon atoms, at least 5 carbon atoms, at least 6 carbon atoms, at least 7 carbon atoms, or at least 8 carbon atoms. Typically, an alkyl group is a non-cyclic structure. In certain embodiments, the alkyl group is a methyl group or an ethyl group.

The carbon atoms may be arranged in any configuration within the alkyl moiety, for example, as a straight chain (i.e., a n-alkyl such as methyl, ethyl, propyl, butyl, pentyl, hexyl, heptyl, octyl, nonyl, decyl, or undecyl) or a branched chain, for example, a t-butyl group, or an isoalkyl group such as isopropyl, isobutyl, ispentanyl, or isohexanyl. The alkyl moiety may contain none or any number of double or triple bonds within its structure, for example, as in an alkene, an alkyne, an alkadiene, an alkadiyne, an alkenyne, etc.

The alkyl group may contain any number of substituents. For example, the alkyl group may contain a halogen, an alkoxy (e.g., a methoxy, an ethoxy, a propoxy, an isopropoxy, a butoxy, a pentoxy, or the like), an amine (e.g., a primary, secondary, or tertiary amine, for example, an dimethylamine ethyl group), or a hydroxide as a substituent. As one example, if the alkyl group is a methyl group, then the methyl group may be substituted to form, for instance, a halogenated methyl group such as chloromethyl, bromomethyl, or iodomethyl. In some embodiments of the invention, more than one substituent may be present. For example, the alkyl group may have two or more halogen atoms (for example, two chlorine atoms, or a chlorine and a bromine atom), a halogen and an alkoxy group, or the like.

In some embodiments of the invention, the alkyl group may also contain one or more heteroatoms substituted within the alkyl group, such as a nitrogen atom (e.g., as in an amine such as a primary, secondary, or tertiary amine) or an oxygen atom (as in an ether moiety). However, in other embodiments of the invention, the main chain of the alkyl group is free of heteroatoms and includes carbon atoms. As used herein, the term “heteroatoms” refers to atoms that can replace carbon atoms within an alkyl group without affecting the connectivity of the alkyl group; these typically include oxygen and nitrogen atoms. Halogen atoms and hydrogen atoms are not considered to be heteroatoms; for example, a chlorine atom can replace a hydrogen atom within an alkyl group without affecting the connectivity of the alkyl group As used herein, a “non-heteroatom alkyl group” is an alkyl group which does not contain any atoms at the carbon positions other than carbon. Some structures are defined as being free of non-terminal heteroatoms. As used herein, a “non-terminal” atom is an atom within a structure that is connected to at least two different atoms having a valency greater than 1 (e.g., the atom is connected to two non-hydrogen and non-halogen atoms). For example, the oxygen in —CH₂—OH and the nitrogen atom in —CH₂—NH₂ are not connected to two different atoms having a valency greater than 1, and thus are not non-terminal heteroatoms.

Similarly, a “cyclic” structure, as used herein, is given its ordinary definition in the field of organic chemistry, i.e., a structure that contains at least one ring of atoms, and may contain more than one ring of atoms. In other words, a cyclic structure has at least one chain of atoms that does not have a terminal end. The chain may have, for example, three, four, five, six, seven, or more atoms arranged to form a ring. The atoms within the chain may be carbon atoms, nitrogen atoms, oxygen atoms, silicon atoms, or any other atom that is able to bond to at least two different atoms.

In some embodiments of the invention, one or more substituents may be present on the cyclic structure. The substituents may be any substituent, as previously described in connection with alkyl moieties, for example, a halogen, an alkoxy, an amine, a hydroxide, or the like. In some embodiments, the substituents may also be alkyl groups, as previously described, for example, a methyl group, an ethyl group, a propyl group, and the like.

The cyclic structure may have one or more heteroatoms in some embodiments. For example, the cyclic structure may include a cyclohexane or a cyclopentane ring having one or more heteroatoms, such as:

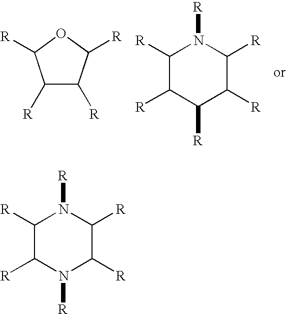

where the R's indicate the presence of additional atoms or substituents. The atoms substituted within the cyclohexane ring are able to form at least three covalent bonds, and, if able to form four covalent bonds, the fourth covalent bond may be attached to any atom.

The cyclic structure may be a saturated cyclic structure (such as a cyclohexyl or a cyclopentyl structure), or an unsaturated cyclic structure (such as a cyclohexenyl structure or an aromatic structure). Examples of aromatic structures, include, for instance, phenyl, naphthalenyl, anthacenyl, tolyl, pyridinyl, furanyl, pyrrolyl, and the like. A “nonaromatic cyclic structure” is a structure in which aromaticity of the cyclic structure is not present (for example, as in a saturated cyclic structure, a cycloalkenyl moiety, etc.)

In one set of embodiments, the aromatic structure includes a benzene ring. If substituents are present on the benzene ring (as previously discussed, for example, a halogen atom, a methyl group, a methoxy group, a trifluoromethyl group, etc.), they may be located in any position, i.e., in any ortho, meta, or para position, relative to the point of attachment of the benzene ring. If more than one substituent is present, then the substituents may be located at any available point within the benzene ring. For example, if there are two substituents, they may be located in the ortho and meta positions (i.e., in the 2,3 or 2,5 positions), the ortho and para positions, in the two ortho positions, in the two meta positions, or in the meta and para positions.

In one set of embodiments, the aromatic group is a nonsubstituted aromatic group, for example, a phenyl or a naphthalenyl group. In another set of embodiments, the aromatic structure is a halophenyl group or a dihalophenyl group, for example, 3-chloro-4-flurophenyl; o-, in-, orp-chlorophenyl; 2,4-difluorophenyl; or o-, m-, orp-bromophenyl. In another set of embodiments, the aromatic structure is a methylphenyl or a dimethyl phenyl group, for example, o-, m-, orp-methylphenyl; 2,3-dimethylphenyl; 2,4-dimethylphenyl; 2,5-dimethylphenyl. In another set of embodiments, the aromatic group is an alkylphenyl group, such as o-, m-, orp-methylphenyl; o-, m-, orp-ethylphenyl; 2-phenylethyl, or benzyl. In another set of embodiments, the aromatic structure is a halomethylphenyl group, such as 3-chloro-2-methylphenyl. In another set of embodiments, the aromatic structure is an alkoxyphenyl or a dialkoxyphenyl group, for example, o-, m-, orp-isopropoxyphenyl; o-, m-, orp-methoxyphenyl; o-, m-, orp-ethoxyphenyl; or 2,4-dimethoxyphenyl. In one set of embodiments, the aromatic group is fused with another ring of atoms. The second ring may be aromatic or nonaromatic. Examples include:

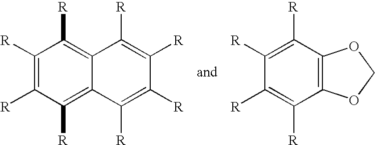

where the R's indicate the presence of additional atoms or substituents.

If the cyclic structure has more than one ring of atoms, the rings may be distributed in any manner within the moiety. For example, the two rings may not share a common atom, share only one common atom (e.g., as in a spiro-structure), or share more than one atom, as in a bicyclic structure or a propellane structure. If the two rings share at least one common chemical bond between two atoms, then the rings may be considered to be “fused.”

One example of a fused ring system is a structure:

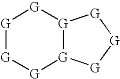

where a five member ring is fused to a six member ring in a bicyclic arrangement, and G represents atoms each having at least three covalent bonds, as previously discussed. In some embodiments, one or both rings may be aromatic. As one example, a single nitrogen substitution onto the five-member ring, when both rings are aromatic, can result in an indole moiety, for example:

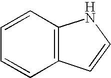

Additionally, other substituents or cyclic rings may be substituted onto the structure as well, for example, a cyclohexyl moiety.

If several rings are jointly fused to each other, then the rings may be considered to be “multifused.” One example of a multifused compound is an adamantane structure:

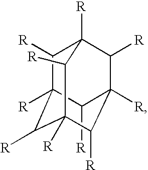

where the R's indicate the presence of additional atoms or substituents.

As used herein, when two cyclic groups are in a “branched configuration,” the two cyclic groups are on different branches of a common moiety. In other words, the two cyclic groups are not serially arranged relative to each other. That is, removal of either of the cyclic structures within the moiety does not automatically cause the other cyclic structure to be disconnected from the rest of the moiety. One example of this is illustrated by a diphenylmethyl moiety:

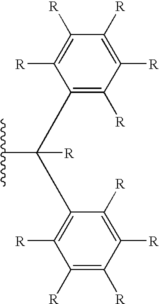

where the R's indicate the presence of additional atoms or substituents.

In one set of embodiments, the composition includes a substituted urea moiety. The substituted urea moiety includes at least one cyclic structure having at least seven members. In some cases, the cyclic structure may be a substituted cyclic structure, for example, the structure may include an azepane moiety or a cycloheptane structure, or the structure may include a cycloalkone moiety, that is, an oxygen atom that is double bonded to a member of the cyclic ring.

An “amino acid” is given its ordinary meaning as used in the field of biochemistry. An amino acid typically has a structure:

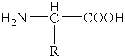

In this structure, R may be any suitable moiety. For example, R may be a hydrogen atom, a methyl group, or an isopropyl group. As used herein, the “natural amino acids” are the 20 amino acids commonly found in nature, i.e., alanine, arginine, asparagine, aspartic acid, cysteine, glutamine, glutamic acid, glycine, histidine, isoleucine, leucine, lysine, methionine, phenylalaine, proline, serine, threonine, tryptophan, tyrosine, and valine. Similarly, an unnatural amino acid is an amino acid, where the R group does not correspond to one of the natural amino acids.

In one embodiment, the compositions further comprise homologs, analogs, derivatives, enantiomers and functionally equivalent compositions thereof of the compositions of the invention, for example, compositions 1-188. Such homologs, analogs, derivatives, enantiomers and functionally equivalent compositions thereof of the that are able to detect or treat cancer, particularly MUC1-associated cancers. “Functionally equivalent” generally refers to a composition capable of treatment of patients having MUC1-associated cancer, or of patients susceptible to MUC1-associated cancers. It will be understood that the skilled artisan will be able to manipulate the conditions in a manner to prepare such homologs, analogs, derivatives, enantiomers and functionally equivalent compositions.

Homologs, analogs, derivatives, enantiomers and functionally equivalent compositions which are about as effective or more effective than the parent compound are also intended for use in certain embodiments of the methods of the invention. Such compositions may also be screened by the assays described herein for increased potency and specificity towards the cancer characterized by aberrant expression of MUC1, preferably with limited side effects. Synthesis of such compositions may be accomplished through typical chemical modification methods such as those routinely practiced in the art.

Another aspect of the present invention involves a method comprising providing any of the compositions of the present invention, and performing a combinatorial synthesis on the composition, preferably to obtain homologs, analogs, derivatives, enantiomers and functionally equivalent compositions thereof of the composition. An assay may be performed with the homolog, analog, derivative, enantiomer or functionally equivalent composition to determine its effectiveness in inhibiting cancer characterized by aberrant expression of MUC1. The combinatorial synthesis can involve subjecting a plurality of the compositions described herein to combinatorial synthesis.

### Formulations

Another aspect provides a method of administering any composition of the present invention to a subject. When administered, the compositions of the invention are applied in pharmaceutically acceptable amounts and as pharmaceutically acceptable compositions. Such preparations may routinely contain salts, buffering agents, preservatives, compatible carriers or other therapeutic ingredients. Examples of well-known carriers include glass, polystyrene, polypropylene, polyethylene, dextran, nylon, amylase, natural and modified cellulose, polyacrylamide, agarose and magnetite. The nature of the carrier can be either soluble or insoluble. Those skilled in the art will know of other suitable carriers, or will be able to ascertain such, using only routine experimentation.

In some cases, the present invention includes the step of bringing a composition of the invention into association or contact with a suitable carrier, which may constitute one or more accessory ingredients. The final compositions may be prepared by any suitable technique, for example, by uniformly and intimately bringing the composition into association with a liquid carrier, a finely divided solid carrier or both, optionally with one or more formulation ingredients such as buffers, emulsifiers, diluents, excipients, drying agents, antioxidants, preservatives, binding agents, chelating agents, or stabilizers and then, if necessary, shaping the product.

In some embodiments, the compositions of the present invention may be present as a pharmaceutically acceptable salt. The term “pharmaceutically acceptable salts” includes salts of the composition, prepared, for example, with acids or bases, depending on the particular substituents found within the composition and the treatment modality desired. Pharmaceutically acceptable salts can be prepared as alkaline metal salts, such as lithium, sodium, or potassium salts; or as alkaline earth salts, such as beryllium, magnesium or calcium salts. Examples of suitable bases that may be used to form salts include ammonium, or mineral bases such as sodium hydroxide, lithium hydroxide, potassium hydroxide, calcium hydroxide, magnesium hydroxide, and the like. Examples of suitable acids that may be used to form salts include inorganic or mineral acids such as hydrochloric, hydrobromic, hydroiodic, hydrofluoric, nitric, carbonic, monohydrogencarbonic, phosphoric, monohydrogenphosphoric, dihydrogenphosphoric, sulfuric, monohydrogensulfuric, phosphorous acids and the like. Other suitable acids include organic acids, for example, acetic, propionic, isobutyric, maleic, malonic, benzoic, succinic, suberic, fumaric, mandelic, phthalic, benzenesulfonic, p-tolylsulfonic, citric, tartaric, methanesulfonic, glucuronic, galactunoric, salicylic, formic, naphthalene-2-sulfonic, and the like. Still other suitable acids include amino acids such as arginate, aspartate, glutamate, and the like.

In general, pharmaceutically acceptable carriers for are well-known to those of ordinary skill in the art. As used herein, a “pharmaceutically acceptable carrier” refers to a non-toxic material that does not significantly interfere with the effectiveness of the biological activity of the active ingredient or ingredients. Pharmaceutically acceptable carriers include, for example, diluents, emulsifiers, fillers, salts, buffers, excipients, drying agents, antioxidants, preservatives, binding agents, bulking agents, chelating agents, stabilizers, solubilizers, and other materials well-known in the art. Examples of suitable formulation ingredients include diluents such as calcium carbonate, sodium carbonate, lactose, kaolin, calcium phosphate, or sodium phosphate; granulating and disintegrating agents such as corn starch or algenic acid; binding agents such as starch, gelatin or acacia; lubricating agents such as magnesium stearate, stearic acid, or talc; time-delay materials such as glycerol monostearate or glycerol distearate; suspending agents such as sodium carboxymethylcellulose, methylcellulose, hydroxypropylmethylcellulose, sodiumalginate, polyvinylpyrrolideone; dispersing or wetting agents such as lecithin or other naturally-occurring phosphatides; or thickening agents such as cetyl alcohol or beeswax. The compositions of the invention may be formulated into preparations in solid, semi-solid, liquid or gaseous forms such as tablets, capsules, elixrs, powders, granules, ointments, solutions, depositories, inhalants or injectables. The compositions of the present invention may be delivered by any suitable delivery method, for example, oral, parenteral or surgical administration. The invention also embraces locally administering the compositions of the invention, for example, as implants

Preparations include sterile aqueous or nonaqueous solutions, suspensions and emulsions. Examples of nonaqueous solvents are propylene glycol, polyethylene glycol, vegetable oil such as olive oil, an injectable organic esters such as ethyloliate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers, (such as those based on Ringer's dextrose), and the like. Preservatives and other additives may also be present such as, for example, antimicrobials, antioxidants, chelating agents and inert gases and the like. Those of skill in the art can readily determine the various parameters for preparing these pharmaceutical compositions without resort to undue experimentation.

The compositions of the invention may be administered singly or in combination with other compositions of the invention or other compositions. For example, in one embodiment, compositions of the invention are administered in combination with agents that block cell surface receptors, such as the alpha-V-beta-3 cell surface receptor.

According to certain embodiments of the methods of the invention, the compositions of the invention can be administered by injection by gradual infusion over time or by any other medically acceptable mode. Any medically acceptable method may be used to administer the composition to the patient. The particular mode selected will depend of course, upon factors such as the particular drug selected, the severity of the state of the subject being treated, or the dosage required for therapeutic efficacy. The methods of this invention, generally speaking, may be practiced using any mode of administration that is medically acceptable, meaning any mode that produces effective levels of the active composition without causing clinically unacceptable adverse effects.

The administration may be localized (i.e., to a particular region, physiological system, tissue, organ, or cell type) or systemic, depending on the condition to be treated. For example, the composition may be administered through parental injection, implantation, orally, vaginally, rectally, buccally, pulmonary, topically, nasally, transdermally, surgical administration, or any other method of administration where access to the target by the composition is achieved. Examples of parental modalities that can be used with the invention include intravenous, intradermal, subcutaneous, intracavity, intramuscular, intraperitoneal, epidural, or intrathecal. Examples of implantation modalities include any implantable or injectable drug delivery system. Oral administration may be preferred for some treatments because of the convenience to the patient as well as the dosing schedule. Compositions suitable for oral administration may be presented as discrete units such as capsules, pills, cachettes, tables, or lozenges, each containing a predetermined amount of the active compound. Other oral compositions include suspensions in aqueous or non-aqueous liquids such as a syrup, an elixir, or an emulsion.

The compositions of the present invention may be given in dosages, generally, at the maximum amount while avoiding or minimizing any potentially detrimental side effects. The compositions can be administered in effective amounts, alone or in a cocktail with other compounds, for example, other compounds that can be used to treat cancer. An effective amount is generally an amount sufficient to inhibit MUC1-associated cancer within the subject.

One of skill in the art can determine what an effective amount of the composition is by screening the ability of the composition using any of the assays described herein. The effective amounts will depend, of course, on factors such as the severity of the condition being treated; individual patient parameters including age, physical condition, size and weight; concurrent treatments; the frequency of treatment; or the mode of administration. These factors are well known to those of ordinary skill in the art and can be addressed with no more than routine experimentation. It is generally preferred that a maximum dose be used, that is, the highest safe dose according to sound medical judgment.

Dosages may be estimated based on the results of experimental models, optionally in combination with the results of assays of the present invention. Generally, daily oral prophylactic doses of active compounds will be from about 0.01 mg/kg per day to 2000 mg/kg per day. Oral doses in the range of 10 to 500 mg/kg, in one or several administrations per day, may yield suitable results. In the event that the response of a particular subject is insufficient at such doses, even higher doses (or effective higher doses by a different, more localized delivery route) may be employed to the extent that patient tolerance permits. Multiple doses per day are also contemplated in some cases to achieve appropriate systemic levels of the composition.

In administering the compositions of the invention to subjects, dosing amounts, dosing schedules, routes of administration and the like may be selected so as to affect other known activities of these compositions. For example, amounts, dosing schedules and routes of administration may be selected as described herein, whereby therapeutically effective levels for the inhibition or treatment of MUC1-associated cancers are provided, yet therapeutically effective levels for alternative treatments are not provided.

Other delivery systems suitable for use with the present invention include time-release, delayed release, sustained release, or controlled release delivery systems. Such systems may avoid repeated administrations of the active compounds of the invention in many cases, increasing convenience to the subject and the physician. Many types of release delivery systems are available and known to those of ordinary skill in the art. They include, for example, polymer based systems such as polylactic and/or polyglycolic acid, polyanhydrides, and polycaprolactone; nonpolymer systems that are lipid-based including sterols such as cholesterol, cholesterol esters, and fatty acids or neutral fats such as mono-, di- and triglycerides; hydrogel release systems; silastic systems; peptide based systems; wax coatings; compressed tablets using conventional binders and excipients; or partially fused implants. Specific examples include, but are not limited to, erosional systems in which the composition is contained in a form within a matrix, or diffusional systems in which an active component controls the release rate. The formulation may be as, for example, microspheres, hydrogels, polymeric reservoirs, cholesterol matrices, or polymeric systems. In some embodiments, the system may allow sustained or controlled release of the active compound to occur, for example, through control of the diffusion or erosion/degradation rate of the formulation. In addition, a pump-based hardware delivery system may be used in some embodiment of the invention.

Use of a long-term release implant may be particularly suitable in some cases. “Long-term release,” as used herein, means that the implant is constructed and arranged to deliver therapeutic levels of the composition for at least 30 or 45 days, and preferably at least 60 or 90 days, or even longer in some cases. Long-term release implants are well known to those of ordinary skill in the art, and include some of the release systems described above.

The present invention also provides any of the above-mentioned compositions useful for treatment of cancer characterized by aberrant expression of MUC1 packaged in kits, optionally including instructions for use of the composition for the treatment of cancer. That is, the kit can include a description of use of the composition for participation in any biological or chemical mechanism disclosed herein associated with cancer or tumor. The kits can further include a description of activity of cancer characterized by aberrant expression of MUC1 in treating the pathology, as opposed to the symptoms of the cancer. That is, the kit can include a description of use of the compositions as discussed herein. The kit also can include instructions for use of a combination of two or more compositions of the invention. Instructions also may be provided for administering the drug by any suitable technique, such as orally, intravenously, directly into the cerebrospinal fluid via a spinal drip, pump or implantable delivery device, or via another known route of drug delivery. Certain embodiments of the invention also involve promotion of the treatment of cancer characterized by aberrant expression of MUC1 according to any of the techniques and compositions and composition combinations described herein.

The compositions of the invention, in some embodiments, may be promoted for treatment of abnormal cell proliferation, cancers, or tumors, particularly MUC1-associated cancers or includes instructions for treatment of accompanying cell proliferation, cancers, or tumors, particularly MUC1-associated cancers as mentioned above. In another aspect, the invention provides a method involving promoting the prevention or treatment of cancer via administration of any one of the compositions of the present invention, and homologs, analogs, derivatives, enantiomers and functionally equivalent compositions thereof in which the composition is able to treat MUC1-associated cancers. As used herein, “promoted” includes all methods of doing business including methods of education, hospital and other clinical instruction, pharmaceutical industry activity including pharmaceutical sales, and any advertising or other promotional activity including written, oral and electronic communication of any form, associated with compositions of the invention in connection with treatment of cell proliferation, cancers or tumors. “Instructions” can define a component of promotion, and typically involve written instructions on or associated with packaging of compositions of the invention. Instructions also can include any oral or electronic instructions provided in any manner. The “kit” typically defines a package including any one or a combination of the compositions of the invention and the instructions, or homologs, analogs, derivatives, enantiomers and functionally equivalent compositions thereof, but can also include the composition of the invention and instructions of any form that are provided in connection with the composition in a manner such that a clinical professional will clearly recognize that the instructions are to be associated with the specific composition.

The kits described herein may also contain one or more containers, which can contain compounds such as the species, signaling entities, biomolecules and/or particles as described. The kits also may contain instructions for mixing, diluting, and/or administrating the compounds. The kits also can include other containers with one or more solvents, surfactants, preservative and/or diluents (e.g., normal saline (0.9% NaCl), or 5% dextrose) as well as containers for mixing, diluting or administering the components to the sample or to the patient in need of such treatment.

The compositions of the kit may be provided as any suitable form, for example, as liquid solutions or as dried powders. When the composition provided is a dry powder, the powder may be reconstituted by the addition of a suitable solvent, which may also be provided. In embodiments where liquid forms of the composition are used, the liquid form may be concentrated or ready to use. The solvent will depend on the compound and the mode of use or administration. Suitable solvents for drug compositions are well known and are available in the literature. The solvent will depend on the compound and the mode of use or administration.

The kit, in one set of embodiments, may comprise a carrier means being compartmentalized to receive in close confinement one or more container means such as vials, tubes, and the like, each of the container means comprising one of the separate elements to be used in the method. For example, one of the container means may comprise a positive control in the assay. Additionally, the kit may include containers for other components, for example, buffers useful in the assay.

TABLE 1
Peptide Sequences (Listed from N-terminus to C-terminus)

Histidine-Tagged Truncated receptor (His-TR) (having “SPY” sequence of
var-PSMGFR):

| | |
|---|---|
| GTINVHDVETQFNQYKTEAASPYNLTISDVSVSHHHHHH | (SEQ ID NO: 1) |

An example of a Histidine-Tagged Primary Sequence of the MUC1 Growth Factor
Receptor (His-var-PSMGFR) (having “SPY” sequence of var-PSMGFR):

| | |
|---|---|
| GTINVHDVETQFNQYKTEAASPYNLTISDVSVSDVPFPFSAQSGAHHHHHH | (SEQ ID NO: 2) |

Histidine-Tagged Extended Sequence of MUC1 Growth Factor Receptor (ESMGFR)
(having “SPY” sequence of var-PSMGFR):

| | |
|---|---|
| VQLTLAFREGTINVHDVETQFNQYKTEAASPYNLTISDVSVS | (SEQ ID NO: 3) |
| DVPFPFHHHHHH | |

Histidine-Tagged Primary Sequence of the Interchain binding Region
(His-PSIBR):

| | |
|---|---|
| HHHHHHGFLGLSNIKFRPGSVVVQLTLAFRE | (SEQ ID NO: 4) |

Histidine-Tagged Repeat Motif 2 (His-RM2):

| | |
|---|---|
| PDTRPAPGSTAPPAHGVTSAPDTRPAPGSTAPPAHGVTSAHHHHHH | (SEQ ID NO: 5) |

Truncated PSMGFR receptor (TR) (having “SPY” sequence of var-PSMGFR):

| | |
|---|---|
| GTINVHDVETQFNQYKTEAASPYNLTISDVSVS | (SEQ ID NO: 6) |

“SPY” functional variant of the native Primary Sequence of the MUC1 Growth
Factor Receptor having enhanced stability (var-PSMGFR - An example of
“PSMGFR”):

| | |
|---|---|
| GTINVHDVETQFNQYKTEAASPYNLTISDVSVSDVPFPFSAQSGA | (SEQ ID NO: 7) |

Primary Sequence of the Interchain Binding Region) (PSIBR):

| | |
|---|---|
| GFLGLSNIKFRPGSVVVQLTLAFRE | (SEQ ID NO: 8) |
| Repeat Motif 2 (RM2): | |
| PDTRPAPGSTAPPAHGVTSAPDTRPAPGSTAPPAHGVTSA | (SEQ ID NO: 9) |
| Full-length MUC1 Receptor | |

(Mucin 1 precursor, Genbank Accession number: P15941
MTPGTQSPFF LLLLLTVLTV VTGSGHASST PGGEKETSAT QRSSVPSSTE
KNAVSMTSSV LSSHSPGSGS STTQGQDVTL APATEPASGS AATWGQDVTS
VPVTRPALGS TTPPAHDVTS APDNKPAPGS TAPPAHGVTS APDTRPAPGS
TAPPAHGVTS APDTRPAPGS TAPPAHGVTS APDTRPAPGS TAPPAHGVTS
APDTRPAPGS TAPPAHGVTS APDTRPAPGS TAPPAHGVTS APDTRPAPGS
TAPPAHGVTS APDTRPAPGS TAPPAHGVTS APDTRPAPGS TAPPAHGVTS
APDTRPAPGS TAPPAHGVTS APDTRPAPGS TAPPAHGVTS APDTRPAPGS
TAPPAHGVTS APDTRPAPGS TAPPAHGVTS APDTRPAPGS TAPPAHGVTS
APDTRPAPGS TAPPAHGVTS APDTRPAPGS TAPPAHGVTS APDTRPAPGS
TAPPAHGVTS APDTRPAPGS TAPPAHGVTS APDTRPAPGS TAPPAHGVTS
APDTRPAPGS TAPPAHGVTS APDTRPAPGS TAPPAHGVTS APDTRPAPGS
TAPPAHGVTS APDTRPAPGS TAPPAHGVTS APDTRPAPGS TAPPAHGVTS
APDTRPAPGS TAPPAHGVTS APDTRPAPGS TAPPAHGVTS APDTRPAPGS
TAPPAHGVTS APDTRPAPGS TAPPAHGVTS APDTRPAPGS TAPPAHGVTS
APDTRPAPGS TAPPAHGVTS APDTRPAPGS TAPPAHGVTS APDTRPAPGS
TAPPAHGVTS APDTRPAPGS TAPPAHGVTS APDTRPAPGS TAPPAHGVTS
APDTRPAPGS TAPPAHGVTS APDTRPAPGS TAPPAHGVTS APDTRPAPGS
TAPPAHGVTS APDTRPAPGS TAPPAHGVTS APDTRPAPGS TAPPAHGVTS
APDTRPAPGS TAPPAHGVTS APDTRPAPGS TAPPAHGVTS APDNRPALGS
TAPPVHNVTS ASGSASGSAS TLVHNGTSAR ATTTPASKST PFSIPSHHSD
TPTTLASHST KTDASSTHHS SVPPLTSSNH STSPQLSTGV SFFFLSFHIS

| | |
|---|---|
| NLQFNSSLED PSTDYYQELQ RDISEMFLQI YKQGGFLGLS NIKFRPGSVV | (SEQ ID NO: 10) |
| VQLTLAFREG TINVHDVETQ FNQYKTEAAS RYNLTISDVS VSDVPFPFSA | |
| QSGAGVPGWG IALLVLVCVL VALAIVYLIA LAVCQCRRKN YGQLDIFPAR | |
| DTYHPMSEYP TYHTHGRYVP PSSTDRSPYE KVSAGNGGSS LSYTNPAVAA | |
| ASANL | |

Proopiomelanocortin (adrenocorticotropin/beta-lipotropin/alpha-melanocyte
stimulating hormone/beta-melanocyte stimulating hormone/beta-endorphin)
[Homo sapiens].
Accession number: XP_002485

| | |
|---|---|
| AAAKEGKKSR DRERPPSVPA LREQPPETEP QPAWKMPRSC CSRSGALLLA | (SEQ ID NO: 11) |
| LLLQASMEVR GWCLESSQCQ DLTTESNLLE CIRACKPDLS AETPMFPGNG | |
| DEQPLTENPR KYVMGHFRWD RFGRRNSSSS GSSGAGQKRE DVSAGEDCGP | |
| LPEGGPEPRS DGAKPGPREG KRSYSMEHFR WGKPVGKKRR PVKVYPNGAE | |
| DESAEAFPLE FKRELTGQRL REGDGPDGPA DDGAGAQADL EHSLLVAAEK | |
| KDEGPYRMEH FRWGSPPKDK RYGGFMTSEK SQTPLVTLFK NAIIKNAYKK | |
| GE | |
| RGD | |
| HHHHHHSSSSGSSSSGSSSSGGRGDSGRGDS | (SEQ ID NO: 12) |

Native Primary Sequence of the MUC1 Growth Factor Receptor (nat-PSMGFR -
An example of “PSMGFR”):

| | |
|---|---|
| GTINVHDVETQFNQYKTEAASRYNLTISDVSVSDVPFPFSAQSGA | (SEQ ID NO: 13) |

A truncated MUC1 receptor isoform having nat-PSMGFR at its N-terminus and
including the transmembrane and cytoplasmic sequences of a full-length MUC1
receptor (“nat-PSMGFRTC isoform” - An example of “PSMGFRTC” - shown
excluding optional N-terminus signal sequence - SEQ ID NOS: 19, 20, or 21
which may be cleaved after translation and prior to expression of the
receptor on the cell surface):

| | |
|---|---|
| G TINVHDVETQ FNQYKTEAAS RYNLTISDVS VSDVPFPFSA QSGAGVPGWG | (SEQ ID NO: 14) |
| IALLVLVCVL VALAIVYLIA LAVCQCRRKN YGQLDIFPAR DTYHPMSEYP | |
| TYHTHGRYVP PSSTDRSPYE KVSAGNGGSS LSYTNPAVAA ASANL | |

A truncated MUC1 receptor isoform having nat-PSMGFR and PSIBR at its
N-terminus and including the transmembrane and cytoplasmic sequences of a
full-length MUC1 receptor (“CM isoform” - shown excluding optional N-
terminus signal sequence - S SEQ ID NOS: 19, 20, or 21 which may be cleaved
after translation and prior to expression of the receptor on the cell
surface):

| | |
|---|---|
| GFLGLS NIKFRPGSVV VQLTLAFREG TINVHDVETQ FNQYKTEAAS | (SEQ ID NO: 15) |
| RYNLTISDVS VSDVPFPFSA QSGAGVPGWG IALLVLVCVL VALAIVYLIA | |
| LAVCQCRRKN YGQLDIFPAR DTYHPMSEYP TYHTHGRYVP PSSTDRSPYE | |
| KVSAGNGGSS LSYTNPAVAA ASANL | |

A truncated MUC1 receptor isoform having nat-PSMGFR + PSIBR + Unique Region
at its N-terminus and including the transmembrane and cytoplasmic sequences
of a full-length MUC1 receptor (“UR isoform” - shown excluding optional N-
terminus signal sequences SEQ ID NO: 19, 20, or 21):

| | |
|---|---|
| ATTTPASKSTPFSIPSHHSDTPTTLASHSTKTDASSTHHSTVPPLTSSNHSTSPQLSTG | (SEQ ID NO: 16) |
| VSFFFLSFHISNLQFNSSLEDPSTDYYQELQRDISEMFLQIYKQGGFLGLSNIKFRPGS | |
| VVVQLTLAFREGTINVHDVETQFNQYKTEAASRYNLTISDVSVSDVPFPFSAQSGA | |
| GVPGWGIALLVLVCVLVALAIVYLIALAVCQCRRKNYGQLDIFPARDTYHPMSEYP | |
| TYHTHGRYVPPSSTDRSPYEKVSAGNGGSSLSYTNPAVAAASANL | |

A truncated MUC1 receptor isoform including the transmembrane and cyto-
plasmic sequences of a full-length MUC1 receptor (“Y isoform” - shown
excluding optional N-terminus signal sequence - SEQ ID NOS: 19, 20, or 21
which may be cleaved after translation and prior to expression of the
receptor on the cell surface):

| | |
|---|---|
| GSGHASSTPGGEKETSATQRSSVPSSTEKNAFNSSLEDPSTDYYQELQRDISEMFLQI | (SEQ ID NO: 17) |
| YKQGGFLGLSNIKFRPGSVVVQLTLAFREGTINVHDMETQFNQYKTEAASRYNLTI | |
| SDVSVSDVPFPFSAQSGAGVPGWGIALLVLVCVLVALAIVYLIALAVCQCRRKNYG | |
| QLDIFPARDTYHPMSEYPTYHTHGRYVPPSSTDRSPYEKVSAGNGGSSLSYTNPAV | |
| AATSANL | |

A truncated MUC1 receptor isoform having nat-PSMGFR + PSIBR + Unique
Region + Repeats at its N-terminus and including the transmembrane and
cytoplasmic sequences of a full-length MUC1 receptor (“Rep isoform” - shown
excluding optional N-terminus signal sequence - SEQ ID NOS: 19, 20, or 21
which may be cleaved after translation and prior to expression of the
receptor on the cell surface):

| | |
|---|---|
| LDPRVRTSAPDTRPAPGSTAPQAHGVTS(APDTRPAPGSTAPPAHGVTS)25APDTRP | (SEQ ID NO: 18) |
| APGSTAPPAHGVTSAPDNRPALGSTAPPVHNVTSASGSASGSASTLVHNGTSARAT | |
| TTPASKSTPFSIPSHHSDTPTTLASHSTKTDASSTHHSSVPPLTSSNHSTSPQLSTGVSF | |
| FFLSFHISNLQFNSSLEDPSTDYYQELQRDISEMFLQIYKQGGFLGLSNIKFRPGSVV | |
| VQLTLAFREGTINVHDVETQFNQYKTEAASRYNLTISDVSVSDVPFPFSAQSGAGVP | |
| GWGIALLVLVCVLVALAIVYLIALAVCQCRRKNYGQLDIFPARDTYHPMSEYPTYH | |
| THGRYVPPSSTDRSPYEKVSAGNGGSSLSYTNPAVAAASANL | |

N-terminal MUC-1 signaling sequence for directing MUC1 receptor and trun-
cated isoforms to cell membrane surface (optionally present, in whole or
part - e.g. up to 3 a.a. may be absent at C-terminal end as indicated by
variants in SEQ ID NOS: 19, 20, and 21, at N-terminus of above-listed MUC1
truncated receptor isoforms):

| | |
|---|---|
| MTPGTQSPFFLLLLLTVLT. | (SEQ ID NO: 19) |
| MTPGTQSPFFLLLLLTVLT VVTA | (SEQ ID NO: 20) |
| MTPGTQSPFFLLLLLTVLT VVTG | (SEQ ID NO: 21) |

An example of a Histidine-Tagged Primary Sequence of the MUC1 Growth Factor
Receptor (His-var-PSMGFR) (having “SPY” sequence of var-PSMGFR) having a
single amino acid deletion at the C-terminus of SEQ ID NO: 2):

| | |
|---|---|
| TINVHDVETQFNQYKTEAASPYNLTISDVSVSDVPFPFSAQSGAHHHHHH | (SEQ ID NO: 22) |

Histidine-Tagged Tumor-Specific Extended Sequence of MUC1 Growth Factor
Receptor (TSESMGFR) (having “SPY” sequence of var-PSMGFR):

| | |
|---|---|
| SVVVQLTLAFREGTINVHDVETQFNQYKTEAASPYNLTISDVSVSDVPFPFSAQSGA | (SEQ ID NO: 23) |
| HHHHHH | |

Histidine-Tagged Truncated Interchain binding Region (His-TPSIBR):

| | |
|---|---|
| HHHHHHSVVVQLTLAFREG | (SEQ ID NO: 24) |

Native Primary Sequence of the MUC1 Growth Factor Receptor (nat-PSMGFR - An
example of “PSMGFR”), having a single amino acid deletion at the C-terminus
of SEQ ID NO: 13):

| | |
|---|---|
| TINVHDVETQFNQYKTEAASRYNLTISDVSVSDVPFPFSAQSGA | (SEQ ID NO: 25) |

“SPY” functional variant of the native Primary Sequence of the MUC1 Growth
Factor Receptor having enhanced stability (var-PSMGFR - An example of
“PSMGFR”), having a single amino acid deletion at the C-terminus of
SEQ ID NO: 7):

| | |
|---|---|
| TINVHDVETQFNQYKTEAASPYNLTISDVSVSDVPFPFSAQSGA | (SEQ ID NO: 26) |
| Truncated Interchain Binding Region) (TPSIBR): | |
| SVVVQLTLAFREG | (SEQ ID NO: 27) |

Tumor-Specific Extended Sequence of MUC1 Growth Factor Receptor (TSESMGFR)
(having “SPY” sequence of var-PSMGFR):

| | |
|---|---|
| SVVVQLTLAFREGTINVHDVETQFNQYKTEAASPYNLTISDVSVSDVPFPFSAQSGA | (SEQ ID NO: 28) |
| H₂N-GTINV HDVET QFNQY KTEAA SRYNL TISDV SVSDV PFPFS AQSGA | (SEQ ID NO: 29) |
| HHHHHH-CO₂H | |

TABLE 2
compounds 1-51 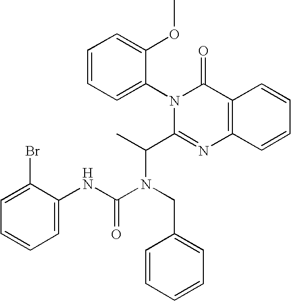
1 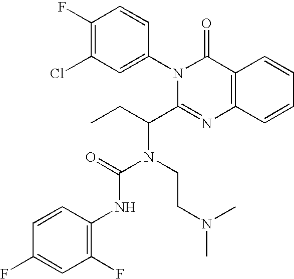
2 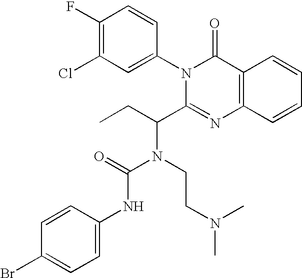
3 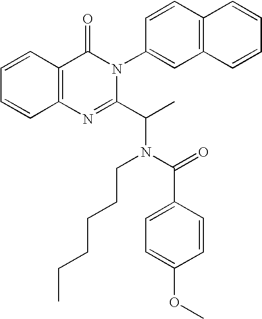
4 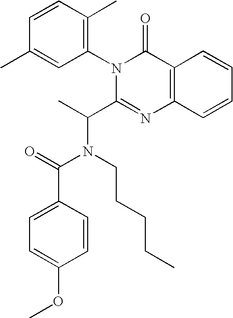
5 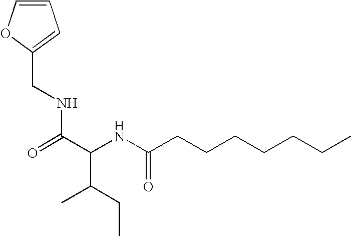
6 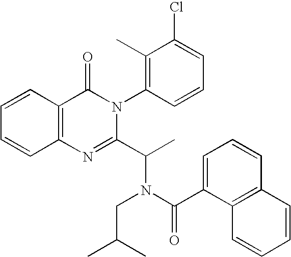
7 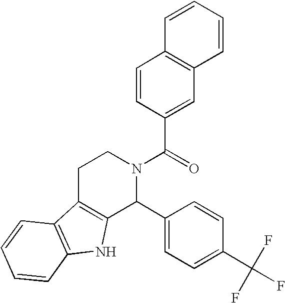
8 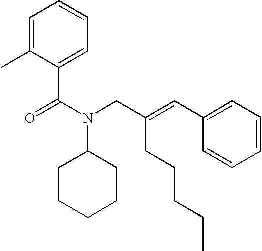
9 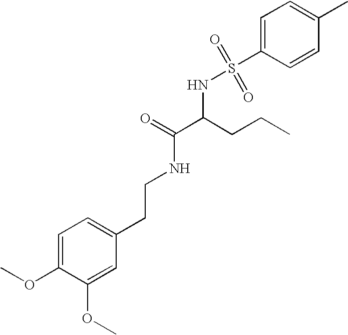
10 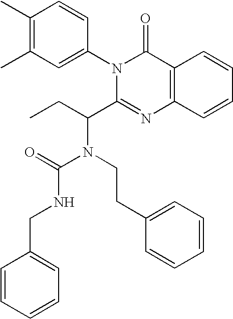
11 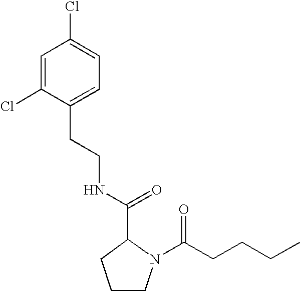
12 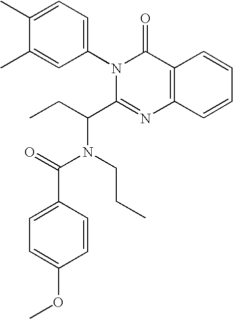
13 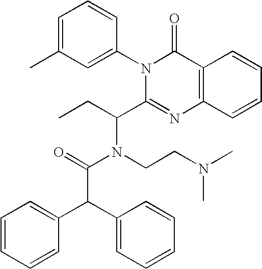
14 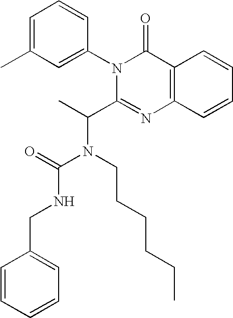
15 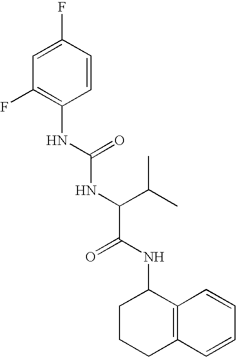
16 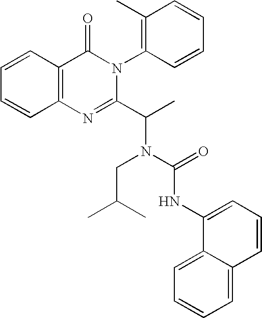
17 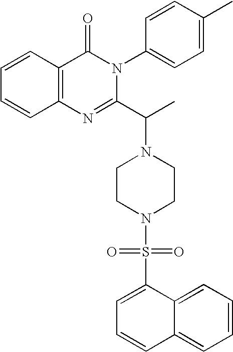
18 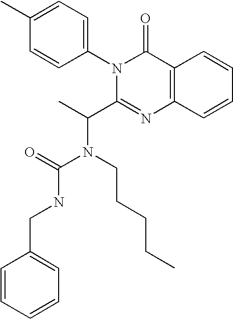
19 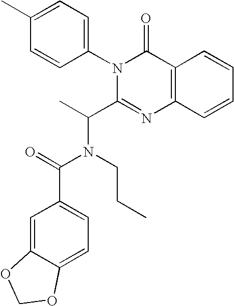
20 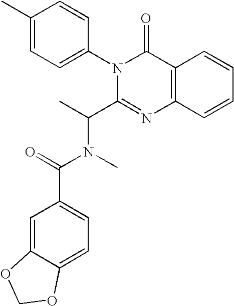
21 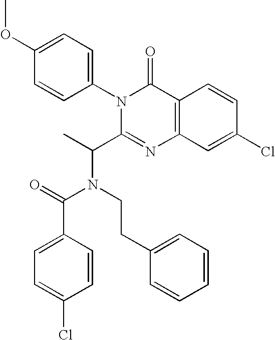
22 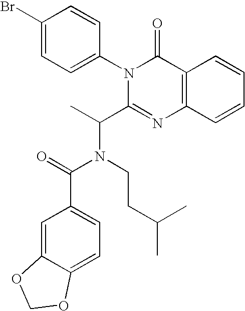
23 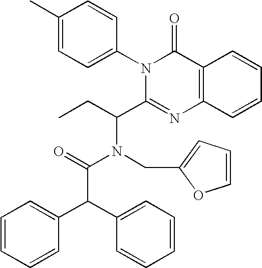
24 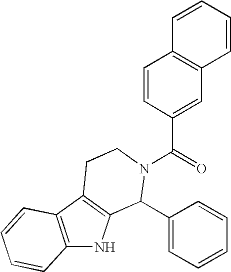
25 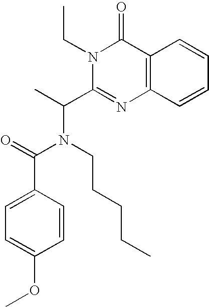
26 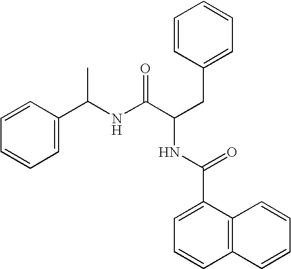
27 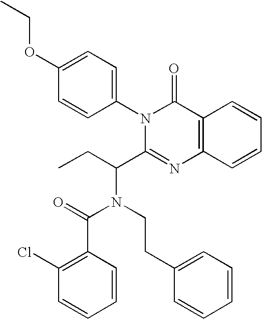
28 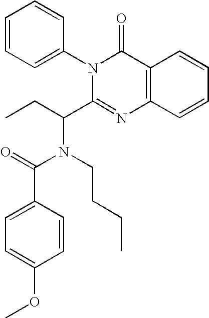
29 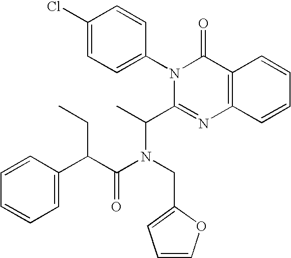
30 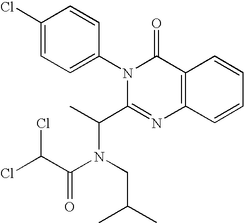
31 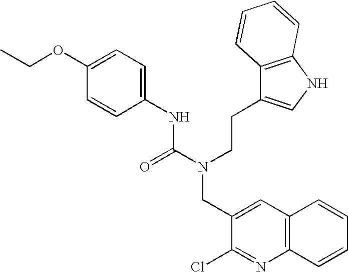
32 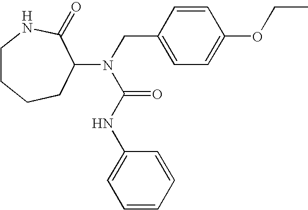
33 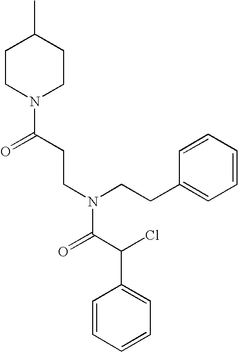
34 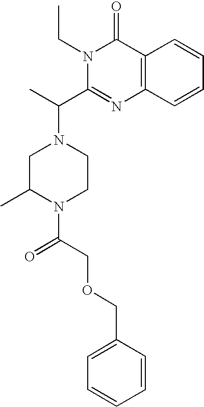
35 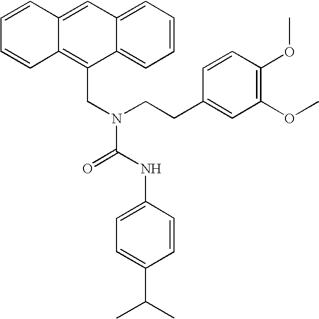
36 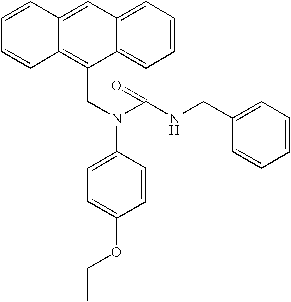
37 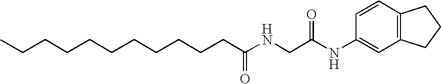
38 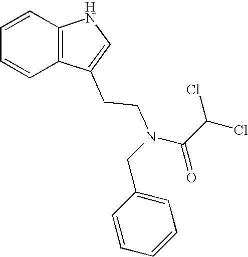
39 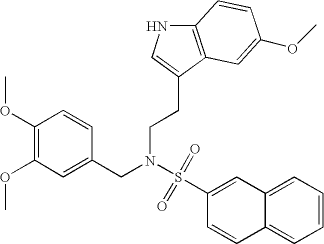
40 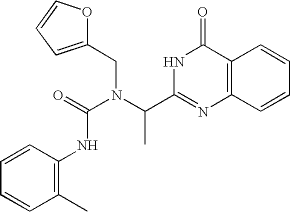
41 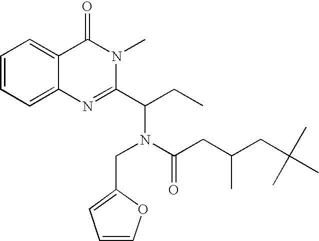
42 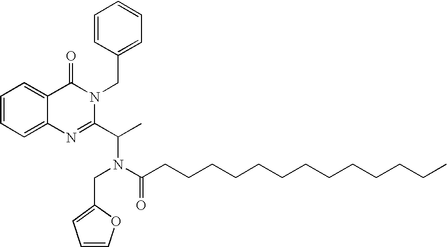
43 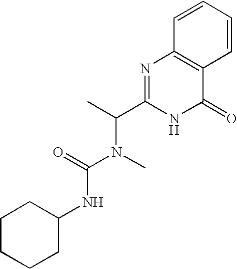
44 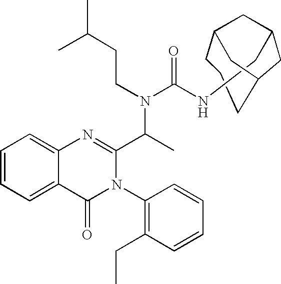
45 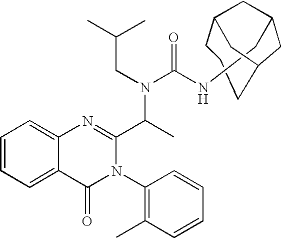
46 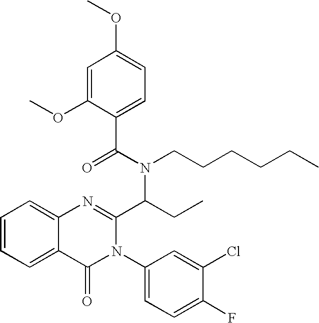
47 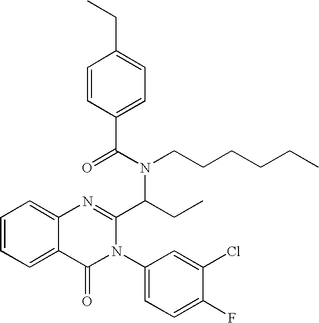
48 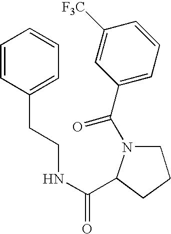
49 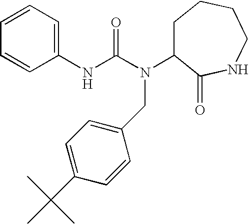
50 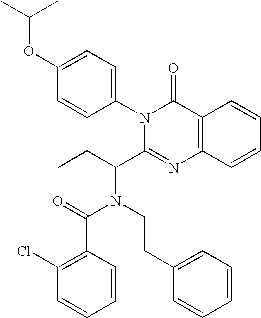
51

TABLE 3
COMPOUNDS 52-106 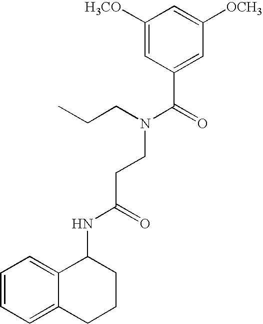
52 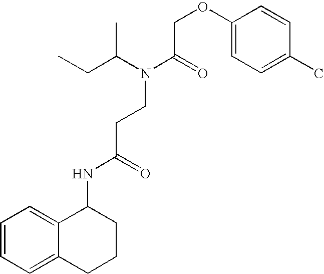
53

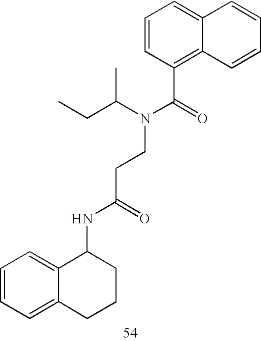

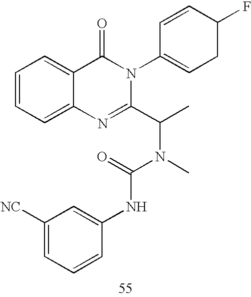

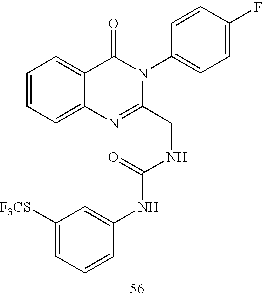

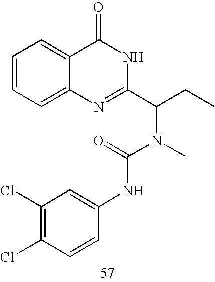

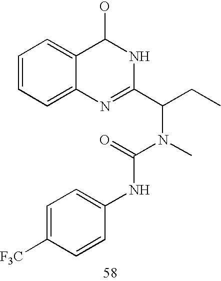

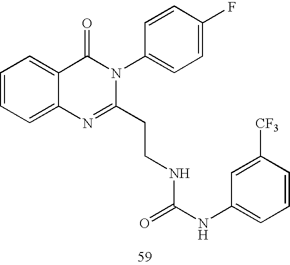

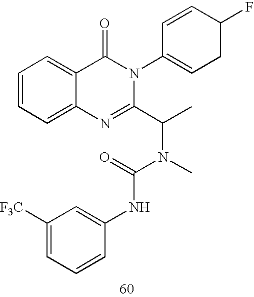

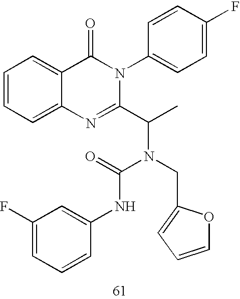

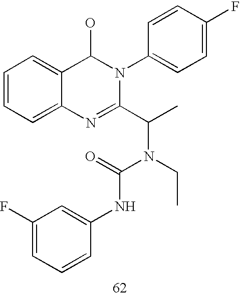

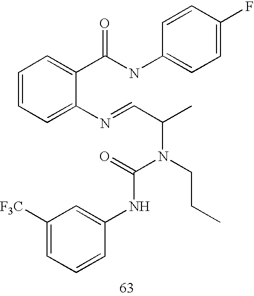

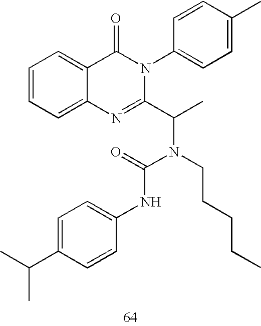

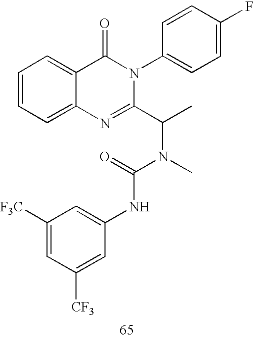

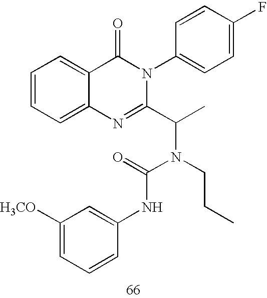

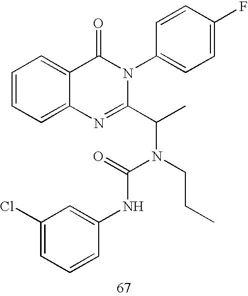

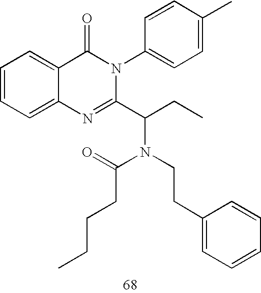

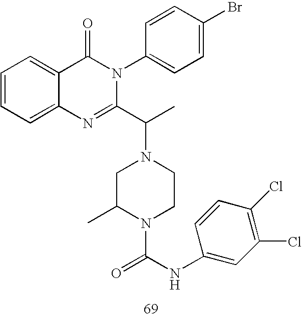

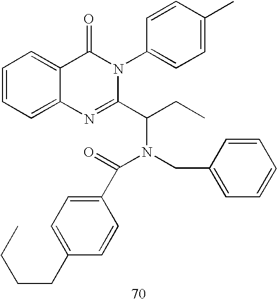

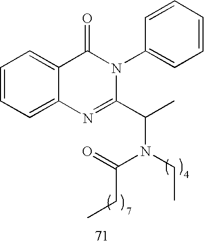

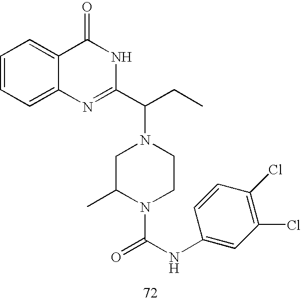

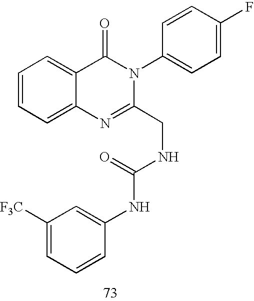

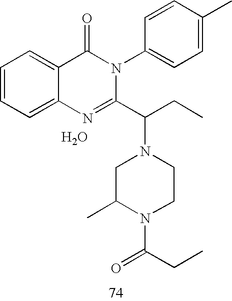

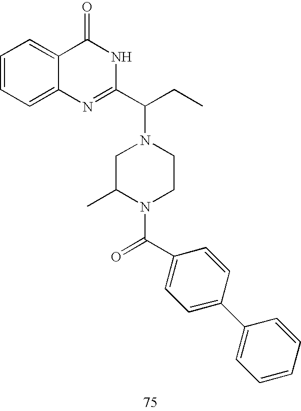

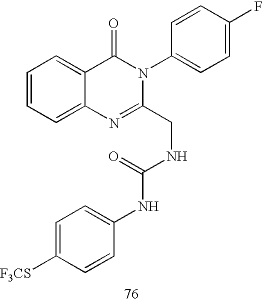

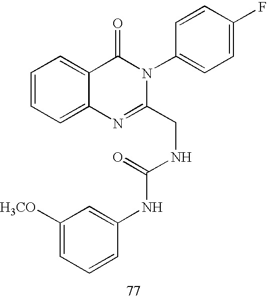

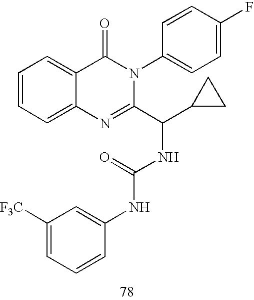

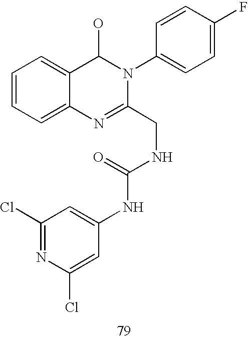

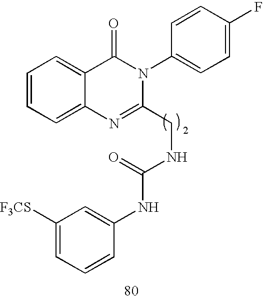

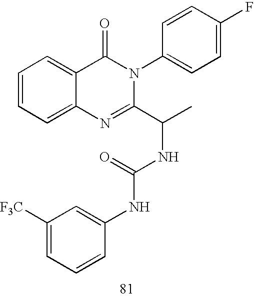

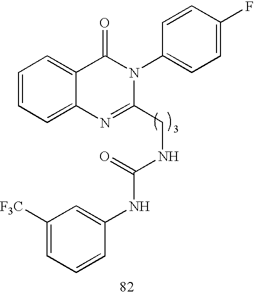

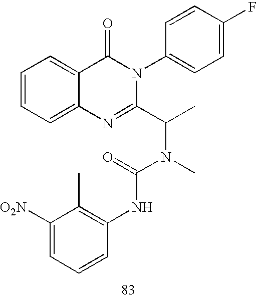

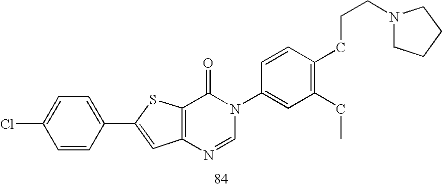

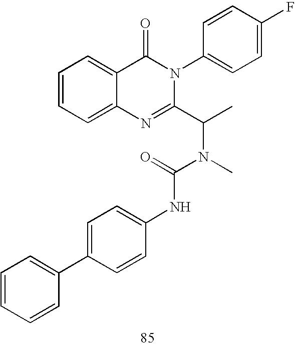

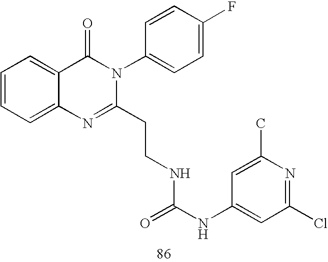

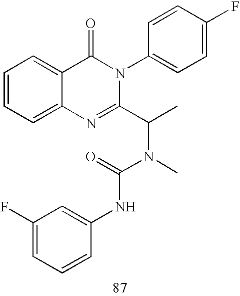

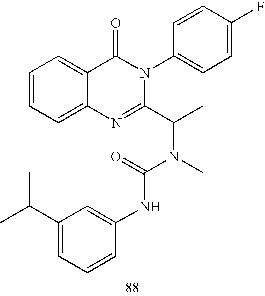

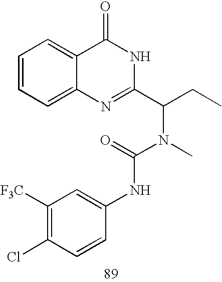

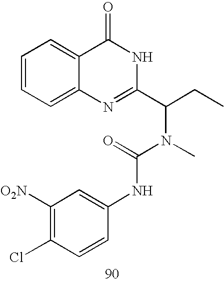

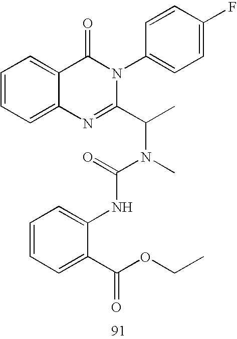

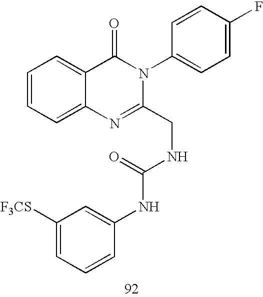

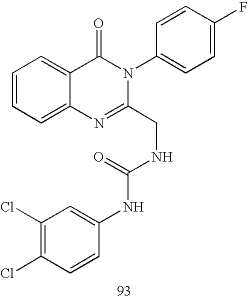

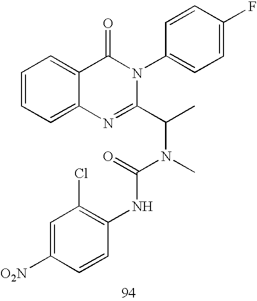

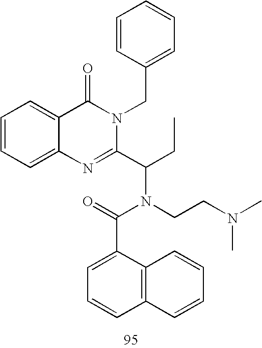

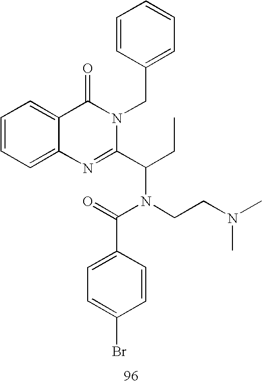

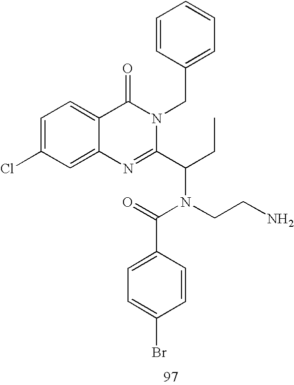

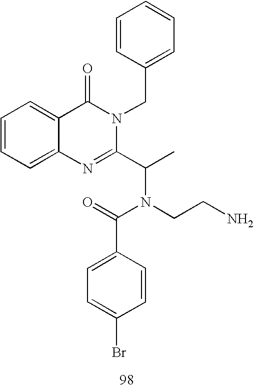

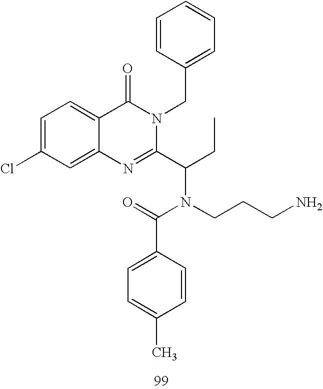

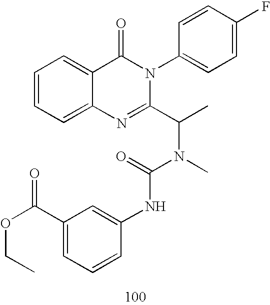

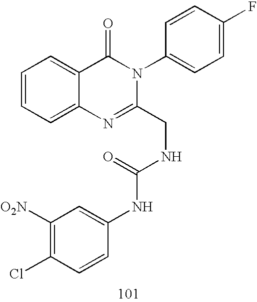

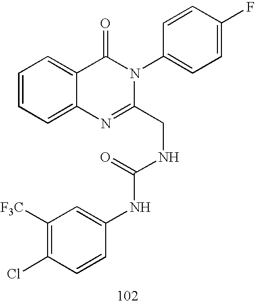

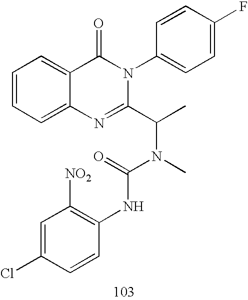

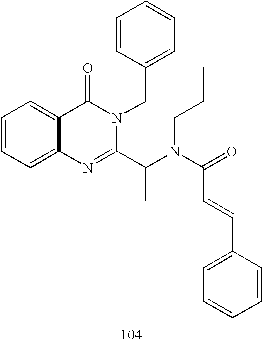

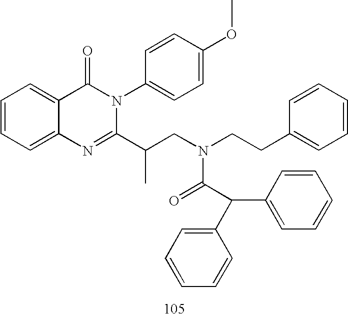

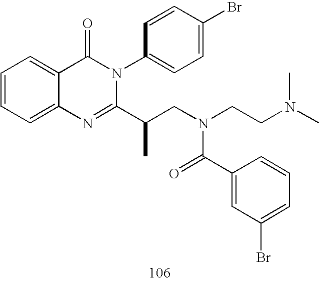

TABLE 4
COMPOUNDS 107-173

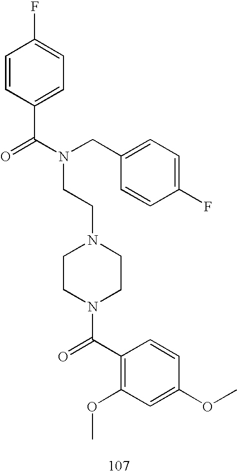

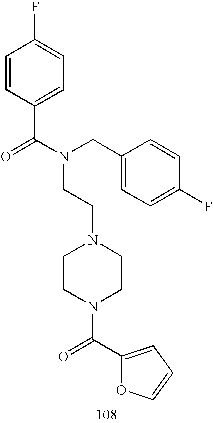

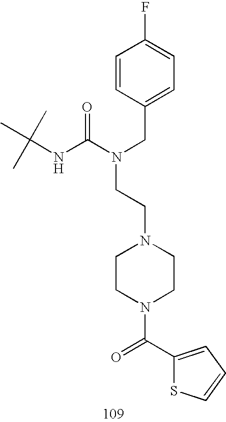

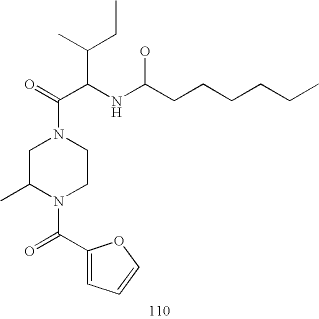

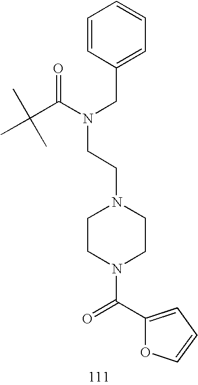

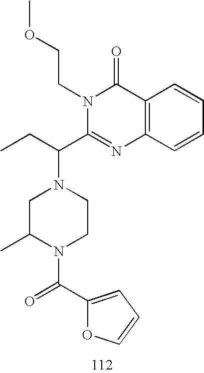

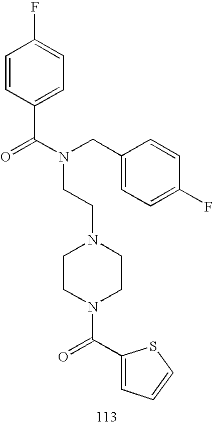

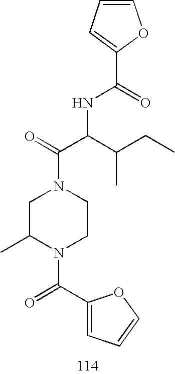

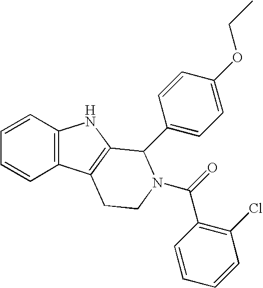

115

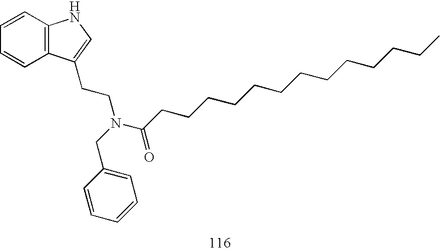

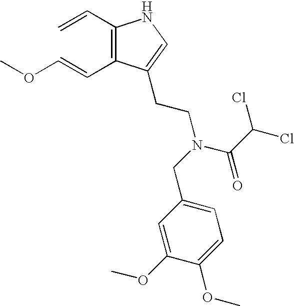

117

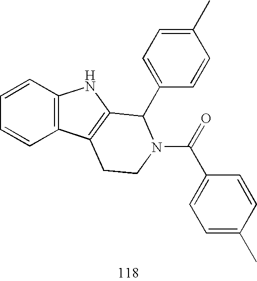

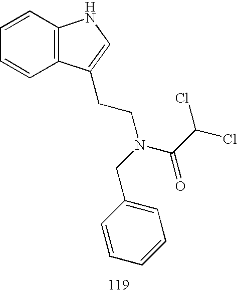

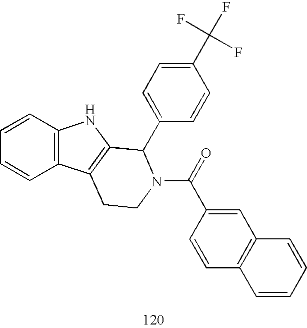

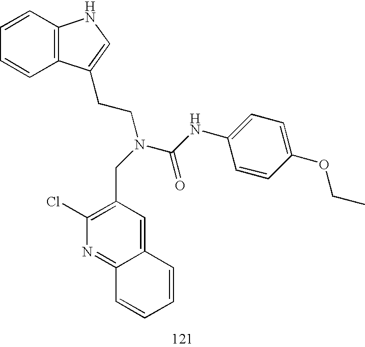

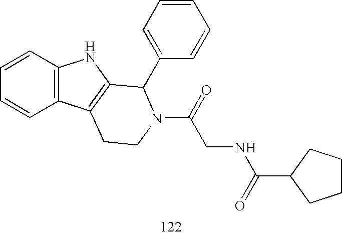

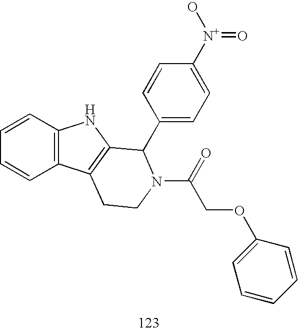

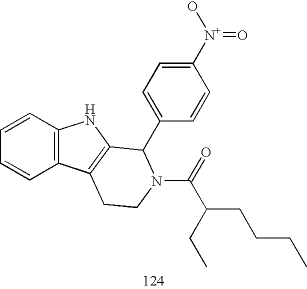

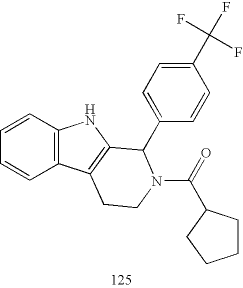

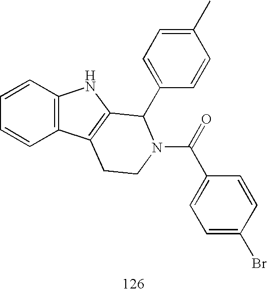

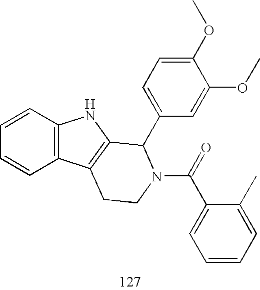

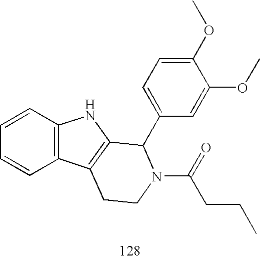

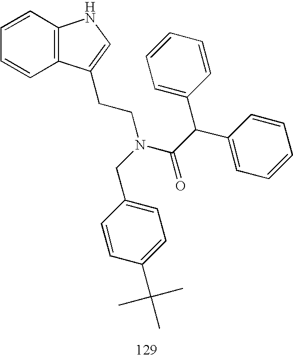

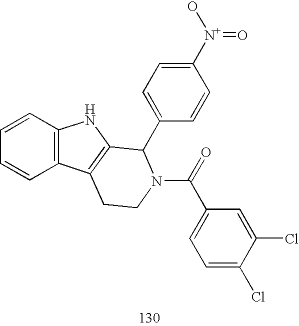

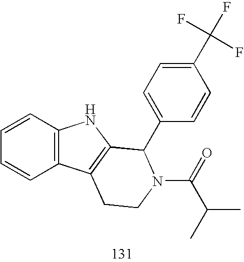

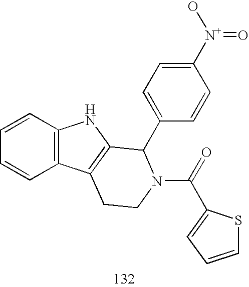

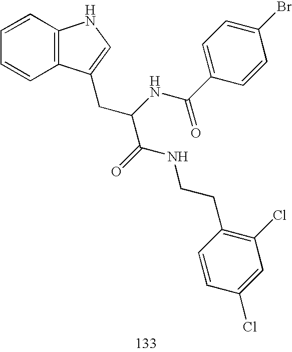

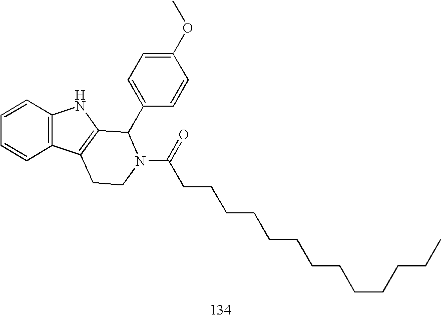

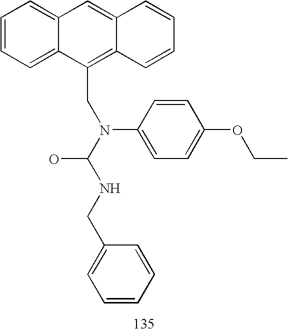

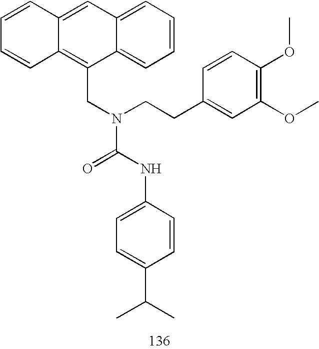

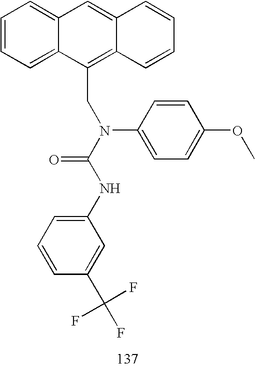

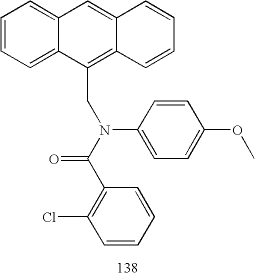

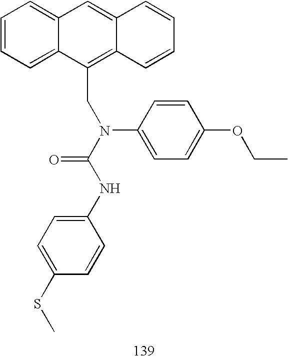

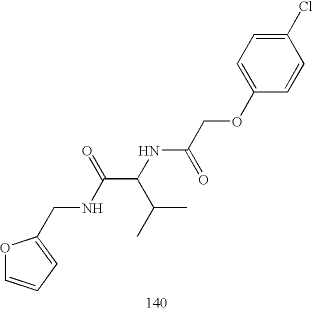

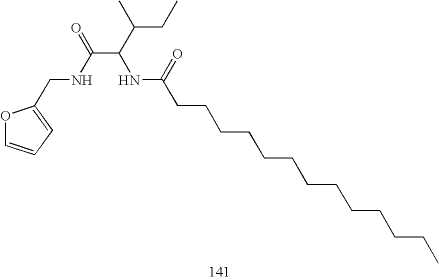

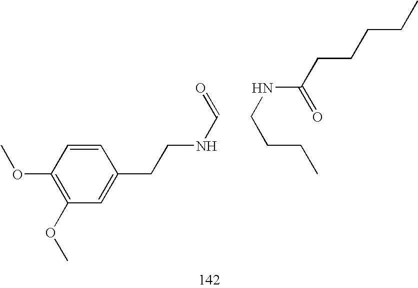

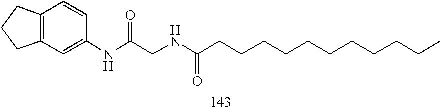

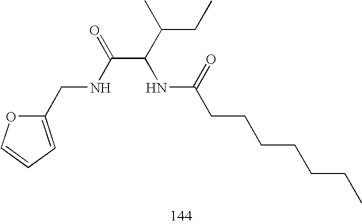

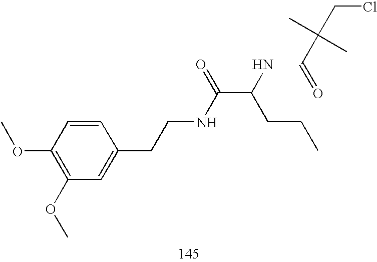

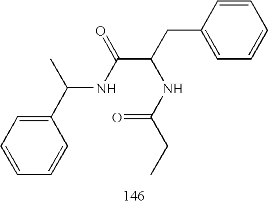

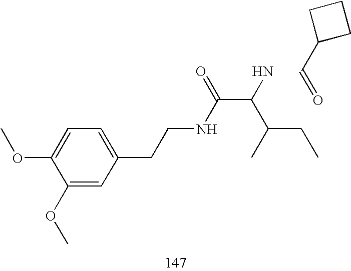

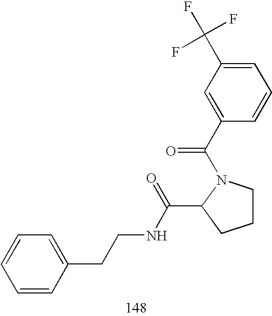

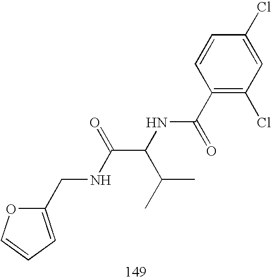

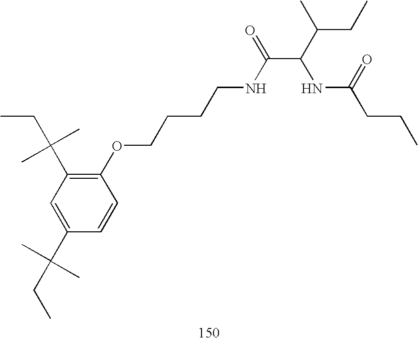

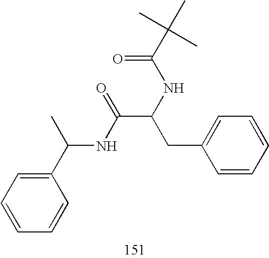

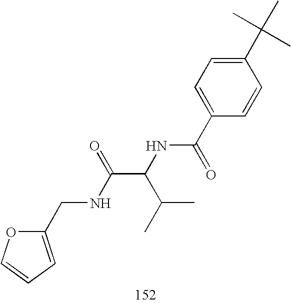

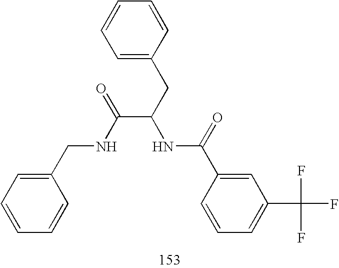

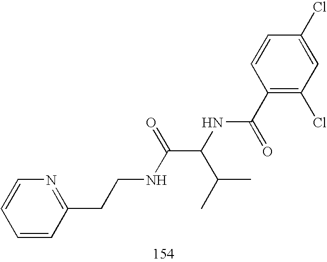

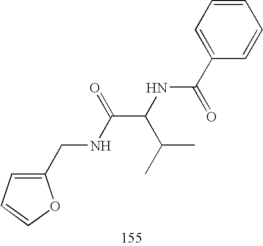

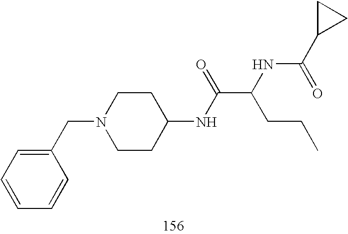

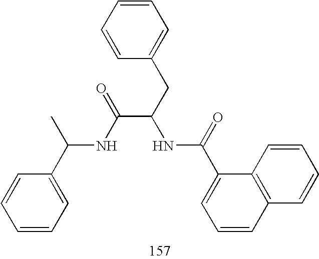

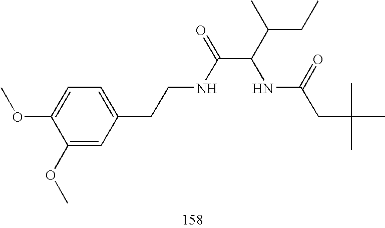

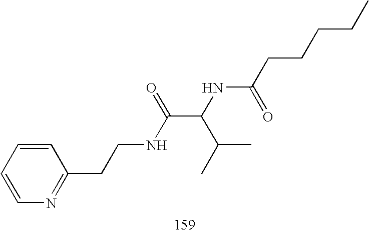

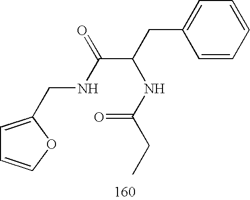

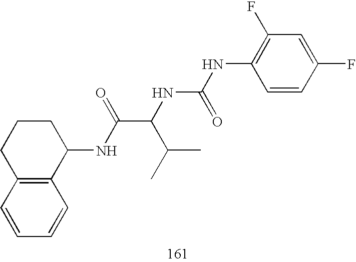

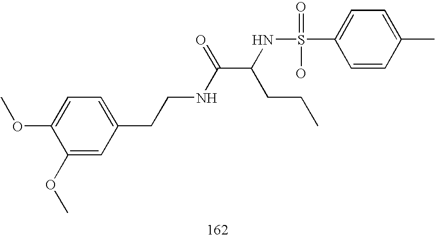

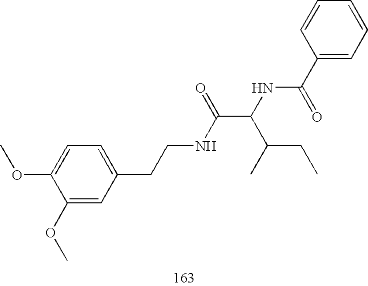

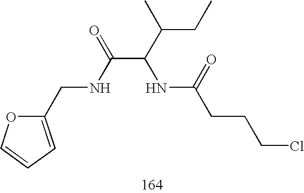

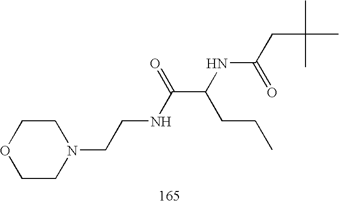

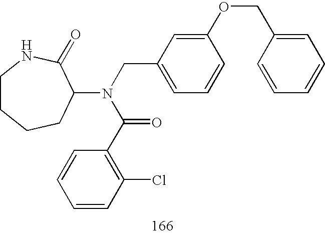

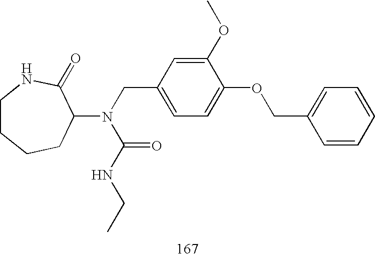

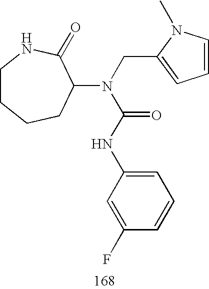

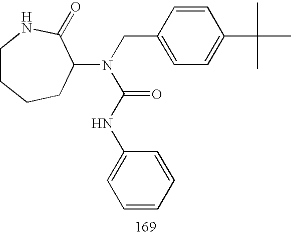

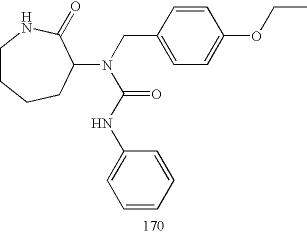

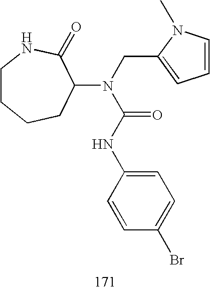

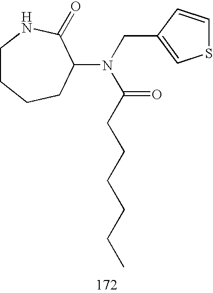

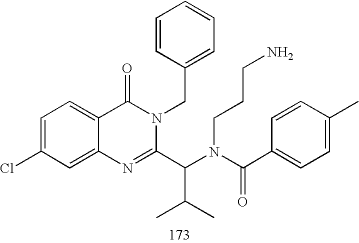

TABLE 5
COMPOUNDS 174-188

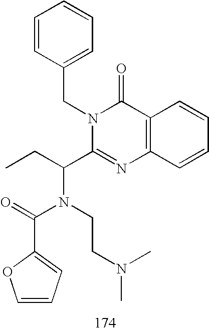

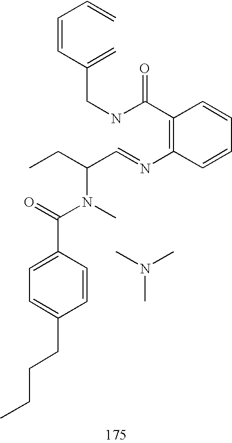

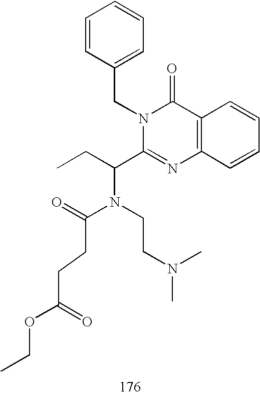

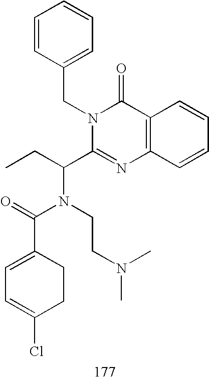

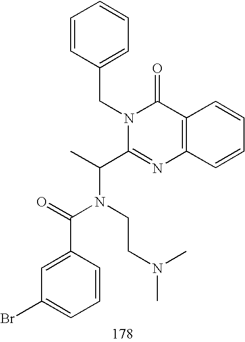

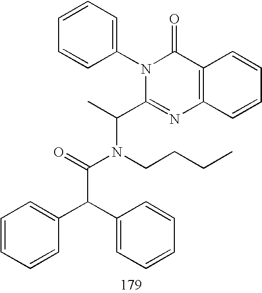

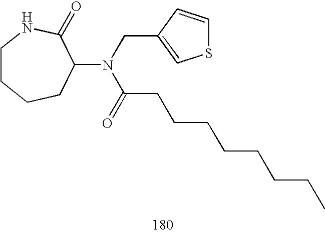

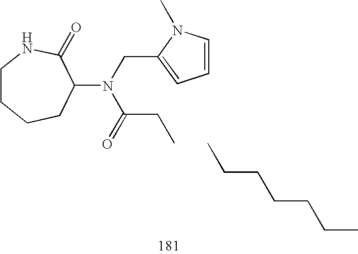

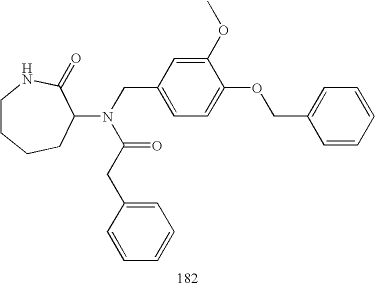

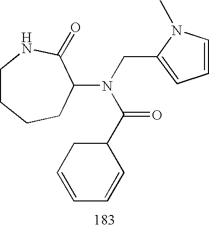

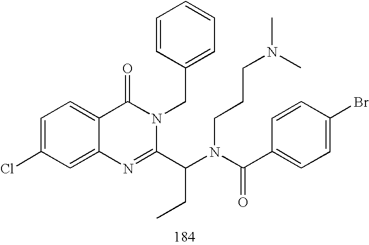

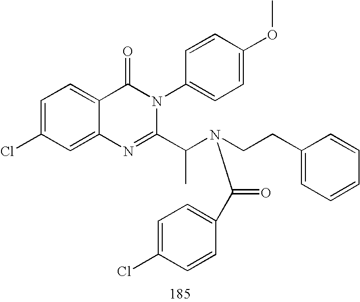

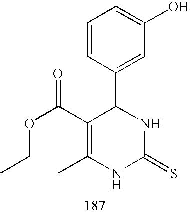

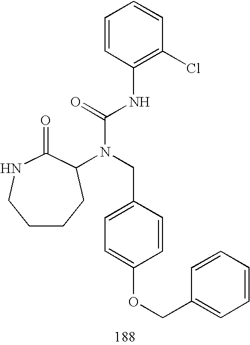

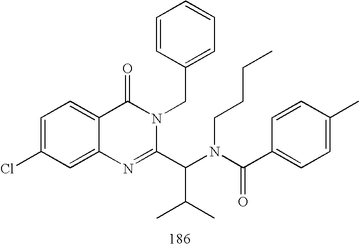

The following examples are intended to illustrate certain aspects and embodiments of the present invention, but do not exemplify the full scope of the invention.

### EXAMPLES

### Example 1

A tumor from a patient suspected of having cancer is biopsied. The tissue specimen is assessed using the standard methods known to those skilled in the art to enable an assessment of whether or not the specimen is cancerous. The tissue specimen is then characterized as to whether it is a MUC1-positive or negative cancer using methods of the invention. The patient is diagnosed as having a MUC1-positive cancer. The physician prescribes a treatment that includes an agent(s) that inhibits MUC1. Preferred treatments are with agents that bind to the MGFR portion of the MUC1 receptor and/or inhibit its cleavage. Especially preferred are compounds disclosed in Tables 2-5, and in particular, compound numbers 28, 118, 107, 109, 125, 173, 182, 184, 185, and 188.

### Example 2

### Antibody Production

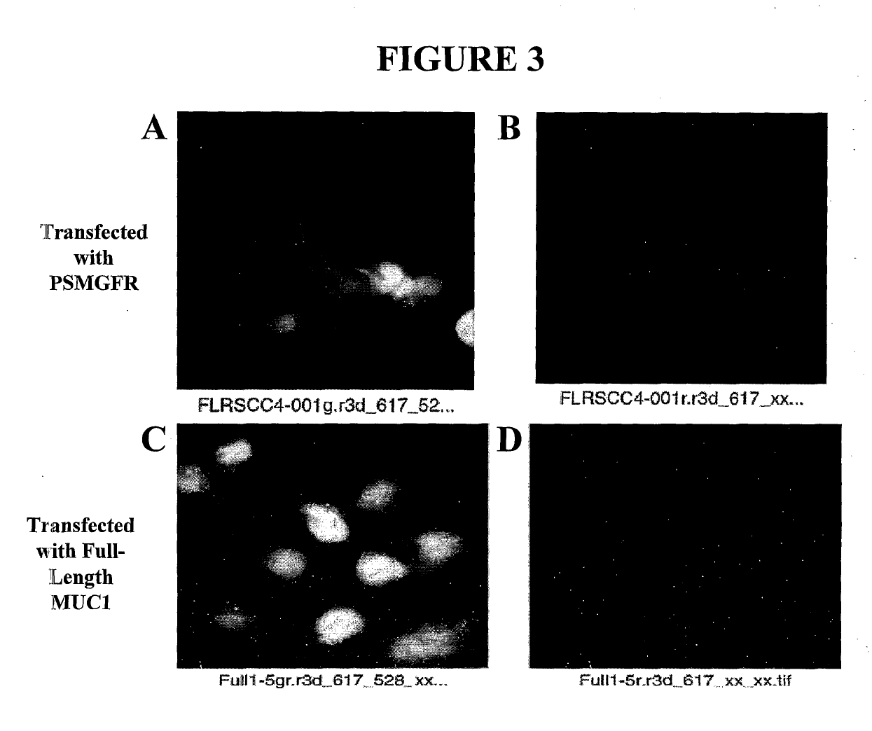

This example demonstrates that antibodies raised against the MGFR region of the MUC1 receptor, specifically bind to the form of the MUC1 receptor that is predominantly expressed on cancer cells. In this example it is shown that inventive antibodies raised against the MGFR bind to a MUC1 species that is expressed on cancer cells, wherein virtually all of the tandem repeat units have been cleaved and shed from the cell surface leaving the MGFR attached to the cell surface. Inventive antibodies were raised against the PSMGFR portion of the MUC1 receptor, in particular nat-PSMGFR or var-PSMGFR shown in Table 1 using standard methods of antibody production. Rabbit polyclonal antibodies were produced and purified by column chromatography in which the immunizing peptide was attached to the chromatography column beads. The antibodies, anti-nat-PSMGFR and anti-var-PSMGFR, were shown to specifically and sensitively bind to the immunizing peptide, MUC1 extracted from tumor cells (See FIGS. 1 and 2), MUC1 on the surface of tumor cells, (See FIG. 3), and MUC1 presented on tumor cells in intact tissue specimens, (See FIGS. 4-10).

### Example 3

### MUC1-Positive Breast Cancers

Formalin fixed, paraffin embedded anonymous tissue specimens were separately tested for reactivity to two antibodies that recognize different epitopes on the MUC1 receptor: 1) a rabbit polyclonal antibody, anti-PSMGFR, that binds to the PSMGFR portion of the MUC1 receptor that remains attached to the cell surface after receptor shedding; and 2) a commercially available mouse monoclonal, VU4H5 (Santa Cruz) that binds to a sequence in the tandem repeat section of the receptor. Both antibodies were at 1 ug/ml. Protocols and treatments that the specimens were subjected to were identical for both antibodies. Contiguous sections from the same tissue specimen block were separately probed with either VU4H5 or anti-PSMGFR so that nearly identical sections were compared. As controls, like specimens were also probed with irrelevant mouse and rabbit antibodies. One section from each block was stained with hemotoxin and eosin (H&E) to aid in assessing tumor grade. Specimens were processed using Dako reagents and kits for IHC, which included Dako's antigen retrieval protocol using TRS-Low pH, a low (6.0) pH citrate buffer.

Tissue specimens were visually analyzed following tissue staining. It was observed that anti-PSMGFR was superior as a staining reagent for the specimens in terms of sensitivity and specificity. In addition, anti-PSMGFR produced clear membrane staining whereas VU4H5 produced diffused cytoplasmic staining that was often non-existent in the most cancerous specimens or the most cancerous regions of a specimen.

### Example 3.1

### Results

### Example 3.1.1

### Breast cancer

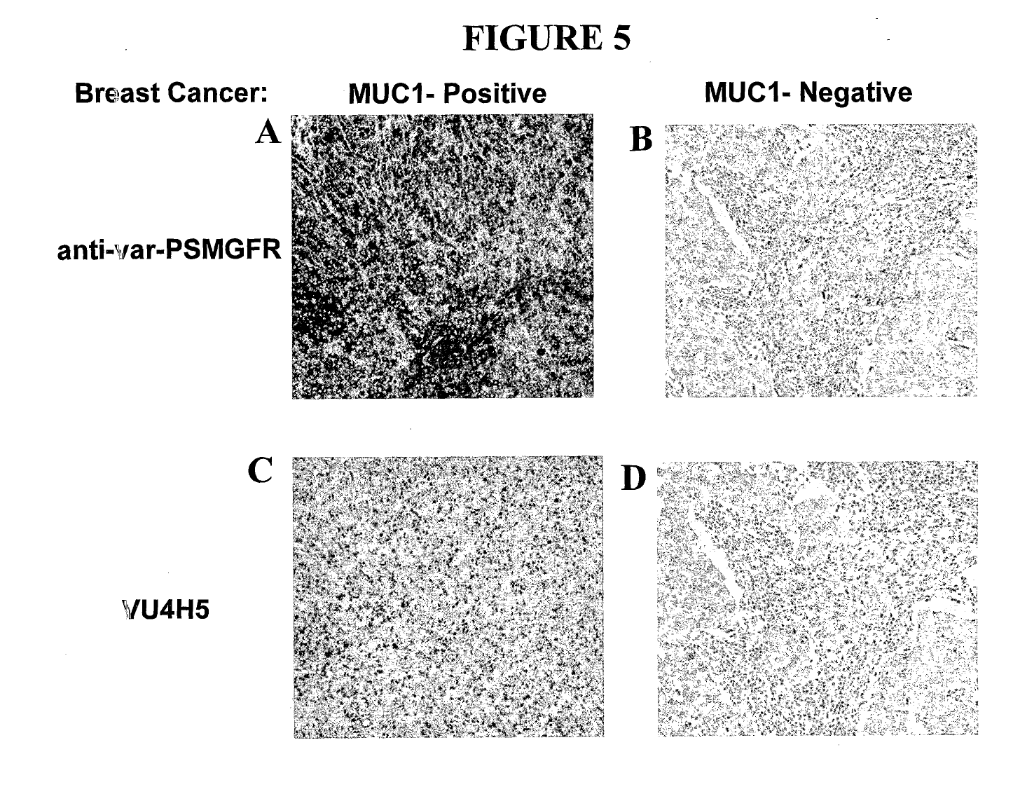

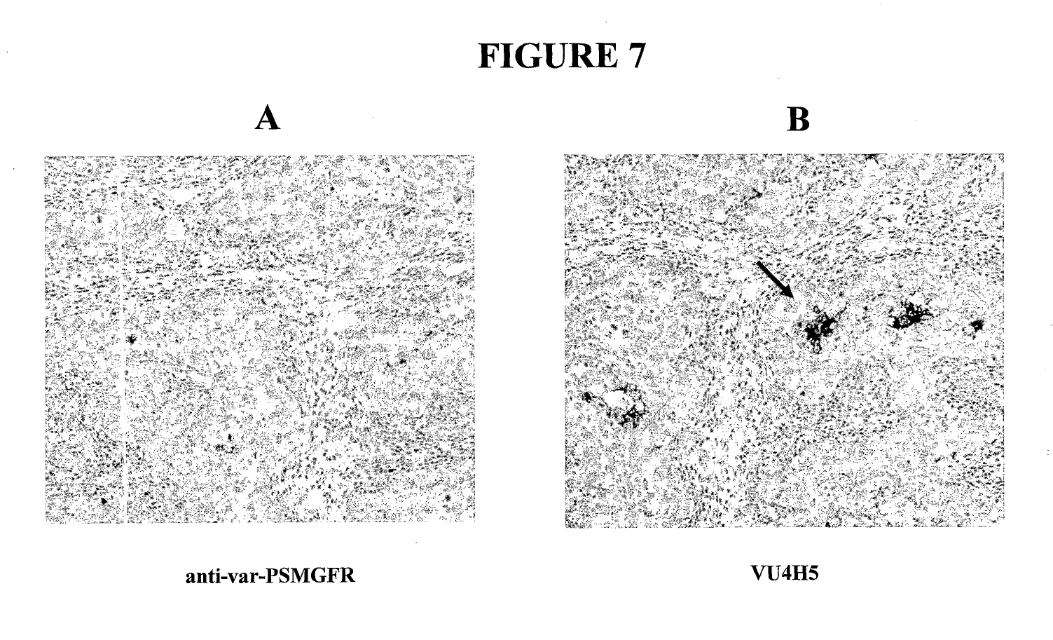

Breast tissue specimens that were normal, cancerous and benign were analyzed. Anti-PSMGFR intensely stained all the cancerous regions of the specimens. Staining was membrane specific and showed uniform distribution of the receptor over the cell surface. The staining formed a chicken wire pattern, (See FIGS. 4-5). Ducts in cancerous regions were intensely stained and staining was not limited to the apical border but was spread over the entire cell surface. 4-5 layers of cells surrounding the duct stained positive and displayed uniform membrane staining. “Normal” ducts within this cancerous specimen stained positive at the apical border with anti-PSMGFR but not VU4H5. VU4H5 also stained cancerous breast tissue. However, the staining was much less intense; staining was diffuse and cytoplasmic (See FIG. 5). Importantly, some of the regions that were the most cancerous and stained the most intensely with anti-PSMGFR did not stain positive with VU4H5. This is consistent with the idea that in the most cancerous regions, virtually all the receptors have been cleaved to the growth factor receptor form, which consists essentially of PSMGFR. Two specimens were cancerous but MUC1-negative. Neither of these MUC1-negative specimens stained positive with anti-PSMGFR, although they did present the MUC1 receptor, but presumably at normal levels and in their normal cleavage state. However, some VU4H5 staining was observed in necrotic areas of the sections (See FIG. 7). This could give rise to false positives if VU4H5 were to be used as the diagnostic reagent.

A suitable diagnostic protocol for visual determination of which breast cancers are MUC1-positive or negative is as follows. MUC1-positive cancers: Specimen stains positive with anti-PSMGFR in regions away from normal healthy ducts. Staining is membrane specific and uniformly distributed over entire cell surface. Involved regions produce chicken wire pattern staining. MUC1-negative cancers: Specimen shows no staining in regions away from ducts. Staining with anti-PSMGFR is limited to membrane staining focused at the apical border of ducts.

### Example 3.1.2

### Lung cancer

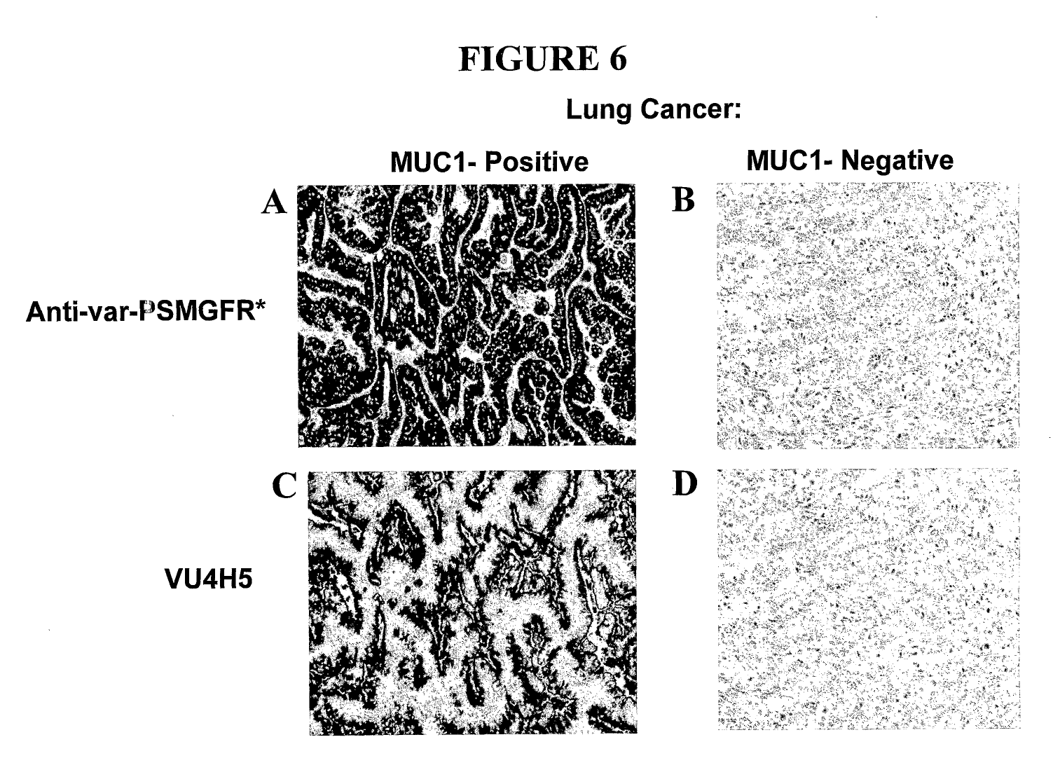

Lung tumor specimens were analyzed. MUC1-positive cancer specimen staining with anti-PSMGFR was very similar to the staining observed for the breast cancer specimens described above. Anti-PSMGFR produced intense membrane staining wherein staining was uniformly distributed over cell surface. Staining was uniform in cancerous regions of the specimen (See FIGS. 4 and 6). Staining with VU4H5 was sufficient to discriminate MUC1-positive cancers from MUC1-negative cancers for the specimens analyzed, but the staining was diffuse and cytoplasmic (See FIG. 6). MUC1-negative cancer specimens were tissue specimens that were clearly identifiable as cancerous specimens but did not stain with either anti-PSMGFR or VU4H5 (See FIG. 6).

A suitable diagnostic protocol for visual determination of which lung cancers are MUC1-positive or negative is as follows. MUC1-positive cancers: MUC1-positive cancer: specimen stains positive with anti-PSMGFR in contiguous regions. Membrane staining that is uniformly distributed over cell surface is produced. Involved regions produce chicken wire-like staining pattern. MUC1-negative cancer: no staining with either anti-PSMGFR or VU4H5.

### Example 3.1.3

### Colon cancer

Colon cancer specimens and normal colon specimens were analyzed. Anti-PSMGFR produced very intense staining of cancerous regions of the specimens. Large areas of the specimen were stained black (See FIG. 9). Areas in which cells were identifiable displayed membrane staining wherein the stain was uniformly distributed over the entire cell surface. VU4H5 stained some of the regions that were stained by anti-PSMGFR although most of these cancerous areas did not stain with this antibody that binds to the portion that is typically shed from tumor cells. The ducts of normal colon tissue and the normal ducts within the cancer specimens stained positive with anti-PSMGFR. This staining showed clustered receptors lining the luminal side of the ducts, i.e. normal apical staining. VU4H5 staining was mostly diffuse and cytoplasmic in these normal cells lining the ducts.

A suitable diagnostic protocol for visual determination of which colorectal cancers are MUC1-positive or negative is as follows. MUC1-positive cancers: Specimen stains positive with anti-PSMGFR in regions other than ducts. Staining is membrane specific and uniformly distributed over cell surface. Large regions of stained cells and debris are observed. MUC1-negative: No staining in regions away from ducts.

### Example 3.1.4

### Prostate Cancer

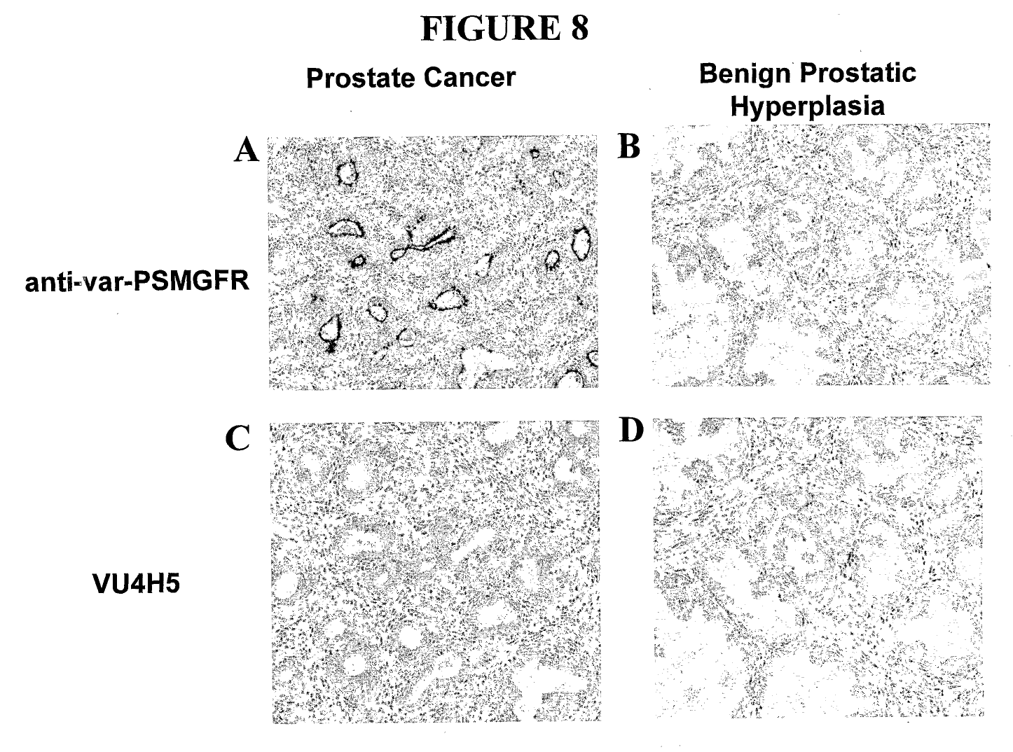

Prostate cancer specimens as well as benign prostatic hyperplasia specimens were analyzed. Cancerous prostate specimens were characterized by apical staining of the ducts with anti-PSMGFR but not with VU4H5 (See FIG. 8) whereas normal prostate specimens stained positive with both antibodies. It appears from these samples that the scoring protocol for determining which prostate cancers are MUC1-positive or negative must include the ratio of anti-PSMGFR staining to staining using an antibody that recognizes portions of the MUC1 receptor that are typically shed from tumor cells, such as VU4H5. A high ratio of anti-PSMGFR to VU4H5 would be indicative of a MUC1 positive cancer.

### Example 4

### Predicting the Potential of a Benign Breast Tumor to Develop Into a Cancer.

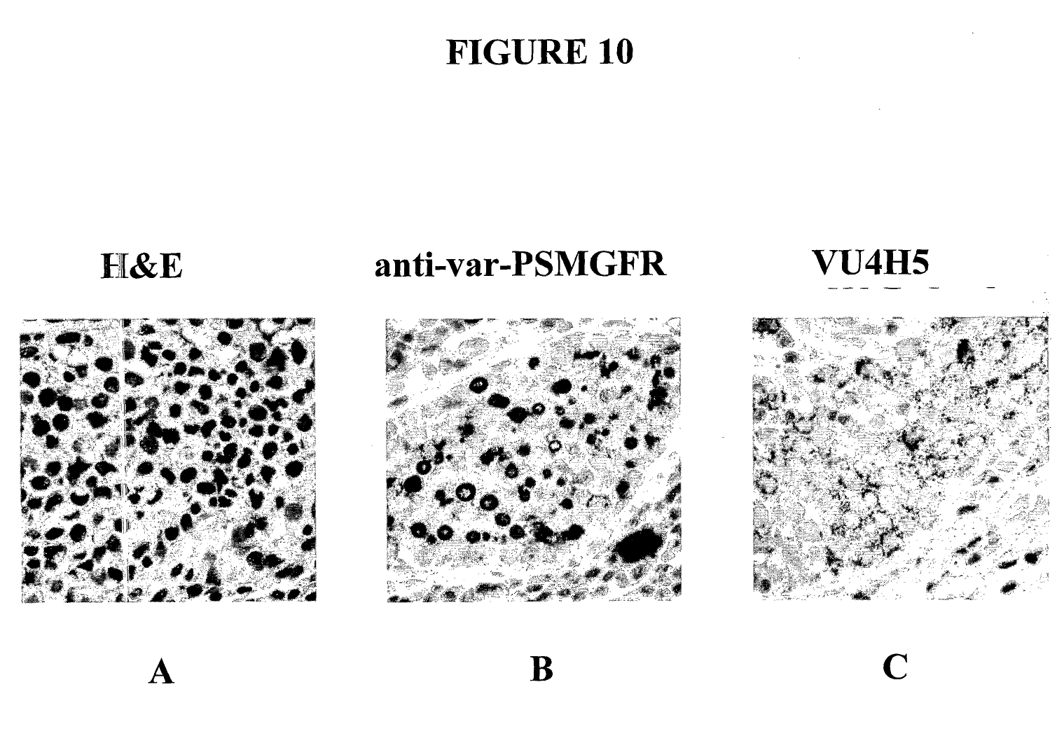

Many tumor tissue specimens contain regions of “normal” cells, displastic cells, and cancerous cells. The spatial transition from normal, to displastic, to cancerous can be correlated to the temporal transition of a developing cancer. It was observed that the displastic cells of MUC1-positive cancer specimens were stained with anti-var-PSMGFR in a manner that was distinct from staining of displastic cells from non-cancerous specimens or from MUC1-negative cancer specimens. Anti-var-PSMGFR stained a clear ring inside the cells (See FIG. 10); the more displastic the cell, the bigger the ring. One could trace the transition from normal to displastic to cancerous within a specimen as anti-PSMGFR staining went from zero to intracellular rings to uniform membrane staining. This internal ring staining was observed in every MUC1-positive breast cancer specimen tested in this study. Currently, women with tumors that are scored benign but displastic are typically issued “wait & see” advice. Results disclosed herein indicate that intracellular ring staining with anti-var-PSMGFR predicts that those benign but displastic tumors will evolve into cancers. Therefore, patients diagnosed with benign but displastic tumors that produce intracellular ring staining with anti-var-PSMGFR should be treatment with anti-cancer agents, preferentially agents that directly or indirectly inhibit MUC1, even more preferred are agents that inhibit the PSMGFR region of MUC1 and still more preferred are articles of the invention that inhibit MUC1.

### Example 5

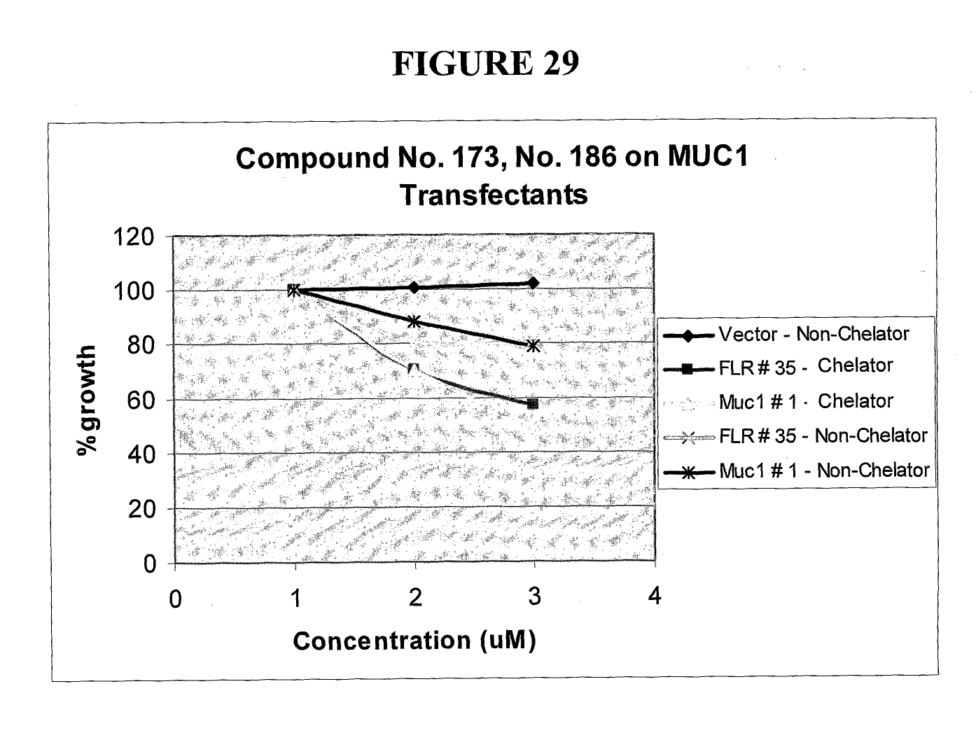

This experiment was conducted and showed that compounds that have a metal chelate functionality and bind to the MGFR region of MUC1 inhibit MUC1 cleavage. In particular, the experiments described herein show that Compounds Nos. 173, 28, and 184 inhibit the cleavage of MUC1 on live, growing cells. The design of the experiment was as follows: Breast tumor cells T47D and ZR-75-1 are grown under normal conditions except that an agent, PMA, known to increase the cleavage of MUC1, is added to the cells. In addition, compounds thought to bind to PSMGFR region of MUC1 and also possess a metal chelate function that disables MMPs thought to cleave MUC1, are individually added. If the test compounds inhibit cleavage of MUC1, then the conditioned media from around the cells have a decreased amount of shed MUC1. The amounts are visualized by western blot. Results show that Compounds Nos. 173, 28, and 184 all inhibit the cleavage of MUC1. See FIG. 29.

### Example 5.1

### Experimental Protocol

Approximately 800,000 cells (T47D or ZR-75-1) were plated per well into the wells of a 24 well plate. Cells were allowed to attach overnight and the media changed to serum free media the next day. Just prior to adding PMA (Phorbol-12-mmyristate-13-acetate, Calbiochem, La Jolla), with or without compounds/inhibitors, the media was changed once again with 1 ml of serum free media. The reagents (PMA, inhibitor) were added and the cells were allowed to incubate with the reagents for one hour. The cells were observed under the microscope for survival and morphology. The supernatants were transferred to glass tubes and 50 ug BSA (Sigma Chemical Co., St. Louis, Mo.) was added as carrier prior to adding 0.2% volume of 50% (w/v) trichloroacetic acid (Fisher Scientific, Fair Lawn, N.J.). The samples were allowed to precipitate for 24 hours at 4 degrees centigrade, transferred to 1.5 ml microfuge tubes and centrifuged for 20 minutes at 4 degrees centigrade at 3000× g. Pellets were rinsed with acetone, centrifuged immediately, supernatant removed and allowed to dry at room temperature. The pellets were resuspended in 25 ul of sample extraction buffer (SEB) (0.05 M TrisHCl, pH 7.0, 8 M urea, 1% w/v SDS and 0.01% v/v beta mercaptoethanol). The cells attached to the plates after supernatant removal were washed with cold phosphate buffered saline (PBS) and solubilized in SEB, and protein concentration determined. The samples, both from the supernatant and from the attached cells were thus ready for running in 5% SDS PAGE gels. Western blotting was performed using standard procedures. The primary antibody used was mouse monoclonal anti-human Muc1 antibody ab696 (AbCam Inc. Cambridge, Mass.), that recognizes the tandem repeat units of the MUC1 receptor, at a 1:4000 dilution. The secondary antibody was an anti-mouse HRP, Calbiochem, La Jolla, Calif.) and was used at a dilution of 1:10,000.

### Example 6

This example demonstrates that Compound No. 173 chelates Mg as evidenced by mass spectroscopy. To an aqueous solution of Mg(NO₃)₂ was added an ethanolic solution of Compound No. 173. The final concentration of metal ion and compound was 20 uM. The final concentration of ethanol was 2% (v/v). The sample was vortexed and held at room temperature. The sample was ionized using a MicroMass LCT mass spectrometer utilizing electrospray ionization. (Harvard University, Cambridge, Mass.). A signal corresponding to the 1:1 complex was observed (m/z=539). Control experiments to check for the incidental leaching of sodium from the glassware into the sample confirmed the formation of the magnesium complex. (See FIG. 23)

### Example 7

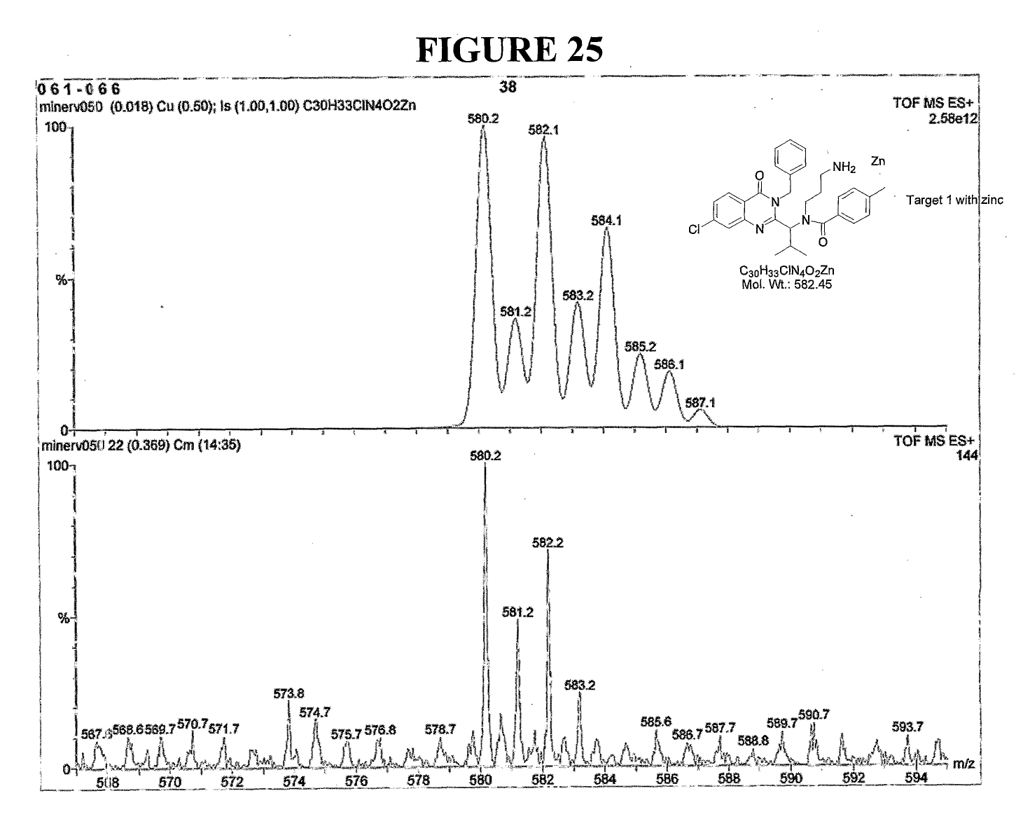

This example demonstrates that Compound No. 173 chelates Zn as evidenced by mass spectroscopy. To an aqueous solution of Zn(NO₃)₂ was added an ethanolic solution of Compound No. 173. The final concentration of metal ion and compound was 20 uM. The final concentration of ethanol was 2% (v/v). The sample was vortexed and held at room temperature. The sample was ionized using a MicroMass LCT mass spectrometer utilizing electrospray ionization. The spectrum (m/z=580) from the sample was obtained and compared to the simulated spectrum of the anticipated complex which was generated using a software package Masslynx 3.2 (Harvard University). The experimentally obtained spectrum matches the theoretically predicted spectrum for Compound No. 173 chelating Zn (See FIG. 25).

### Example 8

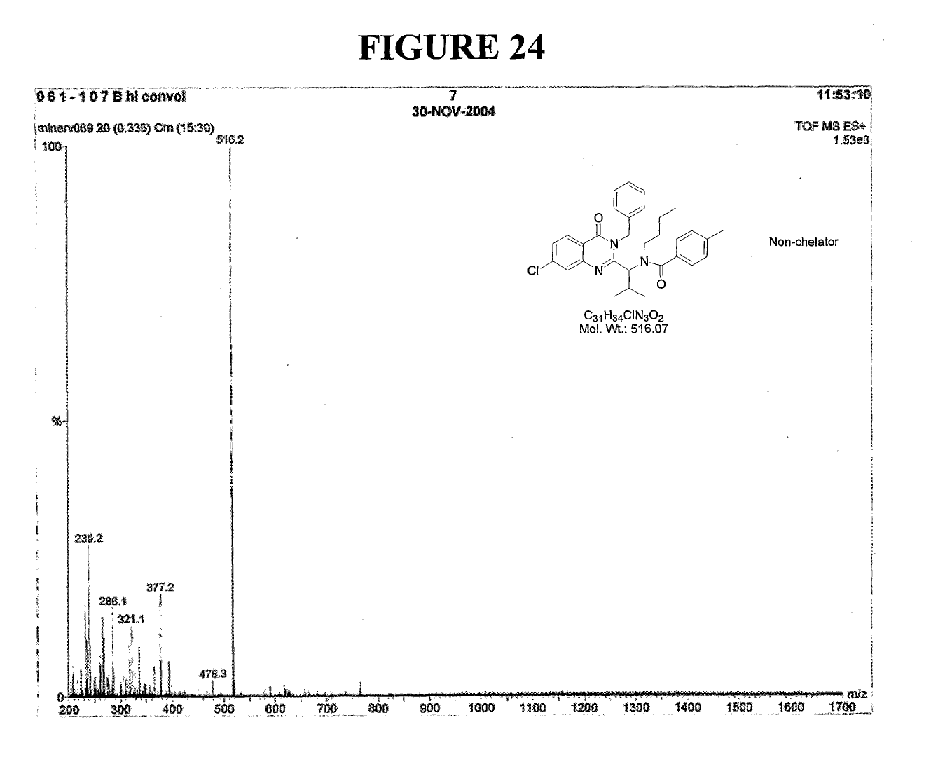

This example demonstrates that Compound No. 186, which is a derivative of No. 173, designed not to chelate metals, does not, as evidenced by mass spectroscopy. To an aqueous solution of Zn(NO₃)₂ was added an ethanolic solution of Compound 186. The final concentration of metal ion and compound was 20 uM. The final concentration of ethanol was 2% (v/v). The sample was vortexed and held at room temperature. The sample was ionized using a MicroMass LCT mass spectrometer utilizing electrospray ionization. No complex was observed (m/z=579) (See FIG. 24) under conditions identical to those used to observe the complex between Compound No. 173 and zinc (m/z=580).

### Example 9

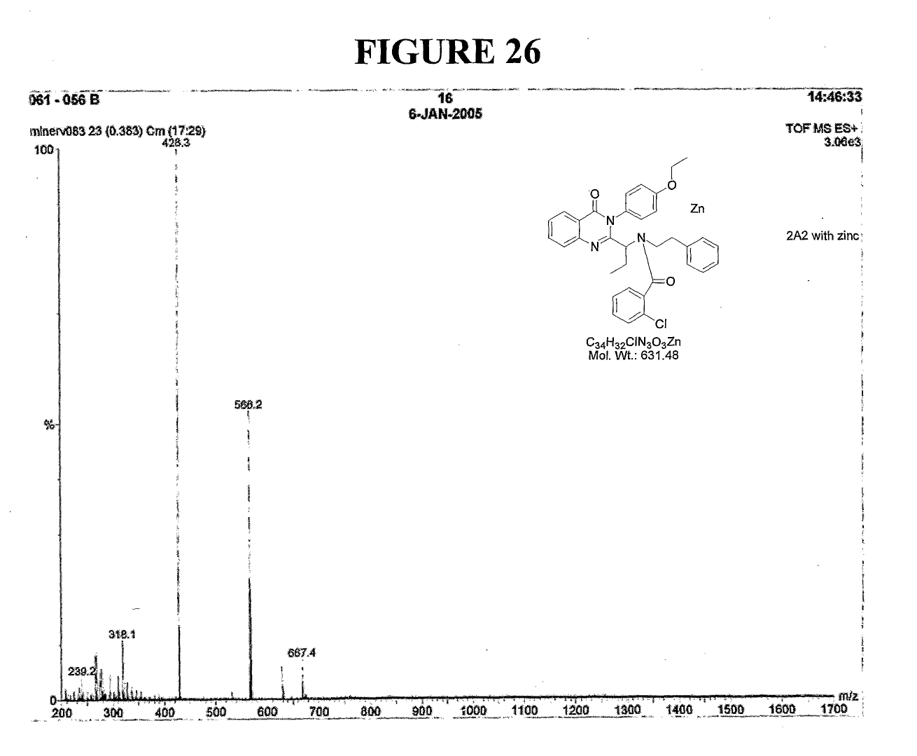

This example demonstrates that Compound No. 28 chelates Zn as evidenced by mass spectroscopy. To an aqueous solution of Zn(NO₃)₂ was added an ethanolic solution of Compound No. 28. The final concentration of metal ion and compound was 20 uM. The final concentration of ethanol was 2% (v/v). The sample was vortexed and held at room temperature. The sample was ionized using a MicroMass LCT mass spectrometer utilizing electrospray ionization. A signal corresponding to the 1:1 complex was observed (m/z=631) (See FIG. 26).

### Example 10

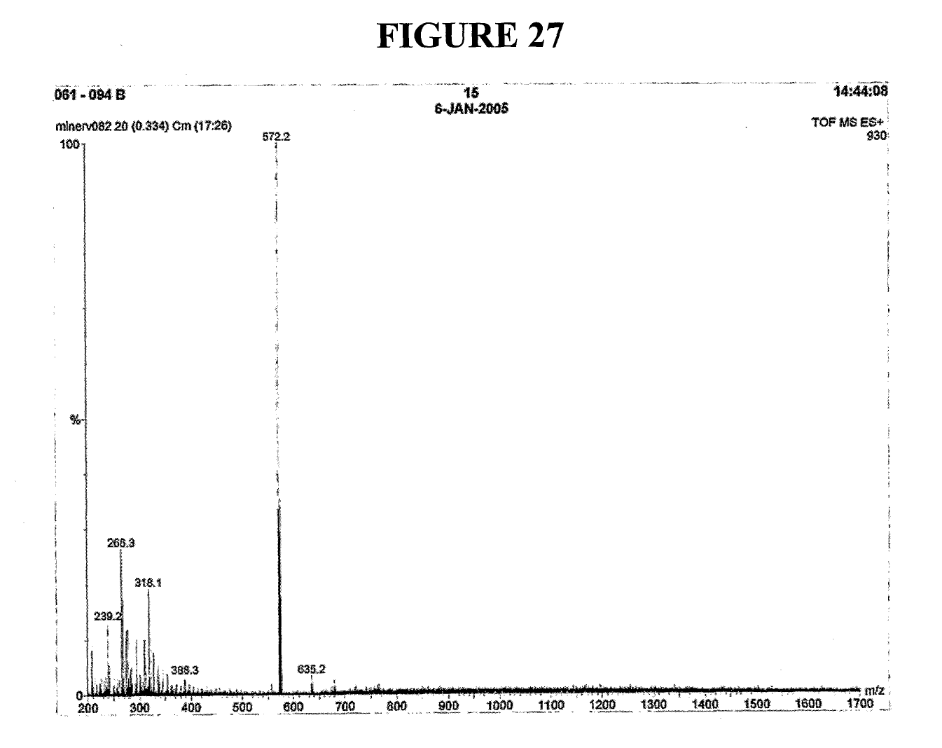

This example demonstrates that Compound No. 185 chelates Zn as evidenced by mass spectroscopy. To an aqueous solution of Zn(NO₃)₂ was added an ethanolic solution of Compound 185. The final concentration of metal ion and compound was 20 uM. The final concentration of ethanol was 2% (v/v). The sample was vortexed and held at room temperature. The sample was ionized using a MicroMass LCT mass spectrometer utilizing electrospray ionization. A signal corresponding to the 1:1 complex was observed (m/z=638) (See FIG. 27).

### Example 11

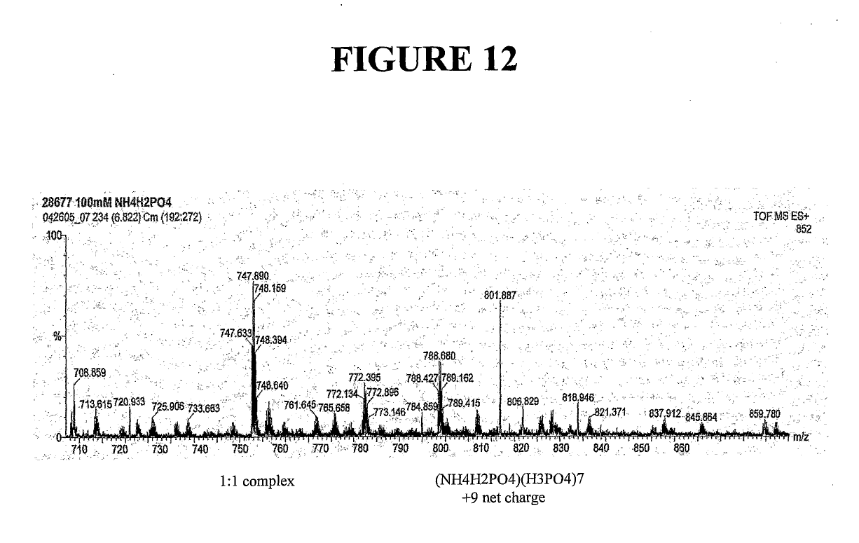

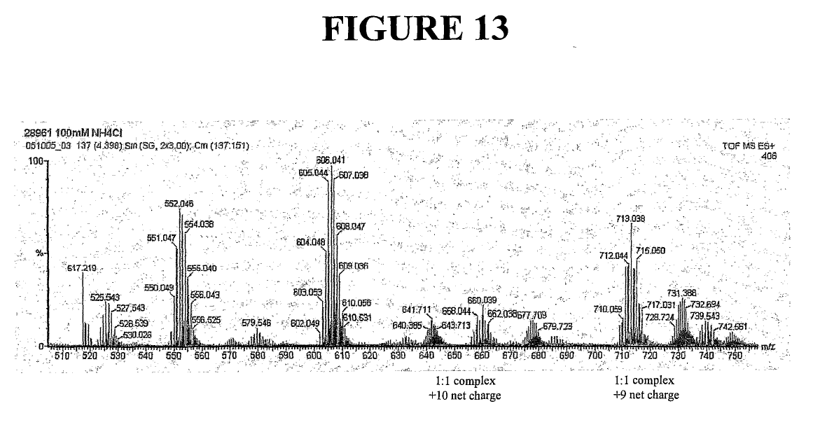

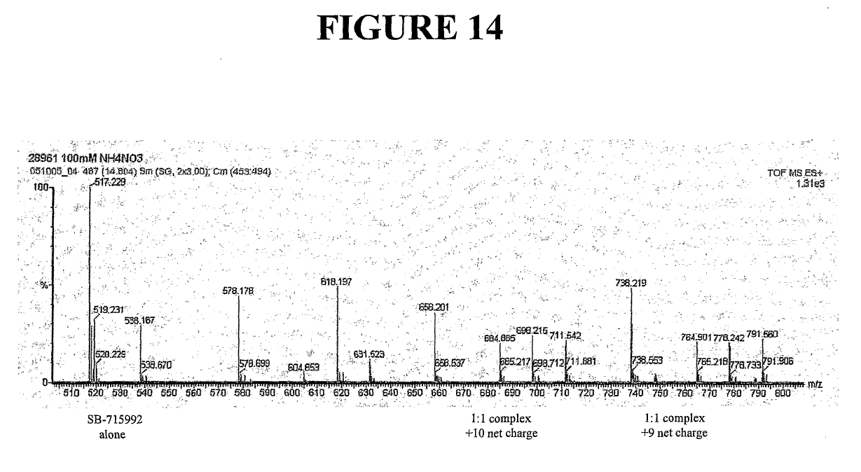

This example demonstrates that Compound No. 173 directly binds to the PSMGFR portion of the MUC1 receptor, as evidenced by mass spectroscopy. ESI⁺ MS of the PSMGFR peptide and Compound No. 173 complex. To an aqueous solution containing H₂N-GTINV HDVET QFNQY KTEAA SRYNL TISDV SVSDV PFPFS AQSGA HHhHHH-CO₂H (SEQ ID NO:29) (Quality Controlled Biochemicals, Hopkinton, Mass.) was added an equimolar amount of Compound No. 173 as the HCl salt dissolved in water. The sample was vortexed and held at ambient temperature. Immediately prior to analysis, the solution was buffered with ammonium dihydrogen phosphate. The sample was ionized with a MicroMass Q-Tof² mass spectrometer (Proteomics Research Services, Inc., Ann Arbor, Mich.) operating in the reflectron mode. A signal (m/z=748) corresponding to the 1:1 complex of the peptide with the organic compound complexed with five inorganic phosphates and an overall charge of +9 was observed. (See FIGS. 12-14).

### Example 12

This example demonstrates that Compound No. 173 directly binds to the PSMGFR portion of the MUC1 receptor, as evidenced by surface plasmon resonance. Self-assembled monolayers that incorporated a sulfur-terminated PSMGFR peptide into a background of tri-ethylene glycol terminated thiols were formed on Ti/Au-coated glass slides that were prepared for use in a Leica SPR instrument. Several SAM-coated chips were prepared that presented increasing densities of PSMGFR peptide on the surfaces. Once inserted into the SPR instrument, Compound No. 173 was introduced to the surface, then rinsed away. Results showed that as the density of PSMGFR peptide increased on the chip surface, so did the amount of Compound 173 that bound. Control peptides did not bind to the PSMGFR peptide surfaces.

### Example 13

This example describes how compounds are tested for their ability to inhibit the growth of MUC1-positive tumor cells or MUC1 transfected cells. Approximately 10,000 cells were plated into each well of a 96 well plate in 100 ul media (DMEM containing 4.5 g/L glucose, L-glutamine and sodium pyruvate, Mediatech, Inc. Herndon, Va.). The cells were allowed to attach overnight (approximately 16-20 hours). The next day, either 1 ul DMSO or 1 ul of individual compounds in DMSO were added to the wells to achieve a final concentration that ranged from 1-5 uM. Each condition was tested in at least in triplicate. Immediately prior to adding the compounds, the zero hour cell count was taken by counting the cells present in a set (3 or 5) of control wells. For counting, the media in the well was removed by aspiration and 50 ul Trypsin (Trypsin EDTA 1X, Mediatech, Inc. Herndon, Va.) was added. The cells were allowed to incubate for 5 minutes. Next, the cells were resuspended thoroughly and counted using a hemacytometer. After the compounds were added, the cells were allowed to grow in a humidified 37 degrees centigrade incubator with 5% CO₂ for 48 hours. Subsequently, the 48 hour cell count was taken in a manner identical to the zero hour cell count. Percentage cell growth was plotted as a function of compound concentration. Controls included mock drug addition, addition of irrelevant compounds. Compounds were added to MUC1-positive tumor cells and, as a control, to MUC1-negative tumor and non-tumor cells.

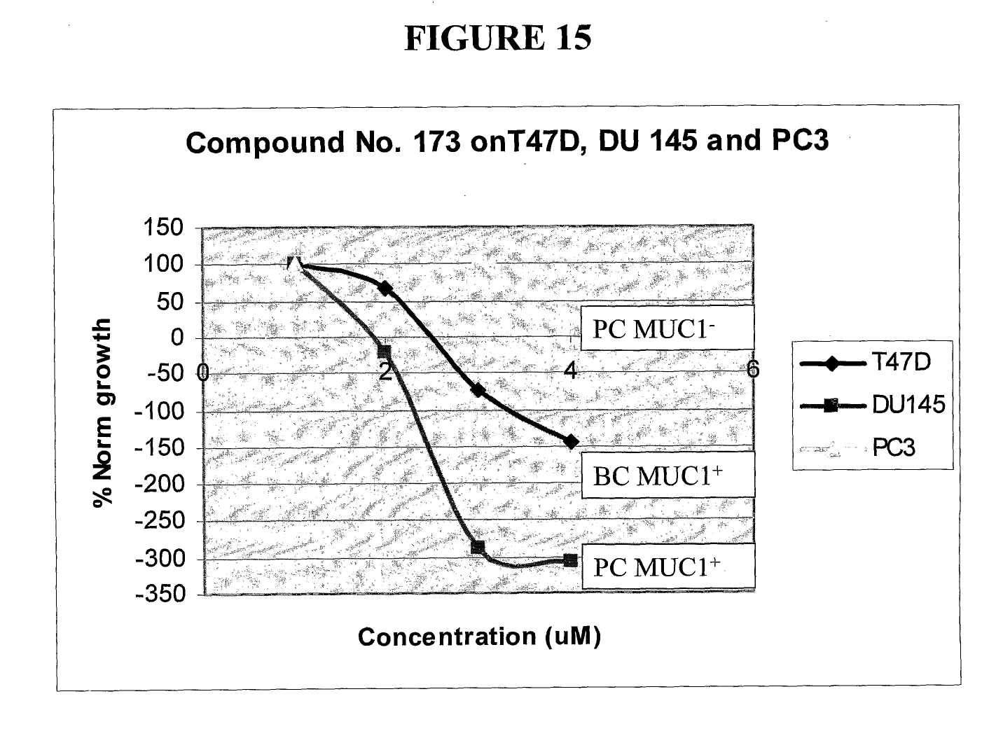

To further demonstrate that Compounds Nos. 1-188 inhibit cell growth by blocking the MUC1 receptor, the compounds were added to a panel of MUC1-negative cells, HEK 293 (human embryonic kidney), that had been transfected with DNA that caused the expression of MUC1 on the surface of the cells. MUC1 expression was confirmed by western blot, fluorescence microscopy and FACs sorting. These transfectants are described in PCT/US2004/027954, filed Aug. 26, 2004. FIG. 15 shows that Compound No. 173 preferrentially inhibits the growth of MUC1-positive tumor cells.

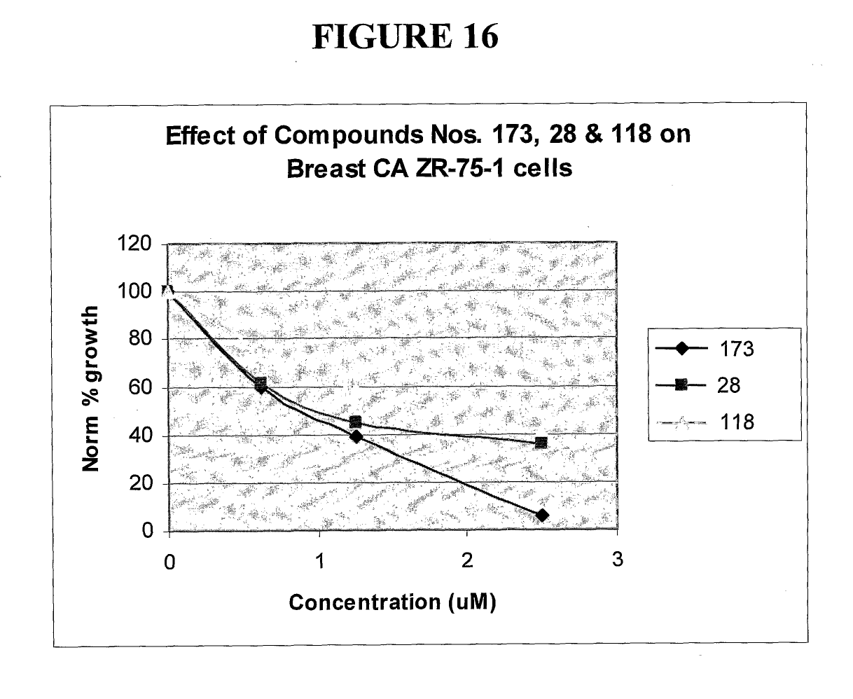

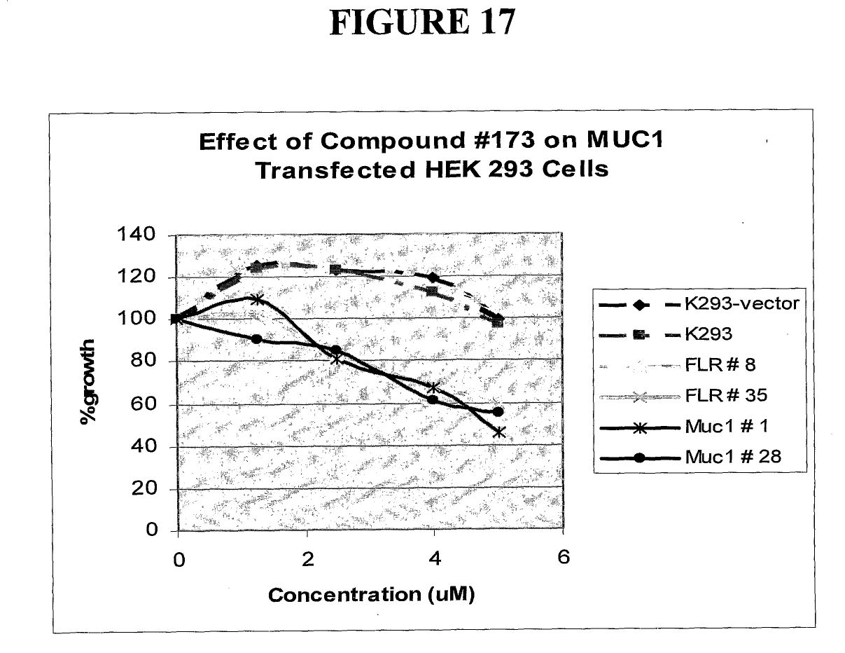

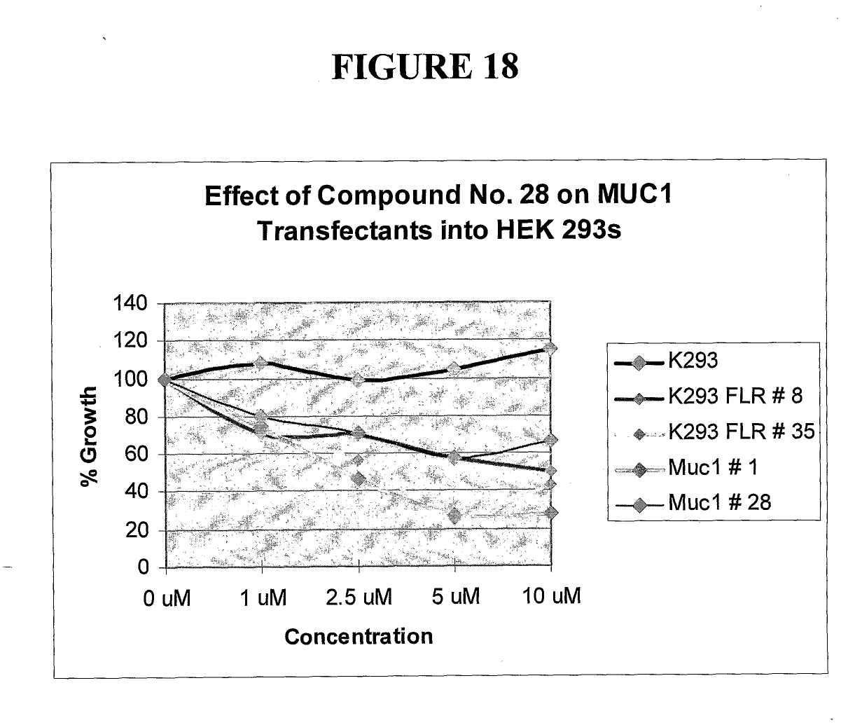

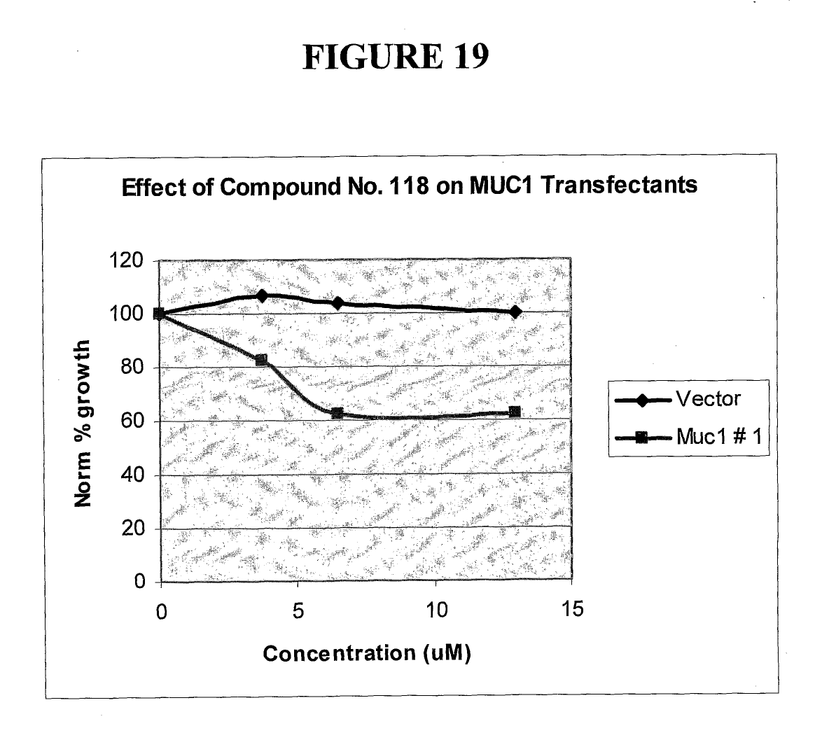

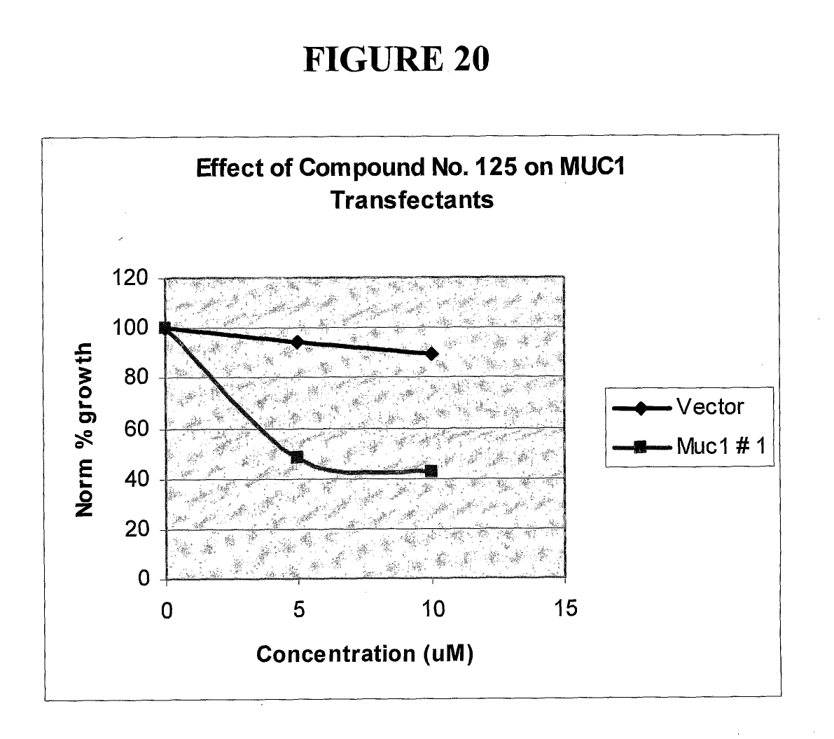

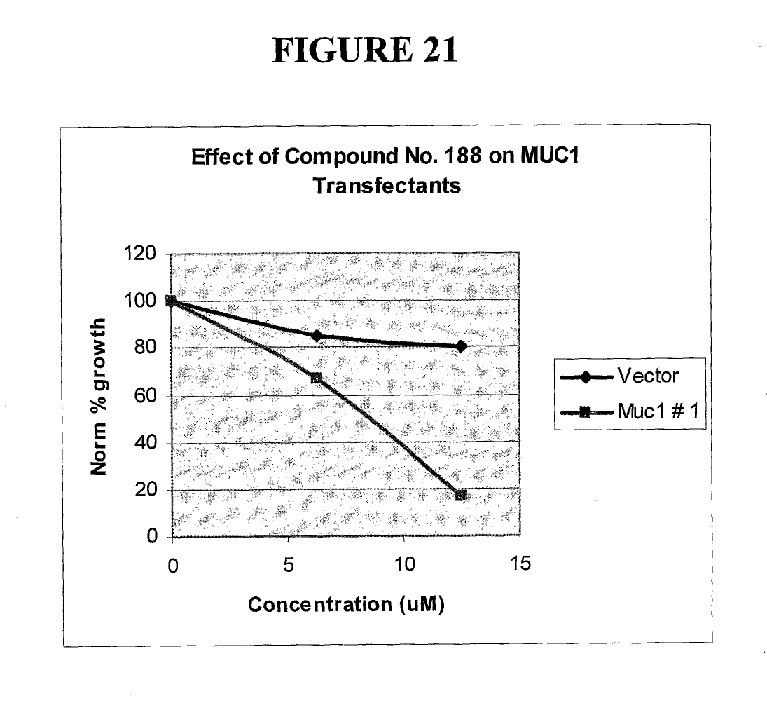

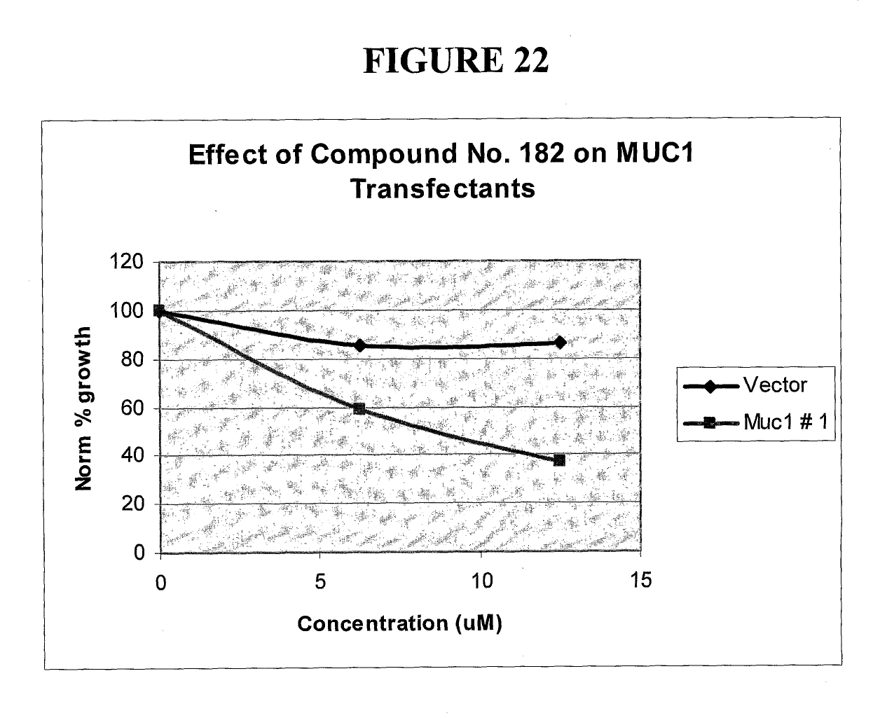

In this experiment the compound was given to three (3) tumor cell lines: PC MUC1⁻, which is PC3 a MUC1-negative prostate tumor cell line, PC MUC1⁺, DU 145 which is a MUC1-positive prostate tumor cell line and BC MUC1⁺, T47D, a MUC1-positive breast tumor cell line. As the plot of cell growth indicates, Compound No. 173 has a definitive killing effect on the MUC1-positive cells. FIG. 16 shows effects of Compounds Nos. 173, 28 and 118. All have similar inhibitory effects on the growth of MUC1-positive breast tumor cell line ZR-75-1. FIG. 17 shows that Compound No. 173 does not significantly inhibit the growth of a MUC1-negative parent cell line (HEK 293), but severely inhibits the growth of those cells after they are transfected with the MUC1 receptor. FLR #8, FLR#35, Muc #1 and MUC #28 are clones wherein the cells have been transfected to express the MUC1 receptor. FLR#8 and #35 express the PSMGFR portion alone. Western blot analysis showed that MUC clones #1 and #28 have all essentially been cleaved to yield the PSMGFR portion. FIG. 18 shows that Compound No. 28 behaves comparably to Compound No. 173 in that it preferentially inhibits the growth of MUC1-positive transfectants. FIG. 19 shows that Compound 118 inhibits the growth of MUC1 transfected cells but not the parent cells transfected with the empty vector. FIG. 20 shows that Compound No. 125 behaves in the same way against the same MUC1-positive versus negative transfected cells. FIG. 21 shows the same effect when cells are treated with Compound No. 188. FIG. 22 shows the same effect when cells are treated with Compound No. 182.

### Example 14

A patient is diagnosed with a MUC1-positive cancer and treated with Compound No. 173, 184, 28, 185, 118, 125, 182, 188, 107, 109 or combinations thereof including in combination with known chemotherapy agents.

While several embodiments of the invention have been described and illustrated herein, those of ordinary skill in the art will readily envision a variety of other means and structures for performing the functions and/or obtaining the results or advantages described herein, and each of such variations or modifications is deemed to be within the scope of the present invention. More generally, those skilled in the art would readily appreciate that all parameters, dimensions, materials, and configurations described herein are meant to be exemplary and that actual parameters, dimensions, materials, and configurations will depend upon specific applications for which the teachings of the present invention are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described. The present invention is directed to each individual feature, system, material and/or method described herein. In addition, any combination of two or more such features, systems, materials and/or methods, if such features, systems, materials and/or methods are not mutually inconsistent, is included within the scope of the present invention.

In the claims (as well as in the specification above), all transitional phrases such as “comprising,” “including,” “carrying,” “having,” “containing,” “involving,” and the like are to be understood to be open-ended, i.e. to mean including but not limited to. Only the transitional phrases “consisting of” and “consisting essentially of” shall be closed or semi-closed transitional phrases, respectively, as set forth in the United States Patent Office Manual of Patent Examining Procedures, section 2111.03.

What is claimed is:

1. A method for treating or preventing cancer in a subject comprising administering a treatment effective amount of a compound belonging to Formulae I, II, III, IV, V, VI, VII, VIII, IX, X, XI, or XII to a subject in need thereof, wherein 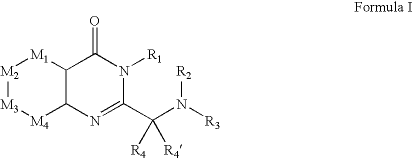
where M₁, M₂, M₃ and M₄ are each independently selected from the group consisting of carbon, nitrogen, sulfur, oxygen residues and/or an atomic null such that a fused bicyclic ring system providing valence satisfaction and chemical stability is achieved; substitutions at positions M₁-M₄ on the above atoms may be hydrogen or halogen, such as fluorine, chlorine or bromine, or carbon-linked substituents such as methyl, ethyl, propyl, isopropyl and higher alkyl and aryl analogs, or nitrogen-linked substituents including amine, methylamine, dimethylamine or higher secondary or tertiary alkyl or aryl amines, or oxygen-, sulfur- and selenium-linked substituents including hydroxyl, sulfhydryl and selenylhydryl and alkyl and aryl ether analogs thereof, or silicon-, phosphorous- or boron-linked substituents including alkyl or aryl substitutions at these residues, wherein in all cases, linking atoms and linking-atom substituents are as required for valence satisfaction and chemical stability; R₁ may be any atom or substituent other than halogen including hydrogen, methyl, ethyl, benzyl, aryl and substituted analogs thereof; R₂ and R₃ are each independently chosen to be hydrogen, or substituted oxygen-, carbon-, nitrogen- or sulfur-linked substituents such that valence satisfaction and chemical stability are achieved; R₂ and R₃ may be covalently linked to give a set of monocyclic aza-cycles; R₄ and R_{4′} may be independently hydrogen, carbon-, oxygen-, nitrogen- or sulfur-linked substituents including methyl, ethyl, isopropyl, higher alkyl and aryl substituents, hydroxyl, sulfhydryl, alkyl and aryl ethers and thioethers, amine, methyl amine, dimethyl amine and higher alkyl and aryl secondary and tertiary amines, wherein in all cases, substitutions are chosen such that valence satisfaction and chemical stability are achieved; R₄ and R_{4′} may be covalently linked to give a set of cyclic compounds; 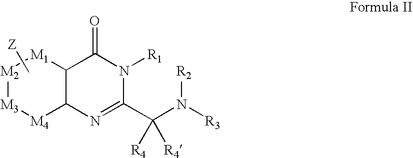
where M₁, M₂, M₃ and M₄ are each independently selected from the group consisting of carbon, nitrogen, sulfur, oxygen atoms and/or an atomic null such that a fused bicyclic ring system providing valence satisfaction and chemical stability is achieved; single and multiple substitutions by the substituent(s) Z at positions M₁-M₄ on the above designated carbon, nitrogen and sulfur atoms may be hydrogen or halogen, such as fluorine, chlorine or bromine, or carbon-linked substituents such as methyl, ethyl, propyl, isopropyl and higher alkyl and aryl analogs, or nitrogen-linked substituents including amine, methylamine, dimethylamine or higher secondary or tertiary alkyl or aryl amines, or oxygen-, sulfur- and selenium-linked substituents including hydroxyl, sulfhydryl and selenylhydryl and alkyl and aryl ether analogs thereof, or silicon-, phosphorous- or boron-linked substituents including alkyl or aryl substitutions at these atoms, wherein in all cases, linking atoms and linking-atom substituents are as required for valence satisfaction and chemical stability; R₁ may be any residue other than halogen, including hydrogen, methyl, ethyl, benzyl, aryl and substituted analogs thereof; R₂ and R₃ are each independently chosen to be hydrogen, or substituted oxygen-, carbon-, nitrogen- or sulfur-linked substituents such that valence satisfaction and chemical stability are achieved; R₂ and R₃ may be covalently linked to give a set of monocyclic aza-cycles; R₄ and R_{4′} may be independently hydrogen, carbon-, oxygen-, nitrogen- or sulfur-linked substituents including methyl, ethyl, isopropyl, higher alkyl and aryl substituents, hydroxyl, sulfhydryl, alkyl and aryl ethers and thioethers, amine, methyl amine, dimethyl amine and higher alkyl and aryl secondary and tertiary amines, wherein in all cases, substitutions are chosen such that valence satisfaction and chemical stability are achieved; R₄ and R_{4′} may be covalently linked to give a set of cyclic compounds; 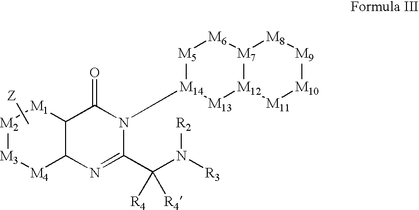
where M₁, M₂, M₃ and M₄ are each independently selected from the group consisting of carbon, nitrogen, sulfur, oxygen residue and/or an atomic null such that a fused bicyclic ring system providing valence satisfaction and chemical stability is achieved; single and multiple substitutions by the substituent(s) Z at positions M₁-M₄ on the above designated carbon, nitrogen and sulfur atoms may be hydrogen or halogen, such as fluorine, chlorine or bromine, or carbon-linked substituents such as methyl, ethyl, propyl, isopropyl and higher alkyl and aryl analogs, or nitrogen-linked substituents including amine, methylamine, dimethylamine or higher secondary or tertiary alkyl or aryl amines, or oxygen-, sulfur- and selenium-linked substituents including hydroxyl, sulfhydryl and selenylhydryl and alkyl and aryl ether analogs thereof, or silicon-, phosphorous- or boron-linked substituents including alkyl or aryl substitutions at these atoms, wherein in all cases, linking residues and linking-atom substituents are as required for valence satisfaction and chemical stability; M₅-M₁₄ may be carbon, nitrogen, oxygen or sulfur or any residue other than hydrogen or halogen and, in certain embodiments, may be either a moiety where M₅, M₆, M₇, M₈, M₉, M₁₀, M₁₁, M₁₂, M₁₃ and M₁₄ are each independently selected from the group consisting of carbon, nitrogen, sulfur, oxygen atoms and/or an atomic null such that a monocyclic or bicyclic ring system providing valence satisfaction and chemical stability are achieved; R₂ and R₃ are each independently chosen to be hydrogen, or substituted oxygen-, carbon-, nitrogen- or sulfur-linked substituents such that valence satisfaction and chemical stability are achieved; R₂ and R₃ may be covalently linked to give a set of monocyclic aza-cycles; R₄ and R_{4′} may be independently hydrogen, carbon-, oxygen-, nitrogen- or sulfur-linked substituents including methyl, ethyl, isopropyl, higher alkyl and aryl substituents, hydroxyl, sulfhydryl, alkyl and aryl ethers and thioethers, amine, methyl amine, dimethyl amine and higher alkyl and aryl secondary and tertiary amines, wherein in all cases, substitutions are chosen such that valence satisfaction and chemical stability are achieved; R₄ and R_{4′} may be covalently linked to give a set of cyclic compounds; 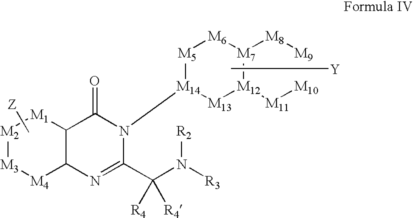
where M₁, M₂, M₃ and M₄ are each independently selected from the group consisting of carbon, nitrogen, sulfur, oxygen residue and/or an atomic null such that a fused bicyclic ring system providing valence satisfaction and chemical stability is achieved; single and multiple substitutions by the substituent(s) Z at positions M₁-M₄ on the above designated carbon, nitrogen and sulfur atoms may be hydrogen or halogen, such as fluorine, chlorine or bromine, or carbon-linked substituents such as methyl, ethyl, propyl, isopropyl and higher alkyl and aryl analogs, or nitrogen-linked substituents including amine, methylamine, dimethylamine or higher secondary or tertiary alkyl or aryl amines, or oxygen-, sulfur- and selenium-linked substituents including hydroxyl, sulfhydryl and selenylhydryl and alkyl and aryl ether analogs thereof, or silicon-, phosphorous- or boron-linked substituents including alkyl or aryl substitutions at these atoms, wherein in all cases, linking residues and linking-group substituents are as required for valence satisfaction and chemical stability; M₅-M₁₄ may be carbon, nitrogen, oxygen or sulfur or any atom other than hydrogen or halogen and, in certain embodiments, may be either a moiety where M₅, M₆, M₇, M₈, M₉, M₁₀, M₁₁, M₁₂, M₁₃ and M₁₄ are each independently selected from the group consisting of carbon, nitrogen, sulfur, oxygen atoms and/or an atomic null such that a monocyclic or bicyclic ring system providing valence satisfaction and chemical stability are achieved; furthermore, single and multiple substitutions by the substituent(s) Y at positions M₅-M₁₄ on the above designated carbon, nitrogen and sulfur atoms may be hydrogen or halogen, or substituted oxygen-, carbon-, nitrogen- or sulfur-linked substituents including methyl, ethyl, isopropyl, higher alkyl and aryl substituents, hydroxyl, sulfhydryl, alkyl and aryl ethers and thioethers, amine, methyl amine, dimethyl amine and higher alkyl and aryl secondary and tertiary amines such that valence satisfaction and chemical stability are achieved; R₂ and R₃ are each independently chosen to be hydrogen, oxygen-, carbon-, nitrogen- or sulfur-linked substituents including methyl, ethyl, isopropyl, higher alkyl and aryl substituents, hydroxyl, sulfhydryl, alkyl and aryl ethers and thioethers, amine, methyl amine, dimethyl amine and higher alkyl and aryl secondary and tertiary amines for valence satisfaction; R₂ and R₃ may be covalently linked to give a set of monocyclic aza-cycles; R₄ and R_{4′} may be independently hydrogen, carbon-, oxygen-, nitrogen- or sulfur-linked substituents including methyl, ethyl, isopropyl, higher alkyl and aryl substituents, hydroxyl, sulfhydryl, alkyl and aryl ethers and thioethers, amine, methyl amine, dimethyl amine and higher alkyl and aryl secondary and tertiary amines, wherein in all cases, substitutions are chosen such that valence satisfaction and chemical stability are achieved; R₄ and R_{4′} may be covalently linked to give a set of cyclic compounds; 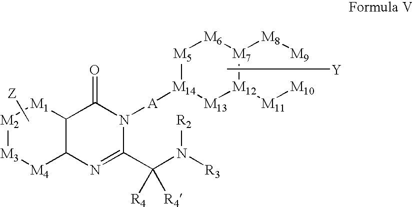
where M₁, M₂, M₃ and M₄ are each independently selected from the group consisting of carbon, nitrogen, sulfur, oxygen residue and/or an atomic null such that a fused bicyclic ring system providing valence satisfaction and chemical stability is achieved; single and multiple substitutions by the substituent(s) Z at positions M₁-M₄ on the above designated carbon, nitrogen and sulfur atoms may be hydrogen or halogen, such as fluorine, chlorine or bromine, or carbon-linked substituents such as methyl, ethyl, propyl, isopropyl and higher alkyl and aryl analogs, or nitrogen-linked substituents including amine, methylamine, dimethylamine or higher secondary or tertiary alkyl or aryl amines, or oxygen-, sulfur- and selenium-linked substituents including hydroxyl, sulfhydryl and selenylhydryl and alkyl and aryl ether analogs thereof, or silicon-, phosphorous- or boron-linked substituents including alkyl or aryl substitutions at these atoms, wherein in all cases, linking atoms and linking-atom substituents are as required for valence satisfaction and chemical stability; in certain embodiments, -A- may be any disubstituted residue, such as oxygen or sulfur, or a trisubstituted residue, such as nitrogen, or a tetrasubstituted residue, such as carbon, or any other residue capable of forming two or more stable bonds; furthermore, M₅, M₆, M₇, M₈, M₉, M₁₀, M₁₁, M₁₂, M₁₃ and M₁₄ are each independently selected from the group consisting of carbon, nitrogen, sulfur, oxygen atoms and/or an atomic null such that a monocyclic or bicyclic ring system providing valence satisfaction and chemical stability are achieved; furthermore, single and multiple substitutions by the substituent(s) Y at positions M₅-M₁₄ on the above designated carbon, nitrogen and sulfur atoms may be hydrogen or halogen, such as fluorine, chlorine or bromine, or substituted oxygen-, carbon-, nitrogen- or sulfur-linked substituents including methyl, ethyl, isopropyl, higher alkyl and aryl substituents, hydroxyl, sulfhydryl, alkyl and aryl ethers and thioethers, amine, methyl amine, dimethyl amine and higher alkyl and aryl secondary and tertiary amines such that valence satisfaction and chemical stability are achieved; R₂ and R₃ are each independently chosen to be hydrogen, oxygen-, carbon-, nitrogen- or sulfur-linked substituents including methyl, ethyl, isopropyl, higher alkyl and aryl substituents, hydroxyl, sulfhydryl, alkyl and aryl ethers and thioethers, amine, methyl amine, dimethyl amine and higher alkyl and aryl secondary and tertiary amines for valence satisfaction; R₂ and R₃ may be covalently linked to give a set of monocyclic aza-cycles; R₄ and R_{4′} may be independently hydrogen, carbon-, oxygen-, nitrogen- or sulfur-linked substituents including methyl, ethyl, isopropyl, higher alkyl and aryl substituents, hydroxyl, sulfhydryl, alkyl and aryl ethers and thioethers, amine, methyl amine, dimethyl amine and higher alkyl and aryl secondary and tertiary amines, wherein in all cases, substitutions are chosen such that valence satisfaction and chemical stability are achieved; R₄ and R_{4′} may be covalently linked to give a set of cyclic compounds; 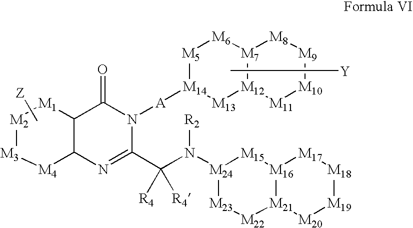
where M₁, M₂, M₃ and M₄ are each independently selected from the group consisting of carbon, nitrogen, sulfur, oxygen residue and/or an atomic null such that a fused bicyclic ring system providing valence satisfaction and chemical stability is achieved, single and multiple substitutions by the substituent(s) Z at positions M₁-M₄ on the above designated carbon, nitrogen and sulfur atoms may be hydrogen or halogen, such as fluorine, chlorine or bromine, or carbon-linked substituents such as methyl, ethyl, propyl, isopropyl and higher alkyl and aryl analogs, or nitrogen-linked substituents including amine, methylamine, dimethylamine or higher secondary or tertiary alkyl or aryl amines, or oxygen-, sulfur- and selenium-linked substituents including hydroxyl, sulfhydryl and selenylhydryl and alkyl and aryl ether analogs thereof, or silicon-, phosphorus- or boron-linked substituents including alkyl or aryl substitutions at these residues, wherein in all cases, linking residues and linking-atom substituents are as required for valence satisfaction and chemical stability; in certain embodiments, -A- may be any disubstituted residue, such as oxygen or sulfur, or a trisubstituted residue, such as nitrogen, or a tetrasubstituted residue, such as carbon, or any other atom capable of forming two or more stable bonds; furthermore, M₅, M₆, M₇, M₈, M₉, M₁₀, M₁₁, M₁₂, M₁₃ and M₁₄ are each independently selected from the group consisting of carbon, nitrogen, sulfur, oxygen atoms and/or an atomic null such that a monocyclic or bicyclic ring system providing valence satisfaction and chemical stability are achieved; single and multiple substitutions by the substituent(s) Y at positions M₅-M₁₄ on the above designated carbon, nitrogen and sulfur atoms may be hydrogen or halogen, such as fluorine, chlorine or bromine, or substituted oxygen-, carbon-, nitrogen- or sulfur-linked substituents including methyl, ethyl, isopropyl, higher alkyl and aryl substituents, hydroxyl, sulfhydryl, alkyl and aryl ethers and thioethers, amine, methyl amine, dimethyl amine and higher alkyl and aryl secondary and tertiary amines such that valence satisfaction and chemical stability are achieved; R₂ and R₃ are each independently chosen to be hydrogen, oxygen-, carbon-, nitrogen- or sulfur-linked substituents including methyl, ethyl, isopropyl, higher alkyl and aryl substituents, hydroxyl, sulfhydryl, alkyl and aryl ethers and thioethers, amine, methyl amine, dimethyl amine and higher alkyl and aryl secondary and tertiary amines for valence satisfaction; R₂ and R₃ may be covalently linked to give a set of monocyclic, aza-cycles; wherein in certain embodiments, R₂ is a moiety containing two residues other than hydrogen and no more than eight residues other than hydrogen; M₁₅-M₂₄ may be independently carbon, nitrogen, oxygen or sulfur or any residue other than hydrogen or halogen and, in certain embodiments, may be either a moiety where M₁₅, M₁₆, M₁₇, M₁₈, M₁₉, M₂₀, M₂₁, M₂₂, M₂₃ and M₂₄ are each independently selected from the group consisting of carbon, nitrogen, sulfur, oxygen residue and/or an atomic null such that a monocyclic or bicyclic ring system providing valence satisfaction and chemical stability are achieved; R₄ and R_{4′} may be independently hydrogen, carbon, oxygen, nitrogen or sulfur with substitutions as needed for valence satisfaction; R₄ and R_{4′} may be covalently linked to give a set of cyclic compounds; 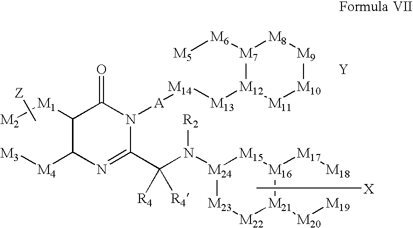
where M₁, M₂, M₃ and M₄ are each independently selected from the group consisting of carbon, nitrogen, sulfur, oxygen atoms and/or an atomic null such that a fused bicyclic ring system providing valence satisfaction and chemical stability is achieved; single and multiple substitutions by the substituent(s) Z at positions M₁-M₄ on the above designated carbon, nitrogen and sulfur residues may be hydrogen or halogen, such as fluorine, chlorine or bromine, or carbon-linked substituents such as methyl, ethyl, propyl, isopropyl and higher alkyl and aryl analogs, or nitrogen-linked substituents including amine, methylamine, dimethylamine or higher secondary or tertiary alkyl or aryl amines, or oxygen-, sulfur- and selenium-linked substituents including hydroxyl, sulflhydryl and selenylhydryl and alkyl and aryl ether analogs thereof, or silicon-, phosphorus- or boron-linked substituents including alkyl or aryl substitutions at these atoms, wherein in all cases, linking residues and linking-atom substituents are as required for valence satisfaction and chemical stability, wherein in certain embodiments, -A- may be any disubstituted residue, such as oxygen or sulfur, or a trisubstituted residue, such as nitrogen, or a tetrasubstituted residue, such as carbon, or any other residuecapable of forming two or more stable bonds; furthermore, M₅, M₆, M₇, M₈, M₉, M₁₀, M₁₁, M₁₂, M₁₃ and M₁₄ are each independently selected from the group consisting of carbon, nitrogen, sulfur, oxygen residue and/or an atomic null such that a monocyclic or bicyclic ring system providing valence satisfaction and chemical stability are achieved; single and multiple substitutions by the substituent(s) Y at positions M₅-M₁₄ on the above designated carbon, nitrogen and sulfur residue may be hydrogen or halogen, such as fluorine, chlorine or bromine, or substituted oxygen-, carbon-, nitrogen- or sulfur-linked substituents including methyl, ethyl, isopropyl, higher alkyl and aryl substituents, hydroxyl, sulfhydryl, alkyl and aryl ethers and thioethers, amine, methyl amine, dimethyl amine and higher alkyl and aryl secondary and tertiary amines such that valence satisfaction and chemical stability are achieved; R₂ and R₃ are each independently chosen to be hydrogen, oxygen-, carbon-, nitrogen- or sulfur-linked substituents including methyl, ethyl, isopropyl, higher alkyl and aryl substituents, hydroxyl, sulfhydryl, alkyl and aryl ethers and thioethers, amine, methyl amine, dimethyl amine and higher alkyl and aryl secondary and tertiary amines for valence satisfaction; R₂ and R₃ may be covalently linked to give a set of monocyclic aza-cycles, wherein in certain embodiments, R₂ is a moiety containing two residues other than hydrogen and no more than eight residues other than hydrogen; M₁₅-M₂₄ may be independently carbon, nitrogen, oxygen or sulfur or any residue other than hydrogen or halogen and, in certain embodiments, may be either a moiety where M₁₅, M₁₆, M₁₇, M₁₈, M₁₉, M₂₀, M₂₁, M₂₂, M₂₃ and M₂₄ are each independently selected from the group consisting of carbon, nitrogen, sulfur, oxygen residues and/or an atomic null such that a monocyclic or bicyclic ring system providing valence satisfaction and chemical stability are achieved; single and multiple substitutions by substituent(s) X at positions M₁₅-M₂₄ on the above designated carbon, nitrogen and sulfur atoms may be hydrogen or halogen, such as fluorine, chlorine or bromine, or substituted oxygen-, carbon-, nitrogen- or sulfur-linked substituents including methyl, ethyl, isopropyl, higher alkyl and aryl substituents, hydroxyl, sulfhydryl, alkyl and aryl ethers and thioethers, amine, methyl amine, dimethyl amine and higher alkyl and aryl secondary and tertiary amines such that valence satisfaction and chemical stability are achieved; R₂ and R₃ are each independently chosen to be hydrogen, oxygen-, carbon-, nitrogen- or sulfur-linked substituents including methyl, ethyl, isopropyl, higher alkyl and aryl substituents, hydroxyl, sulfhydryl, alkyl and aryl ethers and thioethers, amine, methyl amine, dimethyl amine and higher alkyl and aryl secondary and tertiary amines for valence satisfaction; R₂ and R₃ may be covalently linked to give a set of monocyclic aza-cycles; R₄ and R_{4′} may be independently hydrogen, carbon-, oxygen-, nitrogen- or sulfur-linked substituents including methyl, ethyl, isopropyl, higher alkyl and aryl substituents, hydroxyl, sulfhydryl, alkyl and aryl ethers and thioethers, amine, methyl amine, dimethyl amine and higher alkyl and aryl secondary and tertiary amines, wherein in all cases, substitutions are chosen such that valence satisfaction and chemical stability are achieved; R₄ and R_{4′} may be covalently linked to give a set of cyclic compounds; 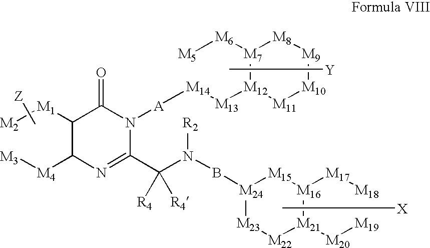
where M₁, M₂, M₃ and M₄ are each independently selected from the group consisting of carbon, nitrogen, sulfur, oxygen residue and/or an atomic null such that a fused bicyclic ring system providing valence satisfaction and chemical stability is achieved; single and multiple substitutions by the substituent(s) Z at positions M₁-M₄ on the above designated carbon, nitrogen and sulfur residues may be hydrogen or halogen, such as fluorine, chlorine or bromine, or carbon-linked substituents such as methyl, ethyl, propyl, isopropyl and higher alkyl and aryl analogs, or nitrogen-linked substituents including amine, methylamine, dimethylamine or higher secondary or tertiary alkyl or aryl amines, or oxygen-, sulfur- and selenium-linked substituents including hydroxyl, sulfhydryl and selenylhydryl and alkyl and aryl ether analogs thereof, or silicon-, phosphorus- or boron-linked substituents including alkyl or aryl substitutions at these atoms, wherein in all cases, linking atoms and linking-atom substituents are as required for valence satisfaction and chemical stability; wherein in certain embodiments, -A- may be any disubstituted residue, such as oxygen or sulfur, or a trisubstituted residue, such as nitrogen, or a tetrasubstituted residue, such as carbon, or any other residue capable of forming two or more stable bonds; furthermore, M₅, M₆, M₇, M₈, M₉, M₁₀, M₁₁, M₁₂, M₁₃ and M₁₄ are each independently selected from the group consisting of a carbon, nitrogen, sulfur, oxygen residue and/or an atomic null such that a monocyclic or bicyclic ring system providing valence satisfaction and chemical stability are achieved; single and multiple substitutions by the substituent(s) Y at positions M₅-M₁₄ on the above designated carbon, nitrogen and sulfur atoms may be hydrogen or halogen, such as fluorine, chlorine or bromine, or substituted oxygen-, carbon-, nitrogen- or sulfur-linked substituents including methyl, ethyl, isopropyl, higher alkyl and aryl substituents, hydroxyl, sulfhydryl, and aryl secondary and tertiary amines such that valence satisfaction and chemical stability are achieved; R₂ and R₃ are each independently chosen to be hydrogen, oxygen-, carbon-, nitrogen- or sulfur-linked substituents including methyl, ethyl, isopropyl, higher alkyl and aryl substituents, hydroxyl, sulfhydryl, alkyl and aryl ethers and thioethers, amine, methyl amine, dimethyl amine and higher alkyl and aryl secondary and tertiary amines for valence satisfaction; R₂ and R₃ may be covalently linked to give a set of monocyclic aza-cycles; R₂ may be a moiety containing two residues other than hydrogen and no more than eight residues other than hydrogen, wherein in certain embodiments, -B- may be any disubstituted residue, such as oxygen or sulfur, or a trisubstituted residue, such as nitrogen, or a tetrasubstituted residue, such as carbon, or any other residue capable of forming two or more stable bonds; linker -B- may be chosen as an atomic null through an octa-atomic set of non-hydrogen atoms such that valence satisfaction and chemical stability are achieved; furthermore, M₁₅-M₂₄ may be independently carbon, nitrogen, oxygen or sulfur or any residue other than hydrogen or halogen and, in certain embodiments, may be either a moiety where M₁₅, M₁₆, M₁₇, M₁₈, M₁₉, M₂₀, M₂₁, M₂₂, M₂₃ and M₂₄ are each independently selected from the group consisting of a carbon, nitrogen, sulfur, oxygen residue and/or an atomic null such that a monocyclic or bicyclic ring system providing valence satisfaction and chemical stability are achieved; single and multiple substitutions by substituent(s) X at positions M₁₅-M₂₄ on the above designated carbon, nitrogen and sulfur residue may be hydrogen or halogen, such as fluorine, chlorine or bromine, or substituted oxygen-, carbon-, nitrogen- or sulfur-linked substituents including methyl, ethyl, isopropyl, higher alkyl and aryl substituents, hydroxyl, sulfhydryl, alkyl and aryl ethers and thioethers, amine, methyl amine, dimethyl amine and higher alkyl and aryl secondary and tertiary amines such that valence satisfaction and chemical stability are achieved, wherein in all cases, substitutions are chosen such that valence satisfaction and chemical stability are achieved; R₄ and R_{4′} may be covalently linked to give a set of cyclic compounds; 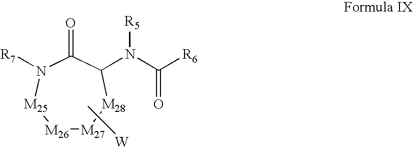
where M₂₅-M₂₈ are independently selected from the group consisting of carbon, nitrogen-sulfur, oxygen residue and/or an atomic null such that a mono-cyclic system of at least 4 residues and no more than 7 residues displaying valence satisfaction and chemical stability is achieved; substitution(s) (W) at positions M₂₅-M₂₈ on the above residues may be hydrogen or halogen, such as fluorine, chlorine or bromine, or carbon-linked substituents such as methyl, ethyl, propyl, isopropyl and higher alkyl and aryl analogs, or nitrogen-linked substituents including amine, methylamine, dimethylamine or higher secondary or tertiary alkyl or aryl amines, or oxygen-, sulfur- and selenium-linked substituents including hydroxyl, sulfhydryl and selenylhydryl and alkyl and aryl ether analogs thereof, or silicon-, phosphorous- or boron-linked substituents including alkyl or aryl substitutions at these residues, wherein in all cases, linking atoms and linking-atom substituents are as required for valence satisfaction and chemical stability; R₅ may be any carbon-linked moiety including methyl, ethyl, benzyl, aryl and substituted analogs thereof; R₆ is independently chosen to be a carbon- or nitrogen-linked substituent such that valence satisfaction and chemical stability are achieved; R₇ is hydrogen or a carbon-linked substituent such as methyl, ethyl, isopropyl, higher alkyl, benzyl or aryl and substituted analogs thereof; 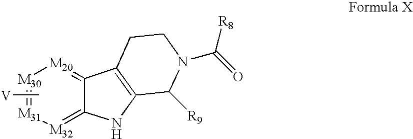
where M₂₉-M₃₂ are independently selected from the group consisting of carbon, nitrogen, sulfur, oxygen residue and/or an atomic null such that a tri-cyclic system displaying valence satisfaction and chemical stability is achieved; substitution(s) (V) at positions M₂₉-M₃₂ on the above atoms may be hydrogen or halogen, such as fluorine, chlorine or bromine, or carbon-linked substituents such as methyl, ethyl, propyl, isopropyl and higher alkyl and aryl analogs, or nitrogen-linked substituents including amine, methylamine, dimethylamine or higher secondary or tertiary alkyl or aryl amines, or oxygen-, sulfur- and selenium-linked substituents including hydroxyl, sulfhydryl and selenylhydryl and alkyl and aryl ether analogs thereof, or silicon-, phosphorous- or boron-linked substituents including alkyl or aryl substitutions at these atoms, wherein in all cases, linking atoms and linking-atom substituents are as required for valence satisfaction and chemical stability; R₈ may be any carbon- or nitrogen-linked moiety including methyl, ethyl, benzyl, aryl and substituted analogs thereof; R₉ is independently chosen to be a carbon-linked substituent such as methyl, ethyl, isopropyl, higher alkyl, benzyl or aryl and substituted analogs thereof; R₈ and R₉ may be covalently joined to give a cyclic structure; 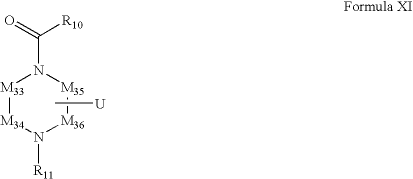
where M₃₃-M₃₆ are selected from the group consisting of carbon or nitrogen, and/or atomic null(s) such that a monocyclic or acyclic system displaying valence satisfaction and chemical stability is achieved; substitution(s) (U) at positions M₃₃-M₃₆ on the above residues may be hydrogen or carbon-linked substituents such as methyl, ethyl, propyl, isopropyl and higher alkyl and aryl analogs, or nitrogen-linked substituents including amine, methylamine, dimethylamine or higher secondary or tertiary alkyl or aryl amines, or oxygen-, sulfur- and selenium-linked substituents including hydroxyl, sulfhydryl and selenylhydryl and alkyl and aryl ether analogs thereof, or silicon-, phosphorus- or boron-linked substituents including alkyl or aryl substitutions at these atoms, wherein in all cases, linking atoms and linking-atom substituents are as required for valence satisfaction and chemical stability; R₁₀ may be any carbon- or nitrogen-linked moiety including methyl, ethyl, benzyl, aryl and substituted analogs thereof; R₁₁ is independently chosen to be a carbon-linked substituent such as methyl, ethyl, ethyl amine, isopropyl, higher alkyl, benzyl or aryl and substituted analogs thereof; 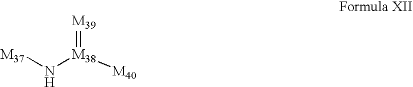
where M₃₇ is selected from the group consisting of substituted carbon, M₃₈ is selected from the group consisting of carbon and sulfur and M₃₉ is selected from either oxygen or sulfur and M₄₀ is selected from either substituted carbon or substituted nitrogen to provide a system displaying valence satisfaction and chemical stability; M₃₇ and M₄₀ may be covalently joined to provide a cyclic system; substitution(s) at positions M₃₇ and M₄₀ on the above residues may be hydrogen or carbon-linked substituents such as methyl, ethyl, propyl, isopropyl and higher alkyl and aryl analogs, or nitrogen-linked substituents including amine, methylamine, dimethylamine or higher secondary or tertiary alkyl or aryl amines, or oxygen-, sulfur- and selenium-linked substituents including hydroxyl, sulfhydryl and selenylhydryl- and alkyl and aryl ether analogs thereof, or silicon-, phosphorus- or boron-linked substituents including alkyl or aryl substitutions at these residues, wherein in all cases, linking atoms and linking-atom substituents are as required for valence satisfaction and chemical stability.

2. The method according to claim 1, wherein the compound is any one of Compound No. 1 through 188 as set forth in Tables 2 to 5.

3. The method according to claim 2, wherein the compound belonging to Formulae I, II, III, IV, V, VI, VII, or VIII is Compound No. 1-5, 7, 11, 13-15, 17-24, 26, 28-31, 42-48, 51, 55-106, 173, 174-179, 184, 185, or 186.

4. The method according to claim 3, wherein the compound is Compound No. 28, 173, 184, or 185.

5. The method according to claim 2, wherein the compound belonging to Formula IX is Compound No. 33, 50, 166-172, 180-183, or 188.

6. The method according to claim 5, wherein the compound is Compound No. 182 or 188.

7. The method according to claim 2, wherein the compound belonging to Formula X is Compound No. 8, 25, 115, 118, 120, 122-128, 130-132, or 134.

8. The method according to claim 7, wherein the compound is Compound No. 118 or 125.

9. The method according to claim 2, wherein the compound belonging to Formula XI is Compound No. 35, 107-113, or 114.

10. The method according to claim 9, wherein the compound is Compound No. 107 or 109.

11. The method according to claim 2, wherein the compound belonging to Formula XII is Compound No. 6, 9, 10, 12, 16, 27, 32, 34, 36-41, 49, 52-54, 116, 117, 119, 121, 129, 133, 135-165, or 187.

12. The method according to claim 1, comprising (i) testing a bodily sample from the subject for aberrant expression of MUC1; and (ii) treating the subject in need of the compound with the compound.

13. The method according to claim 12, wherein the compound belongs to Formulae I, II, III, IV, V, VI, VII, or VIII.

14. The method according to claim 12, wherein the compound belongs to Formulae IX, X, XI, or XII.

15. The method according to claim 1, wherein aberrant expression is loss of clustering pattern of MUC1 on apical border of cell and uniform distribution over cell surface and/or over-expression of MUC1.

16. The method according to claim 12, wherein aberrant expression is membrane staining that uniformly covers a cell when contacted with an agent or antibody that recognizes nat-PSMGFR, var-PSMGFR or ESPSMGFR peptide.

17. A method for treating or preventing MUC1-positive cancers comprising:
(i) testing a bodily sample for aberrant expression of MUC1; and
(ii) treating the patient with a compound comprising a MGFR binding region and metal chelator group wherein the metal is zinc, magnesium or nickel.

18. The method according to claim 17, wherein the compound inhibits a metal-dependent protein.

19. The method according to claim 18, wherein the protein is a kinesin.

20. The method according to claim 17, wherein the metal-dependent protein is an enzyme that cleaves MUC1.

21. The method according to claim 20, wherein the enzyme is matrix metalloprotease.

22. The method according to claim 21, wherein the enzyme is MT1-MMP or MMP-14.

23. A method for diagnosing cancer cells, comprising: (i) contacting a population of cells with a MGFR specific ligand bound to a signal generating label; and (ii) assaying for binding of the ligand to MGFR on the surface of the cells, in which the presence of uniform distribution of the signal over the entire cell surface when contacted with the ligand indicates that the cells are cancerous.
